(19) United States
(12) Patent Application Publication
(10) Pub. No.: US 2026/0217777 A1
(43) Pub. Date: Jul. 30, 2026

(54) ENGINEERED RNASE INHIBITOR VARIANTS

(71) Applicant: Codexis, Inc., Redwood City, CA (US)

(72) Inventors: Anders Matthew Knight, Mountain View, CA (US); Simon Ng, San Francisco, CA (US)

(21) Appl. No.: 19/142,170

(22) PCT Filed: Dec. 21, 2023
(86) PCT No.: PCT/US2023/085570

§ 371 Date: Jun. 20, 2025

Related U.S. Application Data
(60) Provisional application No. 63/476,599, filed on Dec. 21, 2022.

(51) Int. Cl. C07K 14/47; C12Q 1/6806

(52) U.S. Cl. CPC C07K 14/4703; C12Q 1/6806

(57) ABSTRACT

The present disclosure relates to engineered RNase inhibitor polypeptides and compositions thereof, as well as polynucleotides encoding the engineered RNase inhibitor polypeptides. The present disclosure also provides methods of using the engineered RNase inhibitor polypeptides or compositions thereof for molecular biological, diagnostic and other purposes.

### CROSS-REFERENCE TO RELATED APPLICATIONS

This application claims the benefit of U.S. Provisional Application No. 63/476,599, filed Dec. 21, 2022, which is incorporated by reference herein.

### REFERENCE TO SEQUENCE LISTING, TABLE OR COMPUTER PROGRAM

The Sequence Listing concurrently submitted herewith as file name CX9-239WO2_ST26.xml, created on Dec. 21, 2023, with a file size of 1,805,219 bytes, is part of the specification and is incorporated by reference herein.

### TECHNICAL FIELD

The present disclosure provides engineered RNase inhibitor polypeptides and compositions thereof, as well as polynucleotides encoding the engineered RNase inhibitor polypeptides. The disclosure also provides methods of using the recombinant RNase inhibitor or compositions thereof, including for RNA isolation, in vitro transcription, cDNA synthesis, RT-PCR, and other molecular biological and diagnostic purposes.

### BACKGROUND

RNase inhibitors (RI) are expressed in a variety of vertebrate cells and inhibit activity of ribonucleases (RNase) (see, e.g., Lee et al., Biochemistry, 1989, 28 (1): 225-230; Lomax et al. J Mol Biol., 2014, 426 (17): 3041-3056). RNase inhibitors may have biological roles in regulating biological activity of ribonucleases, such as in response to cellular stress, blood vessel growth, neuronal survival, and toxic response to pathogens (see, e.g., Ohashi et al., PLOS One, 2017, 12 (3): e0174237). Inhibition of RNases by RIs is attributed to the non-covalent, high affinity binding of the RI to RNases. The measured K_{d} for RI: RNase complex is in the range of 10⁻¹⁴ M to 10⁻¹⁶ M (see, e.g., Dickson et a., Prog Nucleic Acid Res Mol Biol., 2005, 80:349-374).

RNase inhibitors have practical applications in molecular and diagnostic techniques involving manipulation of RNA, such as in vitro transcription reactions, cDNA synthesis using reverse transcriptases, RT-PCR reactions, RNA purification/isolation, particularly where RNA integrity is critical and RNase contamination is a concern.

Because RNase are ubiquitous and generally stable to heat and detergents, it may contaminate reagents and laboratory instruments, such as pipets, flasks, and robotic liquid handlers. RNase may also be present in samples used as sources of the RNA, such as cellular extracts, tissues samples, and other biological samples. The presence of RNase can cause degradation of RNA compromising its integrity.

RNase inhibitors have been isolated from various sources, many of them being commercially available (see, e.g., Burton et al., Int J Pept Protein Res., 1982, 19 (4): 372-9). A prototypical RNase inhibitor is human placental RNase inhibitor, which is expressed as a single-chain polypeptide of 460 amino acid residues and contains leucine-rich repeats, a motif typically associated with protein: protein interactions. RNasin® is a recombinant form of placental RNase inhibitor produced in E. coli. RNase inhibitors from mouse and pig are also commercially available. RNase inhibitors generally do not inhibit other nucleases, reverse transcriptases or polymerases, making it a useful reagent in protecting RNA from degradation in methods employing such enzymes, such as in vitro transcription and RT-PCR. However, RNase inhibitors have some drawbacks, including low temperature stability, pH sensitivity, and sensitivity to oxidation (see, e.g., Kim et al., Protein Sci., 1999, 8 (2): 430-434). In applications involving high temperatures, such as RT-PCR, thermal inactivation of RNase inhibitors can result in release of a bound RNase, which can renature to its active form and degrade the RNA template in the sample.

### SUMMARY

The present disclosure provides engineered RNase inhibitor polypeptides and compositions thereof, as well as polynucleotides encoding the engineered RNase inhibitor polypeptides. The present disclosure also provides methods of using the engineered RNase inhibitor polypeptides and compositions thereof for nucleic acid synthesis, diagnostic assays, and other purposes.

In one aspect, the present disclosure provides an engineered RNase inhibitor, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of an even numbered SEQ ID NO. of SEQ ID NOs: 2 and 26-824, or to a reference sequence corresponding to an even numbered SEQ ID NO. of SEQ ID NOs: 2 and 26-824, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of SEQ ID NO: 2, 36, 238, 320, 360, 442, or 578, or to a reference sequence corresponding to SEQ ID NO: 2, 36, 238, 320, 360, 442, or 578, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 2, or to the reference sequence corresponding to SEQ ID NO: 2, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-142, or to a reference sequence corresponding to an even numbered SEQ ID NO. of SEQ ID NOs: 26-142, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 95, 296, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set at amino acid position(s) 257/459, 345, 257, 296, 113, 95/296/459, 95/257/296, 453, 107/267, 417/422/459, 395, 390, 257/296, 243, 156/257, 351, 348, 363, 95/257/296/422, 94/121/332/390/466, 257/417/419/459, 452, 377/390, 272, 126/386, 267, 430, 230/429, 459, 83/107/230/267/429, 107/429, 107/323/429, 94/121/265/390, 400, 429, or 81, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set of a variant provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set of an RNase inhibitor variant provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or an amino acid residue 12K, 13T, 15R, 28A/H, 35R, 37A/C, 38A/C/R/T, 40I, 50Q/S/M, 51H, 56A, 57M/T/V, 60K/S, 64R/V, 661, 78K/T, 81F/M/T/V, 83L, 84I/V, 90G, 91K, 94L, 95E/Q, 107A/D/I/V, 113G, 118R, 121S, 124A, 126V, 135V, 137V, 138L, 142H/Q, 145E/G/I/M/S/V, 147E/P, 148Q/N, 150Q, 156M, 158C, 167K, 168F/I, 173I, 175F, 176S, 177T/V, 178D, 203E, 2051/L/P/V, 221S, 228A/L/K/R/Q, 230L, 237R, 243C/M/R/S, 257F/G/S/T/V, 260L/S/Y, 265L, 267H/K/R/T, 272N/Q, 285K/Q, 291E/P/S, 296I/L, 317S, 319G/T, 323E, 326L, 332L, 338V, 341C, 342A/H/M, 345A/S, 348T/K, 351K/R, 363E/M, 367A/L/V, 373A, 377S/T, 385A, 386G, 390G/S/T, 395C/E/I/L/M/V, 396A, 400L, 417L, 419D, 422V, 429L, 430G/L, 452K/R, 453A/F/K/L/M/R/W, 459E/K/M, or 466I, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, or to the reference sequence corresponding to SEQ ID NO: 36, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 144-316, or to the reference sequence corresponding to an even-numbered SEQ ID NO. of SEQ ID NOs: 144-316, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the amino acid sequence of the engineered RNase inhibitor comprises at least a substitution or substitution set at amino acid position(s) 323/390/429, 257/323/429, 257/390, 257/323/390, 257/323/390/429, 257/429, 257/377/390, 135/257/323/377/390, 323/377/390/429, 257/323/377/390, 257/377/429, 158/257/323/377/390, 257/323/377/429, 167/257/323/390/429, 377/390/429, 377/390, 257/323/377/390/429, 257/377, 390/429, 257/377/390/429, 257/390/429, 390, 243/363/390, 113/257/351/363/429, 113/257/351/390/430, 113/257/272/390, 113/243/351/390/429, 257/272/345/348, 243/257/351/390, 113/243/257/272/390, 257/272/363/390/430, 257/351/390, 113/243/390/430, 113/257/267/351/363/400/430, 113/243/257/351/430, 243/267/341/390/395, 113/243/257/345/348, 113/243/257/348/390, 243/345/348/390, 113/243/257/345/430, 257/345/348/351/390/429/430, 272/345/390/429/430, 243/267/351, 243/272/345/390, 113/257/363/390, 243/390/429, 113/243/257/351/390, 113/243/272/345/348/430, 243/267/272/348/390, 113/257/272/345/348/363/430, 113/243/390/429/430, 113/243/257/272/351/363, 243/272/345/348/363/390/395, 113/243/272/351/363/390/395, 113/243/257/272/345/390/430, 113/243/267/272/363/390/430, 113/243/267/348/363/430, 113/243/257/351/390/430, 113/243/257/272/351/390, 113/243/267/390/430, 243/257/348/395/430, 243/345/348/429/430, 243/257/345/348/395, 243/257/272/351/400/429, 113/243/267, 113/390/430, 243/267/351/400, or 429/430, wherein the amino acid positions are relative to the reference sequence corresponding to residues 13 to 468 of SEQ ID NO: 36, or relative to the reference sequence corresponding to SEQ ID NO: 36.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 238, or to the reference sequence corresponding to SEQ ID NO: 238, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 318-352, or to the reference sequence corresponding to an even-numbered SEQ ID NO. of SEQ ID NOs: 318-352, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the amino acid sequence of the engineered RNase inhibitor comprises at least a substitution or substitution set at amino acid position(s) 323/453, 323/348/452, 452/453, 323/351/430/452/453, 291, 145, 228, 323/429, 38, 150, 12, 173, 38/237, or 40, wherein the amino acid positions are relative to the reference sequence corresponding to residues 13 to 468 of SEQ ID NO: 238, or relative to the reference sequence corresponding to SEQ ID NO: 238.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 320, or to the reference sequence corresponding to SEQ ID NO: 320, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 354-390, or to the reference sequence corresponding to an even-numbered SEQ ID NO. of SEQ ID NOs: 354-390, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the amino acid sequence of the engineered RNase inhibitor comprises at least a substitution or substitution set at amino acid position(s) 13/145/228/291/348/351, 453, 12/13/453, 145/228/291/453, 13/145/453, 145/291/453, 38/145/453, 12/145/453, 38/145/228/453, 15/28/81/124/221/285/317, 15/28/64/203/221/285/317, 15/57/81/124/148/203, 12/453, 56/57/64/81/91/124/203/285/317, 66/90/142/176/205/319, 228/453, or 145/228/453, wherein the amino acid positions are relative to the reference sequence corresponding to residues 13 to 468 of SEQ ID NO: 320, or relative to the reference sequence corresponding to SEQ ID NO: 320.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 360, or to the reference sequence corresponding to SEQ ID NO: 360, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 392-554, or to the reference sequence corresponding to an even-numbered SEQ ID NO. of SEQ ID NOs: 392-554, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the amino acid sequence of the engineered RNase inhibitor comprises at least a substitution or substitution set at amino acid position(s) 319, 37, 60, 342, 81, 50, 395, 64, 177, 107, 373, 205, 35, 78, 228, 178, 285, 37/142/148/203/205/285/319, 50/81/90/124/147/148/203/205/285, 66/90/148/285/317/319, 28/37/91/142/205/285, 50/91/124/142/147/148/203/205/317/319, 50/66/90/91/124/142/285/317, 147/148/203/285/317/319, 50/66/81/90/205/319, 50/66/124/203/205/317/319, 28/66/84/90/91/142/205/319, 37/91/148/205/319, 28/66/81/124/147/285/319, 28/50/66/84/90/147/203/205/317, 28/50/90/91/124/142/319, 37/81/147/148/203/205, 28/66/81/90/91/124/203/285/317/319, 28/50/66/81/90/91/138/147/148/205/285/319, 28/37/50/124/319, 81/90/91/124/285/317, 50/90/142/203/285, 50/90/124/142/205/285/319, 28/66/90/91/147/148/205/319/326, 66/81/319, 28/66/90/203/205/319, 50/66/90/91/205, 50/66/147/205, 37/81/285, 50/66/90/91/319, 50/147/203/285, 37/50/81/148, 28/66/142/319, 15/28/66/81/90/91/147/148/319, 15/28/81/90/91/142/285/317/319, 15/28/37/50/66/124/147/148/205/285, 15/66/81/147/148, 66/91/124/142/147/148, 15/37/66/81/147/148/317/319, 15/50/90/91/124/142/147/285/317/319, 15/50/90/91/142/147/148/203/205, 15/28/81/142/147/148/285/317/319, 15/28/50/66/90/91/203/285/319, 15/66/90/91/147/148/205/285, 15/28/90/91/142/147/148/285/317/319, 15/37/91/148/285/319, 81/147/148/203/205, 147/148/285/319, 15/50/90/147/148, 15/66/81/90/285/319, 50/81/147/148/285, 37/147/148/285, 15/37/66/84/90/91/203/285/317, 15/84/90/91/203/285, 37/90/91/147, 90/91/205/317/319, 50/148/203/317, 15/28/50/66/81/84/90/285/317, 15/81/147, or 15/285, wherein the amino acid positions are relative to the reference sequence corresponding to residues 13 to 468 of SEQ ID NO: 360, or relative to the reference sequence corresponding to SEQ ID NO: 360.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 442, or to the reference sequence corresponding to SEQ ID NO: 442, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 556-724, or to the reference sequence corresponding to an even-numbered SEQ ID NO. of SEQ ID NOs: 556-724, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the amino acid sequence of the engineered RNase inhibitor comprises at least a substitution or substitution set at amino acid position(s) 78/260,396, 345, 37, 342, 395, 367, 38, 107, 228, 260, 57, 50/51, 338, 285/291/385, 175, 60, 145/147/148, 177, 28, 118, 64, 37/60/142/176/228/291/317, 60/64/317/319/342, 66/228/319/342/395, 37/142/291/317, 64/107/142/291/319/342/395, 37/60/317/319/342/395, 37/60/107/142/176/317/319/342, 142/317/319/342/395, 176/228/319/395, 228/291/342/395, 64/395, 64/107/142/176/228/319/342/395, 60/64/107/142/291/319/342, 37/66/107/291/317, 37/66/107/317/395, 37/228/317/395, 342/395, 66/142/319/342/395, 66/142/228/395, 363, 142/395, 60/64/177/291/395, 66/319, 64/107/177/228/291/317/395, 64/142/177/228/317, 60/228/317/395, 60/64/176/317/342, 177/228/342/395, 64/142/177/317/319, 60/64/107/342, 60/107/142/291/317/342/395, 66/228/319, 64/228/317/319, 168, 319/395, 37/107/228, 66/142, 37/342, 60/64/176/228/395, 60/64/342, 35, 60/66/395, 319, 291/317/319/342/395, 37/177,107, 37/60/64/142/291, 37/60/142/228/291, 60/319/342, 66/176/317/319/395, or 37/177/228, wherein the amino acid positions are relative to the reference sequence corresponding to residues 13 to 468 of SEQ ID NO: 442, or relative to the reference sequence corresponding to SEQ ID NO: 442.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 578, or to the reference sequence corresponding to SEQ ID NO: 578, wherein the amino acid sequence comprises one or more substitutions relative to the reference sequence corresponding to residues 13 to 468 of SEQ ID NO: 578, or relative to the reference sequence corresponding to SEQ ID NO: 578.

The engineered RNase inhibitor of claim 20, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 726-824, or to the reference sequence corresponding to an even-numbered SEQ ID NO. of SEQ ID NOs: 726-824, wherein the amino acid sequence comprises one or more substitutions relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least a substitution set at amino acid positions 28/118/175/260/338/367, 118/168/260/396, 38/60/168/260/338/367, 28/38/57/60/260/363/367, 57/118/260/338/367, 57/118/168/177/260/338/367, 260/396, 28/118/168/260/367/396, 28/260/338, 28/60/168/260, 57/60/118/175/177/260/338, 28/57/260/338/367, 35/260/338/367, 118/175/260/338, 168/260, 57/60/168/177/260/338/367, 28/168/260/338, 260/367, 28/57/137/260, 38/60/175/260/338, 38/60/260/367/396, 57/60/260/363/367, 60/260/367, 28/38/60/260/367, 60/168,260/363/367, 177/260/363/367, 38/260, 28/60/118/177/260, 28/35/60/118/175/260, 60/367, 28/60/118, 57/118,118/260/338/396, 28/38/57/60/118/177/260/338/363/367/396, 28/38/60/260/363/367, 28/35/38/57/118/260/363/367, 28/57/60/168/260/367, 168/175/260/338/367, 57/60/168/260/338/363/367/396, 60/168/363/367/396, 28/38/57/60/118/168/260/367, 28/175/260/367, 60/363/367/396, 260/338/367, 28/57/260/338/363/367, 168/260/367, 367/396, 28/35/175/260/367/396, or 57/60/168/260/367, wherein the amino acid positions are relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least one substitution provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or substitution set of an RNase inhibitor variant provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 13 to 468 of an engineered RNase inhibitor set forth in Tables 8.1, 9.1, 9.2, 10.1, 11.1, 12.1, 12.2, 13.1, 13.2, 14.1, and 14.2, or to the sequence of an engineered RNase inhibitor set forth in Tables 8.1, 9.1, 9.2, 10.1, 11.1, 12.1, 12.2, 13.1, 13.2, 14.1, and 14.2.

In some embodiments, the engineered RNase inhibitor comprises an amino acid sequence comprising residues 13 to 468 of an even numbered SEQ ID NO. of SEQ ID NOs: 26-824, or an amino acid sequence comprising an even numbered SEQ ID NO. of SEQ ID NOs: 26-824, optionally wherein the amino acid sequence has 1, 2, 3, 4, 5, 6, 7, 8, 9, or up to 10 substitutions.

In some embodiments, the engineered RNase inhibitor comprises an amino acid sequence comprising residues 13 to 468 of SEQ ID NO: 36, 238, 320, 360, 442, or 578, or an amino acid sequence comprising SEQ ID NO: 36, 238, 320, 360, 442, or 578, optionally wherein the amino acid sequence has 1, 2, 3, 4, 5, 6, 7, 8, 9, or up to 10 substitutions in the amino acid sequence.

In some embodiments, the engineered RNase inhibitor is characterized by at least one improved property as compared to a reference RNase inhibitor. In some embodiments, the improved property is selected from i) increased inhibitory activity against RNase A, ii) increased stability, iii) increased thermostability, iv) increased resistance to oxidation, and v) increased expression as soluble protein, or any combination of i), ii), iii), iv) and v), as compared to a reference RNase inhibitor. In some embodiments, the improved property of the engineered RNase inhibitor is in comparison to the reference RNase inhibitor having the sequence corresponding to residues 13 to 468 of SEQ ID NO: 2, 36, 238, 320, 360, 442, or 578, or the sequence corresponding to SEQ ID NO: 2, 36, 238, 320, 360, 442, or 578. In some embodiments, the reference RNase inhibitor has the sequence corresponding to residues 13 to 468 of SEQ ID NO: 2, or the sequence corresponding to SEQ ID NO: 2.

In some further embodiments, the engineered RNase inhibitor is purified. In some embodiments, the engineered RNase inhibitor is provided in solution, as a lyophilizate, or is immobilized on a substrate, such as surfaces of solid substrates, porous substrates, membranes, or particles.

In another aspect, the present disclosure provides recombinant polynucleotides encoding the engineered RNase inhibitors disclosed herein.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 37 to 1404 of an odd numbered SEQ ID NO. of SEQ ID NOs: 25-823, or to a reference polynucleotide sequence corresponding to an odd numbered SEQ ID NO. of SEQ ID NOs: 25-823, wherein the recombinant polynucleotide encodes an RNase inhibitor.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 37 to 1404 of SEQ ID NO: 35, 237, 319, 359, 441, or 577, or to a reference polynucleotide sequence corresponding to SEQ ID NO: 35, 237, 319, 359, 441, or 577, wherein the recombinant polynucleotide encodes an RNase inhibitor.

In some embodiments, the polynucleotide sequence of the recombinant polynucleotide encoding an engineered RNase inhibitor is codon optimized for expression in an organism or cell type thereof, for example a bacterial cell, fungal cell, insect cell, or mammalian cell.

In some embodiments, the recombinant polynucleotide comprises a polynucleotide sequence comprising nucleotide residues 37 to 1404 of SEQ ID NO. 35, 237, 319, 359, 441, or 577, or a polynucleotide sequence comprising SEQ ID NOs: 35, 237, 319, 359, 441, or 577.

In some embodiments, the recombinant polynucleotide comprises a polynucleotide sequence comprising nucleotide residues 37 to 1404 of an odd numbered SEQ ID NO. of SEQ ID NOs: 25-823, or a polynucleotide sequence comprising an odd numbered SEQ ID NO. of SEQ ID NOs: 25-823.

In a further aspect, the present disclosure provides expression vectors comprising at least one recombinant polynucleotide provided herein encoding an engineered RNase inhibitor. In some embodiments, the recombinant polynucleotide of the expression vector is operably linked to a control sequence. In some embodiments, the control sequence comprises a promoter, particularly a heterologous promoter.

In another aspect, the present disclosure also provides a host cell comprising at least one expression vector provided herein. In some embodiments, the host cell is a prokaryotic cell or a eukaryotic cell. In some embodiments, the host cell is a bacterial cell, fungal cell, insect cell, or mammalian cell. In some embodiments, the host cell is a bacterial cell, such as E. coli. or B. subtilis.

In a further aspect, the present disclosure provides a method of producing an engineered RNase inhibitor polypeptide, the method comprising culturing a host cell described herein under suitable culture conditions such that at least one engineered RNase inhibitor is produced. In some embodiments, the method further comprises recovering or isolating the engineered RNase inhibitor from the culture and/or host cells. In some embodiments, the method further comprises a step of purifying the engineered RNase inhibitor.

In another aspect, the present disclosure provides a composition comprising at least one engineered RNase inhibitor disclosed herein. In some embodiments, the composition comprises at least a buffer. In some embodiments, the buffer includes a reducing agent, such as dithiothreitol. In some embodiments, the composition comprises at least an RNA substrate. In some embodiments, the composition further comprises a cryoprotective agent. In some embodiments, the composition is a lyophilizate of the RNase inhibitor. In some embodiments, the composition comprises a complex of an engineered RNase inhibitor described herein and an RNase, such as RNase A.

In a further aspect, the present disclosure provides a method of inhibiting RNase, the method comprising contacting an RNase with an engineered RNase inhibitor described herein under suitable conditions for inhibiting the RNase. In some embodiments, the suitable conditions comprise a temperature of about 25° C. to about 75° C. In some embodiments, the suitable conditions comprise a temperature of 50° C. to about 75° C. In some embodiments, the RNase inhibited by the engineered RNase inhibitor is RNase A.

In some embodiments, the engineered RNase inhibitor is used in preparing or isolating a sample containing RNA. In some embodiments, the method comprises contacting a sample with an RNase inhibitor described herein. In some embodiments, the sample is a biological sample or environmental sample. In some embodiments, the sample is for conducting cDNA synthesis, RT-PCR, or in vitro RNA synthesis. In some embodiments, the sample is for isolating RNA in the sample.

In a further aspect, the present disclosure also provides a kit comprising at least one engineered RNase inhibitor disclosed herein. In some embodiments, the kit further comprises one or more of a buffer and RNA substrate. In some embodiments, the buffer comprises at least a reducing agent.

### DETAILED DESCRIPTION

The present disclosure provides engineered RNase inhibitor polypeptides and compositions thereof, as well as polynucleotides encoding the engineered RNase inhibitor polypeptides. The disclosure also provides methods of using the engineered RNase inhibitor polypeptides and compositions thereof for molecular biological, diagnostic, and other purposes. In some embodiments, the engineered RNase inhibitor polypeptides display, among others, increased activity, increased stability, and/or increased thermal stability.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, exemplary methods and materials are described herein. It is to be understood that the present invention is not limited to the particular methodology, protocols, and reagents described, as these may vary, depending upon the context they are used by those of skill in the art. Accordingly, the terms defined immediately below are more fully described by reference to the application as a whole.

As used herein, the singular “a,” “an,” and “the” include the plural references, unless the context clearly indicates otherwise.

As used herein, the term “comprising” and its cognates are used in their inclusive sense (i.e., equivalent to the term “including” and its corresponding cognates).

It is also to be understood that where description of embodiments use the term “comprising” and its cognates, the embodiments can also be described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acid” and “amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cₐ). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the xx-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“RNase inhibitor” or “RI” or “RHN1” refers to a polypeptide that inhibits the activity of an RNase. Without being bound by any theory of operation, RNase inhibitors bind to RNase and block the enzyme active site. RNase inhibitors also interact with amino acid residues in the RNase important for binding to RNA and catalysis. RNase inhibitors are generally characterized by the presence of leucine-rich repeats (LRRs), which are 20-29 residue sequence motifs present in proteins that participate in protein-protein interactions.

“RNase” or “ribonuclease” refers to nucleases capable of catalyzing the degradation of RNA. A prototypical RNase is the family of RNase A ribonucleases.

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

“Duplex” and “ds” refer to a double-stranded nucleic acid (e.g., DNA or RNA) molecule comprised of two single-stranded polynucleotides that are complementary in their sequence (A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]).

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215:403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having an alanine at the residue corresponding to X28” (or “a reference sequence corresponding to SEQ ID NO: 2, having an alanine at the residue corresponding to position 28”) refers to a reference sequence in which the corresponding residue at position X28 in SEQ ID NO: 2 (e.g., a threonine), has been changed to alanine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered RNase inhibitor, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X28 as compared to SEQ ID NO: 2” (or a “residue difference at position 28 as compared to SEQ ID NO: 2”) refers to a difference of the amino acid residue at the polypeptide position corresponding to position 28 of SEQ ID NO: 2. Thus, if the reference polypeptide of SEQ ID NO: 2 has a threonine at position 28, then a “residue difference at position X28 as compared to SEQ ID NO: 2” refers to an amino acid substitution of any residue other than threonine at the position of the polypeptide corresponding to position 28 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, where relevant, an amino acid residue difference or substitution may be a deletion and may be denoted by a “-”. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference polypeptide or amino acid sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X28A/X28H, X28A/H, or 28A/H). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant RNase inhibitor polypeptides listed in any of the Tables in the Examples. In these substitution sets, the individual substitutions are separated by a semicolon (“;”; e.g., R257F;G459E) or slash (“/”; e.g., R257F/G459E or 257F/459E).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining biological activity and/or retaining the improved properties of an engineered RNase inhibitor. Deletions can be directed to the internal portions and/or terminal portions of the polypeptide. In various embodiments, the deletion can comprise a continuous segment or can be discontinuous. As discussed herein, in some embodiments, deletions can be indicated by “-”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally-occurring or engineered polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered RNase inhibitor of the present disclosure) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant RNase inhibitor polypeptides may be present within a cell, present in the cellular medium, or prepared in various forms, such as lysates or isolated preparations. As such, in some embodiments, the recombinant RNase inhibitor polypeptides provided herein are isolated polypeptides.

“Substantially pure polypeptide” or “purified polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure RNase inhibitor composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant RNase inhibitor polypeptides are substantially pure polypeptide compositions.

“Improved property” refers to an engineered RNase inhibitor polypeptide that exhibits an improvement in any RNase inhibitor property as compared to a reference RNase inhibitor polypeptide, such as a wild-type RNase inhibitor polypeptide or another engineered RNase inhibitor polypeptide.

Improved properties include but are not limited to such properties as increased protein expression, increased thermoactivity, increased thermostability, increased stability, increased pH stability, increased inhibitory activity, increased affinity to an RNase, increased substrate range, increased chemical stability, improved solvent stability, and increased solubility.

“Increased inhibitor activity” and “enhanced inhibitor activity” refer to an improved property of the engineered RNase inhibitor polypeptides, which can be represented by an increase in specific inhibitory activity as compared to the reference RNase inhibitor polypeptide (e.g., wild-type RNase inhibitor and/or another engineered RNase inhibitor). Exemplary methods to determine inhibitory activity are provided in the Examples. Improvements in inhibitory activity can be from about 1.1 fold the inhibitory activity of the corresponding wild-type polypeptide, to about 1.5 fold, 2-fold, 5-fold, 10-fold, 20-fold, 25-fold, 50-fold, 75-fold, 100-fold, 150-fold, 200-fold or more inhibitory activity than the naturally-occurring RNase inhibitor or another engineered RNase inhibitor from which the RNase inhibitor polypeptides were derived. In some embodiments, increased inhibitor activity is reflected in increased affinity of the RNase inhibitor to an RNase.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target polynucleotide or DNA, with greater than about 90% identity to target-polynucleotide. Exemplary moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5× Denhart's solution, 5×SSPE, 0.2% SDS at 42° C., followed by washing in 0.1×SSPE, and 0.1% SDS at 65° C. Another high stringency condition comprises hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the RNase inhibitor polypeptides are codon optimized for optimal production from the host organism selected for expression.

“Control sequence” refers herein to include all components that are necessary or advantageous for the expression of a polynucleotide and/or polypeptide of the present disclosure. Each control sequence may be native or foreign (e.g., heterologous) to the nucleic acid sequence encoding the polypeptide. Such control sequences include, but are not limited to, leaders, polyadenylation sequences, propeptide sequences, promoter sequences, signal peptide sequences, initiation sequences, and transcription terminators. At a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide, and where relevant, expression of an encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” or “suitable conditions” refers to those conditions in the inhibition of RNase (e.g., temperature, pH, buffers, co-solvents, etc.) under which an RNase inhibitor polypeptide of the present disclosure is capable of inhibiting the action of RNase in degrading RNA. Exemplary “suitable conditions” are provided herein (see, the Examples).

“Culturing” refers to the growing of a population of cells under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence capable of effecting the expression in a suitable host of the polynucleotide or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one RNase inhibitor variant). In some embodiments, the host cells are prokaryotic or eukaryotic cells that have been transformed or transfected with vectors constructed using recombinant DNA techniques as known in the art.

“Analogue” in the context of a polypeptide means a polypeptide more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues include non-naturally-occurring amino acid residues including, but not limited, to homoarginine, ornithine and norvaline, as well as naturally-occurring amino acids. In some embodiments, analogues also include one or more D-amino acid residues and non-peptide linkages between two or more amino acid residues.

“Sample” as used herein refers to a material or substance for reaction with an RNase inhibitor. In some embodiments, the sample is a “biological sample,” which refers to sample of biological tissue or fluid. Such samples are typically from humans, but include tissues isolated from non-human primates, mammals, including domesticated animals (e.g., cats, dogs, pigs, cattle, horses, etc.), or rodents (e.g., mice, and rats), and includes sections of tissues such as biopsy and autopsy samples, frozen sections taken for histological purposes, blood, plasma, serum, sputum, stool, tears, mucus, hair, skin, etc. A “biological sample” also refers to a cell or population of cells or a quantity of tissue or fluid from organisms. In some embodiments, the biological sample has been removed from an animal, but the term “biological sample” can also refer to cells or tissue analyzed in vivo, i.e., without removal from the animal, including cell cultures. Typically, a “biological sample” will contain cells from the animal or of organisms, but the term can also refer to non-cellular biological material, such as non-cellular fractions of blood, saliva, or urine. Numerous types of biological samples can be used with the polypeptide, compositions, and method in the present disclosure, including, but not limited to, a tissue biopsy, a blood sample, a buccal scrape, a saliva sample, or a nipple discharge. As used herein, a “tissue biopsy” refers to an amount of tissue removed from an animal, preferably a human, for diagnostic analysis. In a patient with cancer, tissue may be removed from a tumor, allowing the analysis of cells within the tumor. “Tissue biopsy” can refer to any type of biopsy, such as needle biopsy, fine needle biopsy, surgical biopsy, etc. In some embodiments, a sample can be from environmental sources, by way of example and not limitation, water (e.g., ocean, river, refuse/sewer, etc.), soil, air, vents, or surfaces (e.g., floors, machinery, counters, etc.).

### Engineered RNase Inhibitor Polypeptides

In one aspect, the present disclosure provides RNase inhibitors, including engineered RNase inhibitor polypeptide variants. In some embodiments, the RNase inhibitors are engineered to have improved properties, including, among others, increased activity, increased stability, and increased thermostability. In some embodiments, the engineered RNase inhibitor variants find use in applications involving RNA, such as RNA isolation, RT-PCR, RNA-protein binding assays, RNA sequencing, and cDNA library synthesis.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of an even numbered SEQ ID NO. of SEQ ID NOs: 2 and 26-824, or to a reference sequence corresponding to an even numbered SEQ ID NO. of SEQ ID NOs: 2 and 26-824, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of SEQ ID NO: 2, 36, 238, 320, 360, 442, or 578, or to a reference sequence corresponding to SEQ ID NO: 2, 36, 238, 320, 360, 442, or 578, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 2, or to the reference sequence corresponding to SEQ ID NO: 2, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-142, or to a reference sequence corresponding to an even numbered SEQ ID NO. of SEQ ID NOs: 26-142, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 12K, 13T, 15R, 28A/H, 35R, 37A/C, 38A/C/R/T, 401, 50Q/S, 51H, 56A, 57M/T/V, 60K/S, 64R/V, 661, 78K/T, 81F/M/T/V, 83L, 84I/V, 90G, 91K, 94L, 95E/Q, 107A/D/I/V, 113G, 118R, 121S, 124A, 126V, 135V, 137V, 138L, 142H/Q, 145E/G/I/M/S/V, 147E, 148N, 150Q, 156M, 158C, 167K, 168F/I, 173I, 175F, 176S, 177T/V, 178D, 203E, 205I/L/P/V, 221S, 228A/L/K/R/Q, 230L, 237R, 243C/M/R/S, 257F/G/S/T/V, 260L/S/Y, 265L, 267H/K/R/T, 272N/Q, 285K/Q, 291E/S, 296I/L, 317S, 319G/T, 323E, 326L, 332L, 338V, 341C, 342A/H/M, 345A/S, 348K, 351K/R, 363E/M, 367A/L/V, 373A, 377S/T, 385A, 386G, 390G/S/T, 395C/E/I/L/M/V, 396A, 400L, 417L, 419D, 422V, 429L, 430G/L, 452K/R, 453A/F/K/L/M/R/W, 459E/K/M, or 466I, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution T12K, M13T, L15R, T28A/H, Q35R, S37A/C, E38A/C/R/T, V40I, M50Q/S, R51H, S56A, S57M/T/V, Q60K/S, S64R/V, T66I, A78K/T, H81F/M/T/V, V83L, L84I/V, P90G, T91K, 194L, R95E/Q, T107A/D/I/V, P113G, S118R, T121S, E124A, Y126V, A135V, L137V, Q138L, S142H/Q, L145E/G/I/M/S/V, P147E, Q148N, H150Q, V156M, Y158C, E167K, S168F/I, L173I, A175F, K176S, R177T/V, H178D, D203E, A205I/L/P/V, A221S, G228A/L/K/R/Q, V230L, Q237R, D243C/M/R/S, R257F/G/S/T/V, H260L/S/Y, I265L, V267H/K/R/T, V272N/Q, R285K/Q, E291P/S, M296I/L, E317S, A319G/T, Q323E, W326L, F332L, Q338V, S341C, S342A/H/M, T345A/S, T348K, V351K/R, C363E/M, Q367A/L/V, S373A, A377S/T, G385A, D386G, A390G/S/T, A395C/E/I/L/M/V, S396A, V400L, M417L, E419D, I422V, V429L, E430G/L, N452K/R, S453A/F/K/L/M/R/W, G459E/K/M, or V466I, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or amino acid residue 50Q/S, 64R/V, 81F/M/T/V, 90G, 95E, 107A/D/I/V, 113G, 124A, 142H/Q, 145E/G/I/M/S/V, 147E, 148N, 176S, 203E, 205I/P, 228A/L/K/R/Q, 243C/M/R/S, 257F/G/S/T/V, 272N/Q, 285K/Q, 291P/S, 296I/L, 319G/T, 323E, 342A/H/M, 348K, 390G/S/T, 395C/E/I/L/M/V, 452K/R, 453A/F/K/L/M/R/W, or 459E/K/M, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or amino acid residue 50S, 64R, 81T, 90G, 95E, 107D, 113G, 124A, 142Q, 145V, 147E, 148N, 176S, 203E, 205P, 228L/R, 243M, 257T, 272N, 285Q, 291P, 296L, 319G, 323E, 342M, 348K, 390G, 395L, 452R, 453R, or 459E, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 95, 296, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 95E, 296L, or 459E, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution R95E, M296L, or G459E, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 113, 243, 257, 272, or 390, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 113G, 243M, 257T, 272N, or 390G, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution P113G, D243M, R257T, D272N, or A390G, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 323, 348, or 452, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 323E, 348K, or 452R, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution Q323E, T348K, or N452R, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 145, 228, 291, or 453, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 145V, 228R, 291P, or 453R, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution L145V, G228R, E291P, or S453R, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 50, 81, 90, 124, 147, 148, 203, 205, or 285, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 50S, 81T, 90G, 124A, 147E, 148N, 203E, 205P, or 285Q, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution M50S, H81T, P90G, E124A, P147E, Q148N, D203E, A205P, or R285Q, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at amino acid position 64, 107, 142, 176, 228, 319, 342, or 395, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution 64R, 107D, 142Q, 176S, 228L, 319G, 342M, or 395L, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution S64R, T107D, S142Q, K176S, R228L, A319G, S342M, or A395L, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set at amino acid position(s) 257/459, 345, 257, 296, 113, 95/296/459, 95/257/296, 453, 107/267, 417/422/459, 395, 390, 257/296, 243, 156/257, 351, 348, 363, 95/257/296/422, 83/107/230/267/429, 107/429, 107/323/429, 94/121/265/390, 400, 429, or 81, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set 257F/459E, 345A, 257F, 296L, 113G, 95E/296L/459E, 257V, 95Q/257F/2961, 453M, 1071/267K, 417L/422V/459E, 395L, 390S, 257G/2961, 243C, 156M/257S, 351R, 348K, 363E, 453R, 95E/257F/2961/422V, 395C, 94L/121S/332L/390T/466I, 257G/417L/419D/459E, 452R, 95E/257F/2961, 377S/390T, 453F, 452K, 272N, 243S, 126V/386G, 267H, 390G, 430L, 453L, 453A, 230L/429L, 377S/390S, 257T, 459K, 272Q, 430G, 453K, 83L/1071/230L/267T/429L, 267R, 1071/429L, 243R, 243M, 107A/323E/429L, 94L/121S/265L/390T, 400L, 453W, 351K, 429L, 257F/296I, 3951, 459M, or 81F, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set R257F/G459E, T345A, R257F, M296L, P113G, R95E/M296L/G459E, R257V, R95Q/R257F/M296I, S453M, T107I/V267K, M417L/1422V/G459E, A395L, A390S, R257G/M296I, D243C, V156M/R257S, V351R, T348K, C363E, S453R, R95E/R257F/M296I/1422V, A395C, 194L/T121S/F332L/A390T/V466I, R257G/M417L/E419D/G459E, N452R, R95E/R257F/M296I, A377S/A390T, S453F, N452K, D272N, D243S, Y126V/D386G, V267H, A390G, E430L, S453L, S453A, V230L/V429L, A377S/A390S, R257T, G459K, D272Q, E430G, S453K, V83L/T107I/V230L/V267T/V429L, V267R, T107I/V429L, D243R, D243M, T107A/Q323E/V429L, 194L/T121S/1265L/A390T, V400L, S453W, V351K, V429L, R257F/M296I, A395I, G459M, or H81F, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 95/296/323/390/429/459, 95/257/296/323/429/459, 95/257/296/390/459, 95/257/296/323/390/459, 95/257/296/323/390/429/459, 95/257/296/429/459, 95/257/296/377/390/459, 95/135/257/296/323/377/390/459, 95/296/323/377/390/429/459, 95/257/296/323/390/459, 95/257/296/323/377/390/459, 95/257/296/377/429/459, 95/158/257/296/323/377/390/459, 95/257/296/323/377/429/459, 95/167/257/296/323/390/429/459, 95/296/377/390/429/459, 95/296/377/390/459, 95/257/296/323/377/390/429/459, 95/257/296/377/459, 95/296/390/429/459, 95/257/296/377/390/429/459, 95/257/296/390/429/459, or 95/296/390/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 144-222 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 95/243/296/363/390/459, 95/113/257/296/351/363/429/459, 95/113/257/296/351/390/430/459, 95/113/257/272/296/390/459, 95/113/243/296/351/390/429/459, 95/257/272/296/345/348/459, 95/243/257/296/351/390/459, 95/113/243/257/272/296/390/459, 95/257/272/296/363/390/430/459, 95/257/296/351/390/459, 95/113/243/296/390/430/459, 95/113/257/267/296/351/363/400/430/459, 95/113/243/257/296/351/430/459, 95/243/267/296/341/390/395/459, 95/113/243/257/296/345/348/459, 95/113/243/257/296/348/390/459, 95/243/296/345/348/390/459, 95/113/243/257/296/345/430/459, 95/257/296/345/348/351/390/429/430/459, 95/272/296/345/390/429/430/459, 95/243/267/296/351/459, 95/243/272/296/345/390/459, 95/113/257/296/363/390/459, 95/243/296/390/429/459, 95/113/243/257/296/351/390/459, 95/113/243/272/296/345/348/430/459, 95/243/267/272/296/348/390/459, 95/113/257/272/296/345/348/363/430/459, 95/113/243/296/390/429/430/459, 95/113/243/257/272/296/351/363/459, 95/243/272/296/345/348/363/390/395/459, 95/113/243/272/296/351/363/390/395/459, 95/113/243/257/272/296/345/390/430/459, 95/113/243/267/272/296/363/390/430/459, 95/113/243/267/296/348/363/430/459, 95/113/243/257/296/351/390/430/459, 95/113/243/257/272/296/351/390/459, 95/113/243/267/296/390/430/459, 95/243/257/296/348/395/430/459, 95/243/296/345/348/429/430/459, 95/243/257/296/345/348/395/459, 95/243/257/272/296/351/400/429/459, 95/113/243/267/296/459, 95/243/267/296/351/459, 95/113/296/390/430/459, 95/243/267/296/351/400/459, or 95/296/429/430/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 224-316 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 95/113/243/257/272/296/323/390/453/459, 95/113/243/257/272/296/323/348/390/452/459, 95/113/243/257/272/296/390/452/453/459, 95/113/243/257/272/296/323/351/390/430/452/453/459, 95/113/243/257/272/291/296/390/459, 95/113/145/243/257/272/296/390/459, 95/113/243/257/272/296/323/390/429/459, 38/95/113/243/257/272/296/390/459, 95/113/150/243/257/272/296/390/459, 95/113/228/243/257/272/296/390/459, 12/95/113/243/257/272/296/390/459, 95/113/173/243/257/272/296/390/459, 38/95/113/237/243/257/272/296/390/459, 40/95/113/243/257/272/296/390/459, or 38/95/113/243/257/272/296/390/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 318-352 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 13/95/113/145/228/243/257/272/291/296/323/351/390/452/459, 95/113/243/257/272/296/323/348/390/452/453/459, Dec. 13, 1995/113/243/257/272/296/323/348/390/452/453/459, 95/113/145/228/243/257/272/291/296/323/348/390/452/453/459, 13/95/113/145/243/257/272/296/323/348/390/452/453/459, 95/113/145/228/243/257/272/291/296/323/348/390/452/453/459, 95/113/145/243/257/272/291/296/323/348/390/452/453/459, 38/95/113/145/243/257/272/296/323/348/390/452/453/459, 12/95/113/145/243/257/272/296/323/348/390/452/453/459, 38/95/113/145/228/243/257/272/296/323/348/390/452/453/459, 15/28/81/95/113/124/221/243/257/272/285/296/317/323/348/390/452/459, 15/28/64/95/113/203/221/243/257/272/285/296/317/323/348/390/452/459, 15/57/81/95/113/124/148/203/243/257/272/296/323/348/390/452/459, 12/95/113/243/257/272/296/323/348/390/452/453/459, 56/57/64/81/91/95/113/124/203/243/257/272/285/296/317/323/348/390/452/459, 66/90/95/113/142/176/205/243/257/272/296/319/323/348/390/452/459, 95/113/228/243/257/272/296/323/348/390/452/453/459, or 95/113/145/228/243/257/272/296/323/348/390/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 354-390 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 95/113/145/228/243/257/272/291/296/319/323/348/390/452/453/459, 37/95/113/145/228/243/257/272/291/296/323/348/390/452/453/459, 60/95/113/145/228/243/257/272/291/296/323/348/390/452/453/459, 95/113/145/228/243/257/272/291/296/323/342/348/390/452/453/459, 81/95/113/145/228/243/257/272/291/296/323/348/390/452/453/459, 50/95/113/145/228/243/257/272/291/296/323/348/390/452/453/459, 95/113/145/228/243/257/272/291/296/323/348/390/395/452/453/459, 64/95/113/145/228/243/257/272/291/296/323/348/390/452/453/459, 95/113/145/177/228/243/257/272/291/296/323/348/390/452/453/459, 95/107/113/145/228/243/257/272/291/296/323/348/390/452/453/459, 50/95/113/145/228/243/257/272/291/296/323/348/390/452/453/459, 37/95/113/145/228/243/257/272/291/296/323/348/390/452/453/459, 95/113/145/228/243/257/272/291/296/323/348/373/390/452/453/459, 95/113/145/228/243/257/272/291/296/319/323/348/390/452/453/459, 95/113/145/205/228/243/257/272/291/296/323/348/390/452/453/459, 35/95/113/145/228/243/257/272/291/296/323/348/390/452/453/459, 78/95/113/145/228/243/257/272/291/296/323/348/390/452/453/459, 95/113/145/228/243/257/272/291/296/323/348/390/452/453/459, 95/113/145/178/228/243/257/272/291/296/323/348/390/452/453/459, or 95/113/145/228/243/257/272/285/291/296/323/348/390/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 392-438 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 37/95/113/142/145/148/203/205/228/243/257/272/285/291/296/319/323/348/390/452/453/459, 50/81/90/95/113/124/145/147/148/203/205/228/243/257/272/285/291/296/323/348/390/452/453/459, 66/90/95/113/145/148/228/243/257/272/285/291/296/317/319/323/348/390/452/453/459, 28/37/91/95/113/142/145/205/228/243/257/272/285/291/296/323/348/390/452/453/459, 50/91/95/113/124/142/145/147/148/203/205/228/243/257/272/291/296/317/319/323/348/390/452/453/459, 50/66/90/91/95/113/124/142/145/228/243/257/272/285/291/296/317/323/348/390/452/453/459, 95/113/145/147/148/203/228/243/257/272/285/291/296/317/319/323/348/390/452/453/459, 50/66/81/90/95/113/145/205/228/243/257/272/291/296/319/323/348/390/452/453/459, 50/66/95/113/124/145/203/205/228/243/257/272/291/296/317/319/323/348/390/452/453/459, 28/66/84/90/91/95/113/142/145/205/228/243/257/272/291/296/319/323/348/390/452/453/459, 37/91/95/113/145/148/205/228/243/257/272/291/296/319/323/348/390/452/453/459, 28/66/81/95/113/124/145/147/228/243/257/272/285/291/296/319/323/348/390/452/453/459, 28/50/66/84/90/95/113/145/147/203/205/228/243/257/272/291/296/317/323/348/390/452/453/459, 28/50/90/91/95/113/124/142/145/228/243/257/272/291/296/319/323/348/390/452/453/459, 37/81/95/113/145/147/148/203/205/228/243/257/272/291/296/323/348/390/452/453/459, 28/66/81/90/91/95/113/124/145/203/228/243/257/272/285/291/296/317/319/323/348/390/452/453/459, 28/50/66/81/90/91/95/113/138/145/147/148/205/228/243/257/272/285/291/296/319/323/348/390/452/453/459, 28/37/50/95/113/124/145/228/243/257/272/291/296/319/323/348/390/452/453/459, 81/90/91/95/113/124/145/228/243/257/272/285/291/296/317/323/348/390/452/453/459, 50/90/95/113/142/145/203/228/243/257/272/285/291/296/323/348/390/452/453/459, 50/90/95/113/124/142/145/205/228/243/257/272/285/291/296/319/323/348/390/452/453/459, 28/66/90/91/95/113/145/147/148/205/228/243/257/272/291/296/319/323/326/348/390/452/453/459, 66/81/95/113/145/228/243/257/272/291/296/319/323/348/390/452/453/459, 28/66/90/95/113/145/203/205/228/243/257/272/291/296/319/323/348/390/452/453/459, 50/66/90/91/95/113/145/205/228/243/257/272/291/296/323/348/390/452/453/459, 50/66/95/113/145/147/205/228/243/257/272/291/296/323/348/390/452/453/459, 37/81/95/113/145/228/243/257/272/285/291/296/323/348/390/452/453/459, 50/66/90/91/95/113/145/228/243/257/272/291/296/319/323/348/390/452/453/459, 50/95/113/145/147/203/228/243/257/272/285/291/296/323/348/390/452/453/459, 37/50/81/95/113/145/148/228/243/257/272/291/296/323/348/390/452/453/459, 28/66/95/113/142/145/228/243/257/272/291/296/319/323/348/390/452/453/459, 15/28/66/81/90/91/95/113/145/147/148/228/243/257/272/291/296/319/323/348/390/452/453/459, 15/28/81/90/91/95/113/142/145/228/243/257/272/285/291/296/317/319/323/348/390/452/453/459, 15/28/37/50/66/95/113/124/145/147/148/205/228/243/257/272/285/291/296/323/348/390/452/453/459, 15/66/81/95/113/145/147/148/228/243/257/272/291/296/323/348/390/452/453/459, 66/91/95/113/124/142/145/147/148/228/243/257/272/291/296/323/348/390/452/453/459, 15/37/66/81/95/113/145/147/148/228/243/257/272/291/296/317/319/323/348/390/452/453/459, 15/50/90/91/95/113/124/142/145/147/228/243/257/272/285/291/296/317/319/323/348/390/452/453/459, 15/50/90/91/95/113/142/145/147/148/203/205/228/243/257/272/291/296/323/348/390/452/453/459, 15/28/81/95/113/142/145/147/148/228/243/257/272/285/291/296/317/319/323/348/390/452/453/459, 15/28/50/66/90/91/95/113/145/203/228/243/257/272/285/291/296/319/323/348/390/452/453/459, 15/66/90/91/95/113/145/147/148/205/228/243/257/272/285/291/296/323/348/390/452/453/459, 15/28/90/91/95/113/142/145/147/148/228/243/257/272/285/291/296/317/319/323/348/390/452/453/459, 15/37/91/95/113/145/148/228/243/257/272/285/291/296/319/323/348/390/452/453/459, 81/95/113/145/147/148/203/205/228/243/257/272/291/296/323/348/390/452/453/459, 95/113/145/147/148/228/243/257/272/285/291/296/319/323/348/390/452/453/459, 15/50/90/95/113/145/147/148/228/243/257/272/291/296/323/348/390/452/453/459, 15/66/81/90/95/113/145/228/243/257/272/285/291/296/319/323/348/390/452/453/459, 50/81/95/113/145/147/148/228/243/257/272/285/291/296/323/348/390/452/453/459, 37/95/113/145/147/148/228/243/257/272/285/291/296/323/348/390/452/453/459, 15/37/66/84/90/91/95/113/145/203/228/243/257/272/285/291/296/317/323/348/390/452/453/459, 15/84/90/91/95/113/145/203/228/243/257/272/285/291/296/323/348/390/452/453/459, 37/90/91/95/113/145/147/228/243/257/272/291/296/323/348/390/452/453/459, 90/91/95/113/145/205/228/243/257/272/291/296/317/319/323/348/390/452/453/459, 50/95/113/145/148/203/228/243/257/272/291/296/317/323/348/390/452/453/459, 15/28/50/66/81/84/90/95/113/145/228/243/257/272/285/291/296/317/323/348/390/452/453/459, 15/81/95/113/145/147/228/243/257/272/291/296/323/348/390/452/453/459, or 15/95/113/145/228/243/257/272/285/291/296/323/348/390/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 440-554 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 37/50/60/81/90/95/113/124/142/145/147/148/176/203/205/228/243/257/272/285/291/296/317/323/348/390/452/453/459, 50/60/64/81/90/95/113/124/145/147/148/203/205/228/243/257/272/285/291/296/317/319/323/342/348/390/452/453/459, 50/66/81/90/95/113/124/145/147/148/203/205/228/243/257/272/285/291/296/319/323/342/348/390/395/45 2/453/459, 37/50/81/90/95/113/124/142/145/147/148/203/205/228/243/257/272/285/291/296/317/323/348/390/452/45 3/459, 50/64/81/90/95/107/113/124/142/145/147/148/203/205/228/243/257/272/285/291/296/319/323/342/348/39 0/395/452/453/459, 37/50/60/81/90/95/113/124/145/147/148/203/205/228/243/257/272/285/291/296/317/319/323/342/348/390/395/452/453/459, 37/50/60/81/90/95/107/113/124/142/145/147/148/176/203/205/228/243/257/272/285/291/296/317/319/323/342/348/390/452/453/459, 50/81/90/95/113/124/142/145/147/148/203/205/228/243/257/272/285/291/296/317/319/323/342/348/390/3 95/452/453/459, 50/81/90/95/113/124/145/147/148/176/203/205/228/243/257/272/285/291/296/319/323/348/390/395/452/4 53/459, 50/81/90/95/113/124/145/147/148/203/205/228/243/257/272/285/291/296/323/342/348/390/395/452/453/4 59, 50/64/81/90/95/113/124/145/147/148/203/205/228/243/257/272/285/291/296/323/348/390/395/452/453/45 9.

50/64/81/90/95/107/113/124/142/145/147/148/176/203/205/228/243/257/272/285/291/296/319/323/342/34 8/390/395/452/453/459, 50/81/90/95/113/124/145/147/148/203/205/228/243/257/272/285/291/296/323/348/390/395/452/453/459, 50/60/64/81/90/95/107/113/124/142/145/147/148/203/205/228/243/257/272/285/291/296/319/323/342/348/390/452/453/459, 37/50/66/81/90/95/107/113/124/145/147/148/203/205/228/243/257/272/285/291/296/317/323/348/390/452/453/459, 37/50/66/81/90/95/107/113/124/145/147/148/203/205/228/243/257/272/285/291/296/317/323/348/390/395/452/453/459, 37/50/81/90/95/113/124/145/147/148/203/205/228/243/257/272/285/291/296/317/323/348/390/395/452/45 3/459.

50/66/81/90/95/113/124/142/145/147/148/203/205/228/243/257/272/285/291/296/319/323/342/348/390/39 5/452/453/459, 50/66/81/90/95/113/124/142/145/147/148/203/205/228/243/257/272/285/291/296/323/348/390/395/452/45 3/459, 50/81/90/95/113/124/145/147/148/203/205/228/243/257/272/285/291/296/323/348/363/390/452/453/459, 50/81/90/95/113/124/142/145/147/148/203/205/228/243/257/272/285/291/296/323/348/390/395/452/453/4 59, 50/60/64/81/90/95/113/124/145/147/148/177/203/205/228/243/257/272/285/291/296/323/348/390/395/452/453/459, 50/66/81/90/95/113/124/145/147/148/203/205/228/243/257/272/285/291/296/319/323/348/390/452/453/45 9, 50/64/81/90/95/107/113/124/145/147/148/177/203/205/228/243/257/272/285/291/296/317/323/348/390/39 5/452/453/459, 50/64/81/90/95/113/124/142/145/147/148/177/203/205/228/243/257/272/285/291/296/317/323/348/390/45 2/453/459, 37/50/81/90/95/113/124/145/147/148/203/205/228/243/257/272/285/291/296/323/348/390/452/453/459, 50/81/90/95/113/124/145/147/148/203/205/228/243/257/260/272/285/291/296/323/348/390/452/453/459, 50/60/81/90/95/113/124/145/147/148/203/205/228/243/257/272/285/291/296/317/323/348/390/395/452/45 3/459, 50/60/64/81/90/95/113/124/145/147/148/176/203/205/228/243/257/272/285/291/296/317/323/342/348/390/452/453/459, 50/81/90/95/113/124/145/147/148/177/203/205/228/243/257/272/285/291/296/323/342/348/390/395/452/4 53/459, 50/64/81/90/95/113/124/142/145/147/148/177/203/205/228/243/257/272/285/291/296/317/319/323/348/39 0/452/453/459, 50/60/64/81/90/95/107/113/124/145/147/148/203/205/228/243/257/272/285/291/296/323/342/348/390/452/453/459, 50/60/81/90/95/107/113/124/142/145/147/148/203/205/228/243/257/272/285/291/296/317/323/342/348/39 0/395/452/453/459, 50/66/81/90/95/113/124/145/147/148/203/205/228/243/257/272/285/291/296/319/323/348/390/452/453/45 9, 50/64/81/90/95/113/124/145/147/148/203/205/228/243/257/272/285/291/296/317/319/323/348/390/452/45 3/459, 50/81/90/95/113/124/145/147/148/168/203/205/228/243/257/272/285/291/296/323/348/390/452/453/459, 50/81/90/95/113/124/145/147/148/203/205/228/243/257/272/285/291/296/319/323/348/390/395/452/453/4 59, 37/50/81/90/95/107/113/124/145/147/148/203/205/228/243/257/272/285/291/296/323/348/390/452/453/45 9, 50/66/81/90/95/113/124/142/145/147/148/203/205/228/243/257/272/285/291/296/323/348/390/452/453/45 9, 37/50/81/90/95/113/124/145/147/148/203/205/228/243/257/272/285/291/296/323/342/348/390/452/453/45 9, 50/60/64/81/90/95/113/124/145/147/148/176/203/205/228/243/257/272/285/291/296/323/348/390/395/452/453/459, 50/60/64/81/90/95/113/124/145/147/148/203/205/228/243/257/272/285/291/296/323/342/348/390/452/453/459, 35/50/81/90/95/113/124/145/147/148/203/205/228/243/257/272/285/291/296/323/348/390/452/453/459, 50/60/66/81/90/95/113/124/145/147/148/203/205/228/243/257/272/285/291/296/323/348/390/395/452/453/459, 50/81/90/95/113/124/145/147/148/203/205/228/243/257/272/285/291/296/319/323/348/390/452/453/459, 50/81/90/95/113/124/145/147/148/203/205/228/243/257/272/285/291/296/317/319/323/342/348/390/395/4 52/453/459, or 37/50/81/90/95/113/124/145/147/148/177/203/205/228/243/257/272/285/291/296/323/348/390/452/453/45 9, 50/81/90/95/107/113/124/145/147/148/203/205/228/243/257/272/285/291/296/323/348/390/452/453/459, 37/50/81/90/95/113/124/145/147/148/203/205/228/243/257/272/285/291/296/323/348/390/452/453/459, 37/50/60/64/81/90/95/113/124/142/145/147/148/203/205/228/243/257/272/285/291/296/323/348/390/452/453/459, 37/50/60/81/90/95/113/124/142/145/147/148/203/205/228/243/257/272/285/291/296/323/348/390/452/453/459, 50/60/81/90/95/113/124/145/147/148/203/205/228/243/257/272/285/291/296/319/323/342/348/390/452/45 3/459, or 50/66/81/90/95/113/124/145/147/148/176/203/205/228/243/257/272/285/291/296/317/319/323/348/390/39 5/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 556-666 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 50/78/81/90/95/113/124/145/147/148/203/205/228/243/257/260/272/285/291/296/323/348/390/452/453/45 9, 50/81/90/95/113/124/145/147/148/203/205/228/243/257/272/285/291/296/323/348/390/396/452/453/459, 50/81/90/95/113/124/145/147/148/203/205/228/243/257/272/285/291/296/323/345/348/390/452/453/459, 37/50/81/90/95/113/124/145/147/148/203/205/228/243/257/272/285/291/296/323/348/390/452/453/459, 50/81/90/95/113/124/145/147/148/203/205/228/243/257/272/285/291/296/323/342/348/390/452/453/459, 50/81/90/95/113/124/145/147/148/203/205/228/243/257/272/285/291/296/323/348/390/395/452/453/459, 50/81/90/95/113/124/145/147/148/203/205/228/243/257/272/285/291/296/323/348/367/390/452/453/459, 38/50/81/90/95/113/124/145/147/148/203/205/228/243/257/272/285/291/296/323/348/390/452/453/459, 50/81/90/95/107/113/124/145/147/148/203/205/228/243/257/272/285/291/296/323/348/390/452/453/459, 50/81/90/95/113/124/145/147/148/203/205/228/243/257/272/285/291/296/323/348/390/452/453/459, 50/81/90/95/113/124/145/147/148/203/205/228/243/257/260/272/285/291/296/323/348/390/452/453/459, 50/57/81/90/95/113/124/145/147/148/203/205/228/243/257/272/285/291/296/323/348/390/452/453/459, 51/81/90/95/113/124/145/147/148/203/205/228/243/257/272/285/291/296/323/348/390/452/453/459, 50/81/90/95/113/124/145/147/148/203/205/228/243/257/272/285/291/296/323/338/348/390/452/453/459, 50/81/90/95/113/124/145/147/148/203/205/228/243/257/272/296/323/348/385/390/452/453/459, 50/81/90/95/113/124/145/147/148/175/203/205/228/243/257/272/285/291/296/323/348/390/452/453/459, 50/60/81/90/95/113/124/145/147/148/203/205/228/243/257/272/285/291/296/323/348/390/452/453/459, 50/81/90/95/113/124/145/203/205/228/243/257/272/285/291/296/323/348/390/452/453/459, 50/60/81/90/95/113/124/145/147/148/203/205/228/243/257/272/285/291/296/323/348/390/452/453/459, 50/81/90/95/113/124/145/147/148/177/203/205/228/243/257/272/285/291/296/323/348/390/452/453/459, 28/50/81/90/95/113/124/145/147/148/203/205/228/243/257/272/285/291/296/323/348/390/452/453/459, 50/81/90/95/113/118/124/145/147/148/203/205/228/243/257/272/285/291/296/323/348/390/452/453/459, or 50/64/81/90/95/113/124/145/147/148/203/205/228/243/257/272/285/291/296/323/348/390/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 668-724 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 28/50/64/81/90/95/107/113/118/124/142/145/147/148/175/176/203/205/228/243/257/260/272/285/291/296/319/323/338/342/348/367/390/395/452/453/459, 50/64/81/90/95/107/113/118/124/142/145/147/148/168/176/203/205/228/243/257/260/272/285/291/296/31 9/323/342/348/390/395/396/452/453/459, 38/50/60/64/81/90/95/107/113/124/142/145/147/148/168/176/203/205/228/243/257/260/272/285/291/296/319/323/338/342/348/367/390/395/452/453/459, 28/38/50/57/60/64/81/90/95/107/113/124/142/145/147/148/176/203/205/228/243/257/260/272/285/291/29 6/319/323/342/348/363/367/390/395/452/453/459, 50/57/64/81/90/95/107/113/118/124/142/145/147/148/176/203/205/228/243/257/260/272/285/291/296/319/323/338/342/348/367/390/395/452/453/459, 50/57/64/81/90/95/107/113/118/124/142/145/147/148/168/176/177/203/205/228/243/257/260/272/285/291/296/319/323/338/342/348/367/390/395/452/453/459, 50/64/81/90/95/107/113/124/142/145/147/148/176/203/205/228/243/257/260/272/285/291/296/319/323/34 2/348/390/395/396/452/453/459, 28/50/64/81/90/95/107/113/118/124/142/145/147/148/168/176/203/205/228/243/257/260/272/285/291/296/319/323/342/348/367/390/395/396/452/453/459, 28/50/64/81/90/95/107/113/124/142/145/147/148/176/203/205/228/243/257/260/272/285/291/296/319/323/338/342/348/390/395/452/453/459, 28/50/60/64/81/90/95/107/113/124/142/145/147/148/168/176/203/205/228/243/257/260/272/285/291/296/319/323/342/348/390/395/452/453/459, 50/57/60/64/81/90/95/107/113/118/124/142/145/147/148/175/176/177/203/205/228/243/257/260/272/285/291/296/319/323/338/342/348/390/395/452/453/459, 28/50/57/64/81/90/95/107/113/124/142/145/147/148/176/203/205/228/243/257/260/272/285/291/296/319/323/338/342/348/367/390/395/452/453/459, 35/50/64/81/90/95/107/113/124/142/145/147/148/176/203/205/228/243/257/260/272/285/291/296/319/323/338/342/348/367/390/395/452/453/459, 50/64/81/90/95/107/113/118/124/142/145/147/148/175/176/203/205/228/243/257/260/272/285/291/296/31 9/323/338/342/348/390/395/452/453/459, 50/64/81/90/95/107/113/124/142/145/147/148/168/176/203/205/228/243/257/260/272/285/291/296/319/32 3/342/348/390/395/452/453/459, 50/57/60/64/81/90/95/107/113/124/142/145/147/148/168/176/177/203/205/228/243/257/260/272/285/291/296/319/323/338/342/348/367/390/395/452/453/459, 28/50/64/81/90/95/107/113/124/142/145/147/148/168/176/203/205/228/243/257/260/272/285/291/296/319/323/338/342/348/390/395/452/453/459, 50/64/81/90/95/107/113/124/142/145/147/148/176/203/205/228/243/257/260/272/285/291/296/319/323/34 2/348/367/390/395/452/453/459, 28/50/57/64/81/90/95/107/113/124/137/142/145/147/148/176/203/205/228/243/257/260/272/285/291/296/319/323/342/348/390/395/452/453/459, 38/50/60/64/81/90/95/107/113/124/142/145/147/148/175/176/203/205/228/243/257/260/272/285/291/296/319/323/338/342/348/390/395/452/453/459, 38/50/60/64/81/90/95/107/113/124/142/145/147/148/176/203/205/228/243/257/260/272/285/291/296/319/323/342/348/367/390/395/396/452/453/459, 50/57/60/64/81/90/95/107/113/124/142/145/147/148/176/203/205/228/243/257/260/272/285/291/296/319/323/342/348/363/367/390/395/452/453/459, 50/60/64/81/90/95/107/113/124/142/145/147/148/176/203/205/228/243/257/260/272/285/291/296/319/323/342/348/367/390/395/452/453/459, 28/38/50/60/64/81/90/95/107/113/124/142/145/147/148/176/203/205/228/243/257/260/272/285/291/296/3 19/323/342/348/367/390/395/452/453/459, 50/60/64/81/90/95/107/113/124/142/145/147/148/168/176/203/205/228/243/257/272/285/291/296/319/323/342/348/390/395/452/453/459, 50/64/81/90/95/107/113/124/142/145/147/148/176/203/205/228/243/257/260/272/285/291/296/319/323/34 2/348/363/367/390/395/452/453/459, 50/64/81/90/95/107/113/124/142/145/147/148/176/177/203/205/228/243/257/260/272/285/291/296/319/32 3/342/348/363/367/390/395/452/453/459, 38/50/64/81/90/95/107/113/124/142/145/147/148/176/203/205/228/243/257/260/272/285/291/296/319/323/342/348/390/395/452/453/459, 28/50/60/64/81/90/95/107/113/118/124/142/145/147/148/176/177/203/205/228/243/257/260/272/285/291/296/319/323/342/348/390/395/452/453/459, 28/35/50/60/64/81/90/95/107/113/118/124/142/145/147/148/175/176/203/205/228/243/257/260/272/285/2 91/296/319/323/342/348/390/395/452/453/459, 50/60/64/81/90/95/107/113/124/142/145/147/148/176/203/205/228/243/257/272/285/291/296/319/323/342/348/367/390/395/452/453/459, 28/50/60/64/81/90/95/107/113/118/124/142/145/147/148/176/203/205/228/243/257/272/285/291/296/319/323/342/348/390/395/452/453/459, or 50/57/64/81/90/95/107/113/118/124/142/145/147/148/176/203/205/228/243/257/272/285/291/296/319/323/342/348/390/395/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 726-790 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution set at amino acid positions 50/64/81/90/95/107/113/118/124/142/145/147/148/176/203/205/228/243/257/260/272/285/291/296/319/32 3/338/342/348/390/395/396/452/453/459, 28/38/50/57/60/64/81/90/95/107/113/118/124/142/145/147/148/176/177/203/205/228/243/257/260/272/28 5/291/296/319/323/338/342/348/363/367/390/395/396/452/453/459, 28/38/50/60/64/81/90/95/107/113/124/142/145/147/148/176/203/205/228/243/257/260/272/285/291/296/3 19/323/342/348/363/367/390/395/452/453/459, 28/35/38/50/57/64/81/90/95/107/113/118/124/142/145/147/148/176/203/205/228/243/257/260/272/285/29 1/296/319/323/342/348/363/367/390/395/452/453/459, 28/50/57/60/64/81/90/95/107/113/124/142/145/147/148/168/176/203/205/228/243/257/260/272/285/291/2 96/319/323/342/348/367/390/395/452/453/459, 50/64/81/90/95/107/113/124/142/145/147/148/168/175/176/203/205/228/243/257/260/272/285/291/296/31 9/323/338/342/348/367/390/395/452/453/459, 50/57/60/64/81/90/95/107/113/124/142/145/147/148/168/176/203/205/228/243/257/260/272/285/291/296/319/323/338/342/348/363/367/390/395/396/452/453/459, 50/60/64/81/90/95/107/113/124/142/145/147/148/168/176/203/205/228/243/257/272/285/291/296/319/323/342/348/363/367/390/395/396/452/453/459, 28/38/50/57/60/64/81/90/95/107/113/118/124/142/145/147/148/168/176/203/205/228/243/257/260/272/28 5/291/296/319/323/342/348/367/390/395/452/453/459, 28/50/64/81/90/95/107/113/124/142/145/147/148/175/176/203/205/228/243/257/260/272/285/291/296/319/323/342/348/367/390/395/452/453/459, 50/60/64/81/90/95/107/113/124/142/145/147/148/176/203/205/228/243/257/272/285/291/296/319/323/342/348/363/367/390/395/396/452/453/459, 50/64/81/90/95/107/113/124/142/145/147/148/176/203/205/228/243/257/260/272/285/291/296/319/323/33 8/342/348/367/390/395/452/453/459, 28/50/57/64/81/90/95/107/113/124/142/145/147/148/176/203/205/228/243/257/260/272/285/291/296/319/323/338/342/348/363/367/390/395/452/453/459, 50/64/81/90/95/107/113/124/142/145/147/148/168/176/203/205/228/243/257/260/272/285/291/296/319/32 3/342/348/367/390/395/452/453/459, 50/64/81/90/95/107/113/124/142/145/147/148/176/203/205/228/243/257/272/285/291/296/319/323/342/34 8/367/390/395/396/452/453/459, 28/35/50/64/81/90/95/107/113/124/142/145/147/148/175/176/203/205/228/243/257/260/272/285/291/296/319/323/342/348/367/390/395/396/452/453/459, or 50/57/60/64/81/90/95/107/113/124/142/145/147/148/168/176/203/205/228/243/257/260/272/285/291/296/319/323/342/348/367/390/395/452/453/459, wherein the amino acid positions are relative to the reference sequence corresponding to residues 13 to 468 of SEQ ID NO: 2, or relative to the reference sequence corresponding to SEQ ID NO: 2. In some embodiments, the amino acid sequence of the engineered RNase inhibitor comprises at least a substitution or substitution set of an even numbered SEQ ID NO. of SEQ ID NOs: 792-824 set forth in the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution at an amino acid position provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least one substitution provided in Tables 8.1, 9.1, 9.2, 10.1, 11.1, 12.1, 12.2, 13.1, 13.2, 14.1, 14.2, and

Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set at the amino acid position(s) provided in Tables 8.1, 9.1, 9.2, 10.1, 11.1, 12.1, 12.2, 13.1, 13.2, 14.1, 14.2, and Appendix, wherein the amino acid positions are relative to the reference sequence corresponding to residues 13 to 468 of SEQ ID NO: 2, or relative to the reference sequence corresponding to SEQ ID NO: 2.

In some embodiments, the amino acid sequence of the engineered RNase inhibitor comprises at least a substitution or substitution set of an RNase inhibitor variant provided in Tables 8.1, 9.1, 9.2, 10.1, 11.1, 12.1, 12.2, 13.1, 13.2, 14.1, 14.2, and Appendix, wherein the amino acid positions are relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set provided in Tables 8.1, 9.1, 9.2, 10.1, 11.1, 12.1, 12.2, 13.1, 13.2, 14.1, 14.2, and Appendix, wherein the amino acid positions are relative to the reference sequence corresponding to residues 13 to 468 of SEQ ID NO: 2, or relative to the reference sequence corresponding to SEQ ID NO: 2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to a reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or an amino acid residue 12K, 13T, 15R, 28A/H, 35R, 37A/C, 38A/C/R/T, 40I, 50Q/S/M, 51H, 56A, 57M/T/V, 60K/S, 64R/V, 661, 78K/T, 81F/M/T/V, 83L, 84I/V, 90G, 91K, 94L, 95E/Q, 107A/D/I/V, 113G, 118R, 121S, 124A, 126V, 135V, 137V, 138L, 142H/Q, 145E/G/I/M/S/V, 147E/P, 148Q/N, 150Q, 156M, 158C, 167K, 168F/I, 1731, 175F, 176S, 177T/V, 178D, 203E, 205I/L/P/V, 221S, 228A/L/K/R/Q, 230L, 237R, 243C/M/R/S, 257F/G/S/T/V, 260L/S/Y, 265L, 267H/K/R/T, 272N/Q, 285K/Q, 291E/P/S, 296I/L, 317S, 319G/T, 323E, 326L, 332L, 338V, 341C, 342A/H/M, 345A/S, 348T/K, 351K/R, 363E/M, 367A/L/V, 373A, 377S/T, 385A, 386G, 390G/S/T, 395C/E/I/L/M/V, 396A, 400L, 417L, 419D, 422V, 429L, 430G/L, 452K/R, 453A/F/K/L/M/R/W, 459E/K/M, or 466I, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or an amino acid residue 50Q/S/M, 60K/S, 81F/M/T/V, 90G, 95E/Q, 107A/D/I/V, 113G, 124A, 142H/Q, 145E/G/I/M/S/V, 147E/P, 148Q/N, 176S, 203E, 205I/L/P/V, 228A/L/K/R/Q, 243C/M/R/S, 257F/G/S/T/V, 272N/Q, 285K/Q, 291E/P/S, 296I/L, 319G/T, 323E, 342A/H/M, 348T/K, 390G/S/T, 395C/E/I/L/M/V, 452K/R, 453A/F/K/L/M/R/W, or 459E/K/M, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or an amino acid residue 50S, 64R, 81T, 90G, 95E, 107D, 113G, 124A, 142Q, 145V, 147E, 148N, 176S, 203E, 205P, 228R, 228L, 243M, 257T, 272N, 285Q, 291P, 296L, 319G, 323E, 342M, 348K, 390G, 395L, 452R, 453R, or 459E, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution at amino acid position 95, 296, or 459, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or an amino acid residue 95E, 296L, or 459E, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, or to the reference sequence corresponding to SEQ ID NO: 36, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 144-316, or to the reference sequence corresponding to an even-numbered SEQ ID NO. of SEQ ID NOs: 144-316, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the amino acid sequence of the engineered RNase inhibitors comprises at least a substitution or substitution set at amino acid positions(s) 323/390/429, 257/323/429, 257/390, 257/323/390, 257/323/390/429, 257/429, 257/377/390, 135/257/323/377/390, 323/377/390/429, 257/323/377/390, 257/377/429, 158/257/323/377/390, 257/323/377/429, 167/257/323/390/429, 377/390/429, 377/390, 257/323/377/390/429, 257/377, 390/429, 257/377/390/429, 257/390/429, 390, 243/363/390, 113/257/351/363/429, 113/257/351/390/430, 113/257/272/390, 113/243/351/390/429, 257/272/345/348, 243/257/351/390, 113/243/257/272/390, 257/272/363/390/430, 257/351/390, 113/243/390/430, 113/257/267/351/363/400/430, 113/243/257/351/430, 243/267/341/390/395, 113/243/257/345/348, 113/243/257/348/390, 243/345/348/390, 113/243/257/345/430, 257/345/348/351/390/429/430, 272/345/390/429/430, 243/267/351, 243/272/345/390, 113/257/363/390, 243/390/429, 113/243/257/351/390, 113/243/272/345/348/430, 243/267/272/348/390, 113/257/272/345/348/363/430, 113/243/390/429/430, 113/243/257/272/351/363, 243/272/345/348/363/390/395, 113/243/272/351/363/390/395, 113/243/257/272/345/390/430, 113/243/267/272/363/390/430, 113/243/267/348/363/430, 113/243/257/351/390/430, 113/243/257/272/351/390, 113/243/267/390/430, 243/257/348/395/430, 243/345/348/429/430, 243/257/345/348/395, 243/257/272/351/400/429, 113/243/267, 113/390/430, 243/267/351/400, or 429/430, wherein the amino acid positions are relative to the reference sequence corresponding to residues 13 to 468 of SEQ ID NO: 36, or relative to the reference sequence corresponding to SEQ ID NO: 36.

In some embodiments, the amino acid sequence of the engineered RNase inhibitors comprises at least a substitution or substitution set 323E/390S/429L, 257F/323E/429L, 257F/390S, 257F/323E/390T, 257F/323E/390S/429L, 257F/429L, 257F/377T/390T, 135V/257F/323E/377S/390T, 323E/377T/390S/429L, 257F/323E/390S, 257F/323E/377S/390S, 257F/323E/377T/390T, 257F/377S/429L, 257F/390T, 158C/257F/323E/377T/390S, 257F/377T/429L, 257F/377S/390S, 257F/323E/377S/390T, 257F/323E/377T/429L, 167K/257F/323E/390S/429L, 323E/390T/429L, 257F/323E/390T/429L, 377T/390S/429L, 257F/377T/390S, 377T/390S, 257F/323E/377S/390S/429L, 257F/323E/377S/390T/429L, 257F/323E/377S/429L, 257F/377S, 390T/429L, 257F/323E/377T/390S, 257F/323E/377T/390S/429L, 257F/377T/390T/429L, 257F/390T/429L, 257F/377T/390S/429L, 257F/377S/390T, 257F/377S/390T/429L, 257F/390S/429L, 377S/390T, 390T, 243S/363E/390G, 113G/257F/351K/363E/429L, 113G/257V/351K/390S/430G, 113G/257V/272N/390S, 113G/243M/351K/390S/429L, 257V/272N/345A/348K, 243S/257T/351R/390S, 113G/243M/257T/272N/390G, 257T/272Q/363E/390G/430L, 257V/351K/390G, 113G/243S/390G/430G, 113G/257V/267H/351R/363E/400L/430L, 113G/243M/257V/351R/430G, 243M/267R/341C/390G/395L, 113G/243M/257T/345A/348K, 113G/243S/257F/348K/390G, 243S/345A/348K/390G, 113G/243M/257V/345A/430L, 257T/345A/348K/351R/390S/429L/430G, 272Q/345A/390G/429L/430L, 243M/267R/351K, 243R/272N/345A/390G, 113G/257T/363E/390G, 243S/390G/429L, 113G/243M/257F/351K/390G, 113G/243M/272Q/345A/348K/430G, 243M/267R/272Q/348K/390G, 113G/257T/272Q/345A/348K/363E/430L, 113G/243R/390G/429L/430L, 113G/243M/257F/272N/351R/363E, 243M/272Q/345A/348K/363E/390S/395L, 113G/243M/272Q/351R/363E/390S/395L, 113G/243R/257V/272Q/345A/390S/430L, 113G/243S/267R/272Q/363E/390S/430G, 113G/243M/267R/348K/363E/430L, 113G/243S/257T/351R/390G/430L, 113G/243S/257T/272N/351K/390S, 113G/243R/267R/390G/430G, 243R/257F/348K/395L/430G, 243R/345A/348K/429L/430L, 243M/257F/345A/348K/395L, 243S/257T/272N/351K/400L/429L, 113G/243M/267R, 243M/267R/351R, 113G/390S/430G, 243S/267R/351K/400L, or 429L/430G, wherein the amino acid positions are relative to the reference sequence corresponding to residues 13 to 468 of SEQ ID NO: 36, or relative to the reference sequence corresponding to SEQ ID NO: 36.

In some embodiments, the amino acid sequence of the engineered RNase inhibitors comprises at least a substitution or substitution set Q323E/A390S/V429L, R257F/Q323E/V429L, R257F/A390S, R257F/Q323E/A390T, R257F/Q323E/A390S/V429L, R257F/V429L, R257F/A377T/A390T, A135V/R257F/Q323E/A377S/A390T, Q323E/A377T/A390S/V429L, R257F/Q323E/A390S, R257F/Q323E/A377S/A390S, R257F/Q323E/A377T/A390T, R257F/A377S/V429L, R257F/A390T, Y158C/R257F/Q323E/A377T/A390S, R257F/A377T/V429L, R257F/A377S/A390S, R257F/Q323E/A377S/A390T, R257F/Q323E/A377T/V429L, E167K/R257F/Q323E/A390S/V429L, Q323E/A390T/V429L, R257F/Q323E/A390T/V429L, A377T/A390S/V429L, R257F/A377T/A390S, A377T/A390S, R257F/Q323E/A377S/A390S/V429L, R257F/Q323E/A377S/A390T/V429L, R257F/Q323E/A377S/V429L, R257F/A377S, A390T/V429L, R257F/Q323E/A377T/A390S, R257F/Q323E/A377T/A390S/V429L, R257F/A377T/A390T/V429L, R257F/A390T/V429L, R257F/A377T/A390S/V429L, R257F/A377S/A390T, R257F/A377S/A390T/V429L, R257F/A390S/V429L, A377S/A390T, A390T, D243S/C363E/A390G,

P113G/R257F/V351K/C363E/V429L, P113G/R257V/V351K/A390S/E430G,

P113G/R257V/D272N/A390S, P113G/D243M/V351K/A390S/V429L, R257V/D272N/T345A/T348K, D243S/R257T/V351R/A390S, P113G/D243M/R257T/D272N/A390G, R257T/D272Q/C363E/A390G/E430L, R257V/V351K/A390G, P113G/D243S/A390G/E430G,

P113G/R257V/V267H/V351R/C363E/V400L/E430L, P113G/D243M/R257V/V351R/E430G, D243M/V267R/S341C/A390G/A395L, P113G/D243M/R257T/T345A/T348K, P113G/D243S/R257F/T348K/A390G, D243S/T345A/T348K/A390G, P113G/D243M/R257V/T345A/E430L, R257T/T345A/T348K/V351R/A390S/V429L/E430G, D272Q/T345A/A390G/V429L/E430L, D243M/V267R/V351K, D243R/D272N/T345A/A390G, P113G/R257T/C363E/A390G, D243S/A390G/V429L, P113G/D243M/R257F/V351K/A390G, P113G/D243M/D272Q/T345A/T348K/E430G, D243M/V267R/D272Q/T348K/A390G, P113G/R257T/D272Q/T345A/T348K/C363E/E430L, P113G/D243R/A390G/V429L/E430L, P113G/D243M/R257F/D272N/V351R/C363E, D243M/D272Q/T345A/T348K/C363E/A390S/A395L, P113G/D243M/D272Q/V351R/C363E/A390S/A395L,

P113G/D243R/R257V/D272Q/T345A/A390S/E430L,

P113G/D243S/V267R/D272Q/C363E/A390S/E430G, P113G/D243M/V267R/T348K/C363E/E430L, P113G/D243S/R257T/V351R/A390G/E430L, P113G/D243S/R257T/D272N/V351K/A390S, P113G/D243R/V267R/A390G/E430G, D243R/R257F/T348K/A395L/E430G, D243R/T345A/T348K/V429L/E430L, D243M/R257F/T345A/T348K/A395L, D243S/R257T/D272N/V351K/V400L/V429L, P113G/D243M/V267R, D243M/V267R/V351R, P113G/A390S/E430G, D243S/V267R/V351K/V400L, or V429L/E430G, wherein the amino acid positions are relative to the reference sequence corresponding to residues 13 to 468 of SEQ ID NO: 36, or relative to the reference sequence corresponding to SEQ ID NO: 36.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 238, or to the reference sequence corresponding to SEQ ID NO: 238, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 318-352, or to the reference sequence corresponding to an even-numbered SEQ ID NO. of SEQ ID NOs: 318-352, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the amino acid sequence of the engineered RNase inhibitors comprises at least a substitution or substitution set at amino acid positions(s) 323/453, 323/348/452, 452/453, 323/351/430/452/453, 291, 145, 228, 323/429, 38, 150, 12, 173, 38/237, or 40, wherein the amino acid positions are relative to the reference sequence corresponding to residues 13 to 468 of SEQ ID NO: 238, or relative to the reference sequence corresponding to SEQ ID NO: 238.

In some embodiments, the amino acid sequence of the engineered RNase inhibitors comprises at least a substitution or substitution set 323E/453K, 323E/348K/452R, 452R/453A, 323E/351R/430L/452R/453A, 291P, 145E, 228A, 323E/429L, 145G, 145M, 38T, 150Q, 228R, 12K, 173I, 38C/237R, 40I, or 38R, wherein the amino acid positions are relative to the reference sequence corresponding to residues 13 to 468 of SEQ ID NO: 238, or relative to the reference sequence corresponding to SEQ ID NO: 238.

In some embodiments, the amino acid sequence of the engineered RNase inhibitors comprises at least a substitution or substitution set Q323E/S453K, Q323E/T348K/N452R, N452R/S453A, Q323E/V351R/E430L/N452R/S453A, E291P, L145E, G228A, Q323E/V429L, L145G, L145M, E38T, H150Q, G228R, T12K, L173I, E38C/Q237R, V40I, or E38R, wherein the amino acid positions are relative to the reference sequence corresponding to residues 13 to 468 of SEQ ID NO: 238, or relative to the reference sequence corresponding to SEQ ID NO: 238.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 320, or to the reference sequence corresponding to SEQ ID NO: 320, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 354-390, or to the reference sequence corresponding to an even-numbered SEQ ID NO. of SEQ ID NOs: 354-390, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the amino acid sequence of the engineered RNase inhibitor comprises at least a substitution or substitution set at amino acid position(s) 13/145/228/291/348/351,453, 12/13/453, 145/228/291/453, 13/145/453,145/291/453, 38/145/453, 12/145/453, 38/145/228/453, 15/28/81/124/221/285/317, 15/28/64/203/221/285/317, 15/57/81/124/148/203,12/453, 56/57/64/81/91/124/203/285/317, 66/90/142/176/205/319, 228/453, or 145/228/453, wherein the amino acid positions are relative to the reference sequence corresponding to residues 13 to 468 of SEQ ID NO: 320, or relative to the reference sequence corresponding to SEQ ID NO: 320.

In some embodiments, the amino acid sequence of the engineered RNase inhibitor comprises at least a substitution or substitution set 13T/145M/228A/291P/348T/351R, 453R, 12K/13T/453R, 145V/228R/291P/453R, 13T/145V/453R, 145M/228R/291P/453R, 145V/291P/453R, 38T/145V/453R, 12K/145M/453R, 453K, 38T/1451/228A/453R, 15R/28H/81T/124A/221S/285Q/317S, 15R/28H/64V/203E/221S/285Q/317S, 15R/57M/81T/124A/148N/203E, 12K/453K, 56A/57T/64V/81T/91K/124A/203E/285Q/317S, 661/90G/142H/176S/205P/319G, 228R/453K, or 1451/228A/453A, wherein the amino acid positions are relative to the reference sequence corresponding to residues 13 to 468 of SEQ ID NO: 320, or relative to the reference sequence corresponding to SEQ ID NO: 320.

In some embodiments, the amino acid sequence of the engineered RNase inhibitor comprises at least a substitution or substitution set M13T/L145M/G228A/E291P/K348T/V351R, S453R, T12K/M13T/S453R, L145V/G228R/E291P/S453R, M13T/L145V/S453R, L145M/G228R/E291P/S453R, L145V/E291P/S453R, E38T/L145V/S453R, T12K/L145M/S453R, S453K, E38T/L145I/G228A/S453R, L15R/T28H/H81T/E124A/A221S/R285Q/E317S, L15R/T28H/S64V/D203E/A221S/R285Q/E317S, L15R/S57M/H81T/E124A/Q148N/D203E, T12K/S453K, S56A/S57T/S64V/H81T/T91K/E124A/D203E/R285Q/E317S, T66I/P90G/S142H/K176S/A205P/A319G, G228R/S453K, or L145I/G228A/S453A, wherein the amino acid positions are relative to the reference sequence corresponding to residues 13 to 468 of SEQ ID NO: 320, or relative to the reference sequence corresponding to SEQ ID NO: 320.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 360, or to the reference sequence corresponding to SEQ ID NO: 360, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 392-554, or to the reference sequence corresponding to an even-numbered SEQ ID NO. of SEQ ID NOs: 392-554, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the amino acid sequence of the engineered RNase inhibitor comprises at least a substitution or substitution set at amino acid position(s) 319, 37, 60, 342, 81, 50, 395, 64, 177, 107, 373, 205, 35, 78, 228, 178, 285, 37/142/148/203/205/285/319, 50/81/90/124/147/148/203/205/285, 66/90/148/285/317/319, 28/37/91/142/205/285, 50/91/124/142/147/148/203/205/317/319, 50/66/90/91/124/142/285/317, 147/148/203/285/317/319, 50/66/81/90/205/319, 50/66/124/203/205/317/319, 28/66/84/90/91/142/205/319, 37/91/148/205/319, 28/66/81/124/147/285/319, 28/50/66/84/90/147/203/205/317, 28/50/90/91/124/142/319, 37/81/147/148/203/205, 28/66/81/90/91/124/203/285/317/319, 28/50/66/81/90/91/138/147/148/205/285/319, 28/37/50/124/319, 81/90/91/124/285/317, 50/90/142/203/285, 50/90/124/142/205/285/319, 28/66/90/91/147/148/205/319/326, 66/81/319, 28/66/90/203/205/319, 50/66/90/91/205, 50/66/147/205, 37/81/285, 50/66/90/91/319, 50/147/203/285, 37/50/81/148, 28/66/142/319, 15/28/66/81/90/91/147/148/319, 15/28/81/90/91/142/285/317/319, 15/28/37/50/66/124/147/148/205/285, 15/66/81/147/148, 66/91/124/142/147/148, 15/37/66/81/147/148/317/319, 15/50/90/91/124/142/147/285/317/319, 15/50/90/91/142/147/148/203/205, 15/28/81/142/147/148/285/317/319, 15/28/50/66/90/91/203/285/319, 15/66/90/91/147/148/205/285, 15/28/90/91/142/147/148/285/317/319, 15/37/91/148/285/319, 81/147/148/203/205, 147/148/285/319, 15/50/90/147/148, 15/66/81/90/285/319, 50/81/147/148/285, 37/147/148/285, 15/37/66/84/90/91/203/285/317, 15/84/90/91/203/285, 37/90/91/147, 90/91/205/317/319, 50/148/203/317, 15/28/50/66/81/84/90/285/317, 15/81/147, or 15/285, wherein the amino acid positions are relative to the reference sequence corresponding to residues 13 to 468 of SEQ ID NO: 360, or relative to the reference sequence corresponding to SEQ ID NO: 360.

In some embodiments, the amino acid sequence of the engineered RNase inhibitor comprises at least a substitution or substitution set 319T, 37C, 60K, 342A, 342M, 81V, 81M, 50S, 395L, 64R, 177T, 107D, 50Q, 37A, 373A, 319G, 205L, 2051, 205V, 35R, 78K, 228L, 178D, 285K, 37A/142Q/148N/203E/205P/285Q/319G, 50S/81T/90G/124A/147E/148N/203E/205P/285Q, 661/90G/148N/285Q/317S/319G, 28H/37A/91K/142Q/205P/285Q, 50S/91K/124A/142Q/147E/148N/203E/205P/317S/319G, 50S/661/90G/91K/124A/142Q/285Q/317S, 147E/148N/203E/285Q/317S/319G, 50S/661/81T/90G/205P/319G, 50S/66I/124A/203E/205P/317S/319G, 28H/661/841/90G/91K/142Q/205P/319G, 37A/91K/148N/205P/319G, 28H/661/81T/124A/147E/285Q/319G, 28H/50S/661/841/90G/147E/203E/205P/317S, 28H/50S/90G/91K/124A/142Q/319G, 37A/81T/147E/148N/203E/205P, 28H/66I/81T/90G/91K/124A/203E/285Q/317S/319G, 28H/50S/66I/81T/90G/91K/138L/147E/148N/205P/285Q/319G, 28H/37A/50S/124A/319G, 81T/90G/91K/124A/285Q/317S, 50S/90G/142Q/203E/285Q, 50S/90G/124A/142Q/205P/285Q/319G, 28H/661/90G/91K/147E/148N/205P/319G/326L, 661/81T/319G, 28H/66I/90G/203E/205P/319G, 50S/66I/90G/91K/205P, 50S/66I/147E/205P, 37A/81T/285Q, 50S/661/90G/91K/319G, 50S/147E/203E/285Q, 37A/50S/81T/148N, 28H/66I/142Q/319G, 15R/28H/66I/81T/90G/91K/147E/148N/319G, 15R/28H/81T/90G/91K/142Q/285Q/317S/319G, 15R/28H/37A/50S/66I/124A/147E/148N/205P/285Q, 15R/661/81T/147E/148N, 661/91K/124A/142Q/147E/148N, 15R/37A/66I/81T/147E/148N/317S/319G, 15R/50S/90G/91K/124A/142Q/147E/285Q/317S/319G, 15R/50S/90G/91K/142Q/147E/148N/203E/205P, 15R/28H/81T/142Q/147E/148N/285Q/317S/319G, 15R/28H/50S/661/90G/91K/203E/285Q/319G, 15R/66I/90G/91K/147E/148N/205P/285Q, 15R/28H/90G/91K/142Q/147E/148N/285Q/317S/319G, 15R/37A/91K/148N/285Q/319G, 81T/147E/148N/203E/205P, 147E/148N/285Q/319G, 15R/50S/90G/147E/148N, 15R/66I/81T/90G/285Q/319G, 50S/81T/147E/148N/285Q, 37A/147E/148N/285Q, 15R/37A/66I/84V/90G/91K/203E/285Q/317S, 15R/841/90G/91K/203E/285Q, 37A/90G/91K/147E, 90G/91K/205P/317S/319G, 50S/148N/203E/317S, 15R/28H/50S/661/81T/841/90G/285Q/317S, 15R/81T/147E, or 15R/285Q, wherein the amino acid positions are relative to the reference sequence corresponding to residues 13 to 468 of SEQ ID NO: 360, or relative to the reference sequence corresponding to SEQ ID NO: 360.

In some embodiments, the amino acid sequence of the engineered RNase inhibitor comprises at least a substitution or substitution set A319T, S37C, Q60K, S342A, S342M, H81V, H81M, M50S, A395L, S64R, R177T, T107D, M50Q, S37A, S373A, A319G, A205L, A2051, A205V, Q35R, A78K, R228L, H178D, R285K, S37A/S142Q/Q148N/D203E/A205P/R285Q/A319G, M50S/H81T/P90G/E124A/P147E/Q148N/D203E/A205P/R285Q, T66I/P90G/Q148N/R285Q/E317S/A319G, T28H/S37A/T91K/S142Q/A205P/R285Q, M50S/T91K/E124A/S142Q/P147E/Q148N/D203E/A205P/E317S/A319G, M50S/T66I/P90G/T91K/E124A/S142Q/R285Q/E317S, P147E/Q148N/D203E/R285Q/E317S/A319G, M50S/T66I/H81T/P90G/A205P/A319G, M50S/T66I/E124A/D203E/A205P/E317S/A319G, T28H/T66I/L84I/P90G/T91K/S142Q/A205P/A319G, S37A/T91K/Q148N/A205P/A319G, T28H/T66I/H81T/E124A/P147E/R285Q/A319G,

T28H/M50S/T66I/L84I/P90G/P147E/D203E/A205P/E317S, T28H/M50S/P90G/T91K/E124A/S142Q/A319G, S37A/H81T/P147E/Q148N/D203E/A205P, T28H/T66I/H81T/P90G/T91K/E124A/D203E/R285Q/E317S/A319G,

T28H/M50S/T66I/H81T/P90G/T91K/Q138L/P147E/Q148N/A205P/R285Q/A319G, T28H/S37A/M50S/E124A/A319G, H81T/P90G/T91K/E124A/R285Q/E317S, M50S/P90G/S142Q/D203E/R285Q, M50S/P90G/E124A/S142Q/A205P/R285Q/A319G,

T28H/T66I/P90G/T91K/P147E/Q148N/A205P/A319G/W326L, T66I/H81T/A319G, T28H/T66I/P90G/D203E/A205P/A319G, M50S/T66I/P90G/T91K/A205P, M50S/T66I/P147E/A205P, S37A/H81T/R285Q, M50S/T66I/P90G/T91K/A319G, M50S/P147E/D203E/R285Q, S37A/M50S/H81T/Q148N, T28H/T66I/S142Q/A319G,

L15R/T28H/T66I/H81T/P90G/T91K/P147E/Q148N/A319G, L15R/T28H/H81T/P90G/T91K/S142Q/R285Q/E317S/A319G,

L15R/T28H/S37A/M50S/T66I/E124A/P147E/Q148N/A205P/R285Q, L15R/T66I/H81T/P147E/Q148N, T66I/T91K/E124A/S142Q/P147E/Q148N, L15R/S37A/T66I/H81T/P147E/Q148N/E317S/A319G,

L15R/M50S/P90G/T91K/E124A/S142Q/P147E/R285Q/E317S/A319G,

L15R/M50S/P90G/T91K/S142Q/P147E/Q148N/D203E/A205P, L15R/T28H/H81T/S142Q/P147E/Q148N/R285Q/E317S/A319G, L15R/T28H/M50S/T66I/P90G/T91K/D203E/R285Q/A319G, L15R/T66I/P90G/T91K/P147E/Q148N/A205P/R285Q, L15R/T28H/P90G/T91K/S142Q/P147E/Q148N/R285Q/E317S/A319G, L15R/S37A/T91K/Q148N/R285Q/A319G, H81T/P147E/Q148N/D203E/A205P, P147E/Q148N/R285Q/A319G, L15R/M50S/P90G/P147E/Q148N, L15R/T66I/H81T/P90G/R285Q/A319G, M50S/H81T/P147E/Q148N/R285Q, S37A/P147E/Q148N/R285Q, L15R/S37A/T66I/L84V/P90G/T91K/D203E/R285Q/E317S, L15R/L84I/P90G/T91K/D203E/R285Q, S37A/P90G/T91K/P147E, P90G/T91K/A205P/E317S/A319G, M50S/Q148N/D203E/E317S, L15R/T28H/M50S/T66I/H81T/L84I/P90G/R285Q/E317S, L15R/H81T/P147E, or L15R/R285Q, wherein the amino acid positions are relative to the reference sequence corresponding to residues 13 to 468 of SEQ ID NO: 360, or relative to the reference sequence corresponding to SEQ ID NO: 360.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 442, or to the reference sequence corresponding to SEQ ID NO: 442, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 556-724, or to the reference sequence corresponding to an even-numbered SEQ ID NO. of SEQ ID NOs: 556-724, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the amino acid sequence of the engineered RNase inhibitor comprises at least a substitution or substitution set at amino acid position(s) 78/260,396, 345, 37, 342, 395, 367, 38, 107, 228, 260, 57, 50/51, 338, 285/291/385, 175, 60, 145/147/148, 177, 28, 118, 64, 37/60/142/176/228/291/317, 60/64/317/319/342, 66/228/319/342/395, 37/142/291/317, 64/107/142/291/319/342/395, 37/60/317/319/342/395, 37/60/107/142/176/317/319/342, 142/317/319/342/395, 176/228/319/395, 228/291/342/395, 64/395, 64/107/142/176/228/319/342/395, 60/64/107/142/291/319/342, 37/66/107/291/317, 37/66/107/317/395, 37/228/317/395, 342/395, 66/142/319/342/395, 66/142/228/395, 363, 142/395, 60/64/177/291/395, 66/319, 64/107/177/228/291/317/395, 64/142/177/228/317, 60/228/317/395, 60/64/176/317/342, 177/228/342/395, 64/142/177/317/319, 60/64/107/342, 60/107/142/291/317/342/395, 66/228/319, 64/228/317/319, 168, 319/395, 37/107/228, 66/142, 37/342, 60/64/176/228/395, 60/64/342, 35, 60/66/395, 319, 291/317/319/342/395, 37/177,107, 37/60/64/142/291, 37/60/142/228/291, 60/319/342, 66/176/317/319/395, or 37/177/228, wherein the amino acid positions are relative to the reference sequence corresponding to residues 13 to 468 of SEQ ID NO: 442, or relative to the reference sequence corresponding to SEQ ID NO: 442.

In some embodiments, the amino acid sequence of the engineered RNase inhibitor comprises at least a substitution or substitution set 78T/260Y, 396A, 345S, 37C, 342H, 395V, 367V, 395L, 367L, 38A, 107V, 228K, 260L, 57V, 50M/51H, 338V, 285R/291E/385A, 175F, 60K, 228Q, 145S/147P/148Q, 395M, 60S, 177V, 367A, 260S, 28A, 118R, 64V, 37A/60K/142Q/176S/228L/291S/317S, 60K/64R/317S/319G/342A, 661/228L/319G/342M/395L, 37A/142Q/291S/317S, 64R/107D/142Q/291S/319G/342A/395L, 37A/60K/317S/319G/342A/395L, 37A/60K/107D/142Q/176S/317S/319G/342M, 142Q/317S/319G/342M/395L, 176S/228L/319G/395L, 228L/291S/342M/395L, 64R/395L, 64R/107D/142Q/176S/228L/319G/342M/395L, 395E, 60K/64R/107D/142Q/291S/319G/342M, 37A/66I/107D/291S/317S, 37A/66I/107D/317S/395L, 37A/228L/317S/395L, 342A/395L, 661/142Q/319G/342M/395L, 661/142Q/228L/395L, 363M, 142Q/395L, 60K/64R/177T/291S/395L, 661/319G, 64R/107D/177T/228L/291S/317S/395L, 342M/395L, 64R/142Q/177T/228L/317S, 37C, 260L, 60K/228L/317S/395L, 60K/64R/176S/317S/342M, 177T/228L/342M/395L, 64R/142Q/177T/317S/319G, 60K/64R/107D/342M, 60K/107D/142Q/291S/317S/342M/395L, 66I/228L/319G, 64R/228L/317S/319G, 168F, 319G/395L, 37A/107D/228L, 661/142Q, 37A/342M, 60K/64R/176S/228L/395L, 60K/64R/342M, 35R, 60K/661/395L, 319G, 291S/317S/319G/342M/395L, 37A/177T, 107V, 37A, 37A/60K/64R/142Q/291S, 37A/60K/142Q/228L/291S, 60K/319G/342A, 661/176S/317S/319G/395L, or 37A/177T/228L, wherein the amino acid positions are relative to the reference sequence corresponding to residues 13 to 468 of SEQ ID NO: 442, or relative to the reference sequence corresponding to SEQ ID NO: 442.

In some embodiments, the amino acid sequence of the engineered RNase inhibitor comprises at least a substitution or substitution set A78T/H260Y, S396A, T345S, S37C, S342H, A395V, Q367V, A395L, Q367L, E38A, T107V, R228K, H260L, S57V, S50M/R51H, Q338V, Q285R/P291E/G385A, A175F, Q60K, R228Q, V145S/E147P/N148Q, A395M, Q60S, R177V, Q367A, H260S, T28A, S118R, S64V, S37A/Q60K/S142Q/K176S/R228L/P291S/E317S, Q60K/S64R/E317S/A319G/S342A, T66I/R228L/A319G/S342M/A395L, S37A/S142Q/P291S/E317S,

S64R/T107D/S142Q/P291S/A319G/S342A/A395L, S37A/Q60K/E317S/A319G/S342A/A395L, S37A/Q60K/T107D/S142Q/K176S/E317S/A319G/S342M, S142Q/E317S/A319G/S342M/A395L, K176S/R228L/A319G/A395L, R228L/P291S/S342M/A395L, S64R/A395L, S64R/T107D/S142Q/K176S/R228L/A319G/S342M/A395L, A395E, Q60K/S64R/T107D/S142Q/P291S/A319G/S342M, S37A/T66I/T107D/P291S/E317S, S37A/T66I/T107D/E317S/A395L, S37A/R228L/E317S/A395L, S342A/A395L, T66I/S142Q/A319G/S342M/A395L, T66I/S142Q/R228L/A395L, C363M, S142Q/A395L, Q60K/S64R/R177T/P291S/A395L, T66I/A319G, S64R/T107D/R177T/R228L/P291S/E317S/A395L, S342M/A395L, S64R/S142Q/R177T/R228L/E317S, S37C, H260L, Q60K/R228L/E317S/A395L, Q60K/S64R/K176S/E317S/S342M, R177T/R228L/S342M/A395L, S64R/S142Q/R177T/E317S/A319G, Q60K/S64R/T107D/S342M, Q60K/T107D/S142Q/P291S/E317S/S342M/A395L, T66I/R228L/A319G, S64R/R228L/E317S/A319G, S168F, A319G/A395L, S37A/T107D/R228L, T66I/S142Q, S37A/S342M, Q60K/S64R/K176S/R228L/A395L, Q60K/S64R/S342M, Q35R, Q60K/T66I/A395L, A319G, P291S/E317S/A319G/S342M/A395L, S37A/R177T, T107V, S37A, S37A/Q60K/S64R/S142Q/P291S, S37A/Q60K/S142Q/R228L/P291S, Q60K/A319G/S342A, T66I/K176S/E317S/A319G/A395L, or S37A/R177T/R228L, wherein the amino acid positions are relative to the reference sequence corresponding to residues 13 to 468 of SEQ ID NO: 442, or relative to the reference sequence corresponding to SEQ ID NO: 442.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 578, or to the reference sequence corresponding to SEQ ID NO: 578, wherein the amino acid sequence comprises one or more substitutions relative to the reference sequence corresponding to residues 13 to 468 of SEQ ID NO: 578, or relative to the reference sequence corresponding to SEQ ID NO: 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 726-824, or to the reference sequence corresponding to an even-numbered SEQ ID NO. of SEQ ID NOs: 726-824, wherein the amino acid sequence comprises one or more substitutions relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least a substitution set at amino acid positions 28/118/175/260/338/367, 118/168/260/396, 38/60/168/260/338/367, 28/38/57/60/260/363/367, 57/118/260/338/367, 57/118/168/177/260/338/367, 260/396, 28/118/168/260/367/396, 28/260/338, 28/60/168/260, 57/60/118/175/177/260/338, 28/57/260/338/367, 35/260/338/367, 118/175/260/338, 168/260, 57/60/168/177/260/338/367, 28/168/260/338, 260/367, 28/57/137/260, 38/60/175/260/338, 38/60/260/367/396, 57/60/260/363/367, 60/260/367, 28/38/60/260/367, 60/168,260/363/367, 177/260/363/367, 38/260, 28/60/118/177/260, 28/35/60/118/175/260, 60/367, 28/60/118, 57/118,118/260/338/396, 28/38/57/60/118/177/260/338/363/367/396, 28/38/60/260/363/367, 28/35/38/57/118/260/363/367, 28/57/60/168/260/367, 168/175/260/338/367, 57/60/168/260/338/363/367/396, 60/168/363/367/396, 28/38/57/60/118/168/260/367, 28/175/260/367, 60/363/367/396, 260/338/367, 28/57/260/338/363/367, 168/260/367, 367/396, 28/35/175/260/367/396, or 57/60/168/260/367, wherein the amino acid positions are relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least a substitution set 28A/118R/175F/260S/338V/367L, 118R/168F/260Y/396A, 38A/60S/168F/260L/338V/367L, 28A/38A/57V/60S/260L/363M/367L, 57V/118R/260L/338V/367L, 57V/118R/168F/177V/260L/338V/367L, 260Y/396A, 28A/118R/168F/260L/367L/396A, 28A/260L/338V, 28A/60K/168I/260L, 57V/60K/118R/175F/177V/260L/338V, 28A/57V/260Y/338V/367L, 35R/260S/338V/367L, 118R/175F/260L/338V, 168F/260L, 57V/60S/168F/177V/260S/338V/367L, 28A/168F/260S/338V, 260Y/367L, 28A/57V/137V/260L, 38A/60K/175F/260L/338V, 38A/60K/260S/367A/396A, 57V/60K/260S/363M/367V, 60K/260L/367A, 28A/38A/60K/260S/367L, 60S/168F, 260L/363M/367A, 177V/260Y/363M/367V, 38A/260L, 28A/60K/118R/177V/260Y, 28A/35R/60S/118R/175F/260Y, 60S/367L, 28A/60S/118R, 57V/118R, 118R/260L/338V/396A, 28A/118R/168F/260L/367L/396A, 28A/38A/57V/60K/118R/177V/260Y/338V/363M/367L/396A, 28A/38A/60S/260L/363M/367L, 28A/35R/38A/57V/118R/260L/363M/367L, 28A/57V/60S/168F/260L/367A, 168F/175F/260L/338V/367L, 57V/60S/168F/260Y/338V/363M/367V/396A, 60S/168F/363M/367L/396A, 28A/38A/57V/60K/118R/168F/260L/367L, 28A/175F/260L/367L, 28A/38A/57V/60S/260L/363M/367L, 60S/363M/367L/396A, 260S/338V/367L, 28A/57V/260Y/338V/363M/367L, 168F/260L/367L, 367L/396A, 28A/35R/175F/260Y/367L/396A, or 57V/60K/168F/260L/367L, wherein the amino acid positions are relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least a substitution set T28A/S118R/A175F/H260S/Q338V/Q367L, S118R/S168F/H260Y/S396A,

E38A/Q60S/S168F/H260L/Q338V/Q367L, T28A/E38A/S57V/Q60S/H260L/C363M/Q367L, S57V/S118R/H260L/Q338V/Q367L, S57V/S118R/S168F/R177V/H260L/Q338V/Q367L, H260Y/S396A, T28A/S118R/S168F/H260L/Q367L/S396A, T28A/H260L/Q338V, T28A/Q60K/S168I/H260L, S57V/Q60K/S118R/A175F/R177V/H260L/Q338V, T28A/S57V/H260Y/Q338V/Q367L, Q35R/H260S/Q338V/Q367L, S118R/A175F/H260L/Q338V, S168F/H260L, S57V/Q60S/S168F/R177V/H260S/Q338V/Q367L, T28A/S168F/H260S/Q338V, H260Y/Q367L, T28A/S57V/L137V/H260L, E38A/Q60K/A175F/H260L/Q338V, E38A/Q60K/H260S/Q367A/S396A, S57V/Q60K/H260S/C363M/Q367V, Q60K/H260L/Q367A, T28A/E38A/Q60K/H260S/Q367L, Q60S/S168F, H260L/C363M/Q367A, R177V/H260Y/C363M/Q367V, E38A/H260L, T28A/Q60K/S118R/R177V/H260Y, T28A/Q35R/Q60S/S118R/A175F/H260Y, Q60S/Q367L, T28A/Q60S/S118R, S57V/S118R, S118R/H260L/Q338V/S396A, T28A/S118R/S168F/H260L/Q367L/S396A,

T28A/E38A/S57V/Q60K/S118R/R177V/H260Y/Q338V/C363M/Q367L/S396A, T28A/E38A/Q60S/H260L/C363M/Q367L, T28A/Q35R/E38A/S57V/S118R/H260L/C363M/Q367L, T28A/S57V/Q60S/S168F/H260L/Q367A, S168F/A175F/H260L/Q338V/Q367L, S57V/Q60S/S168F/H260Y/Q338V/C363M/Q367V/S396A, Q60S/S168F/C363M/Q367L/S396A, T28A/E38A/S57V/Q60K/S118R/S168F/H260L/Q367L, T28A/A175F/H260L/Q367L, T28A/E38A/S57V/Q60S/H260L/C363M/Q367L, Q60S/C363M/Q367L/S396A, H260S/Q338V/Q367L, T28A/S57V/H260Y/Q338V/C363M/Q367L, S168F/H260L/Q367L, Q367L/S396A,

T28A/Q35R/A175F/H260Y/Q367L/S396A, or S57V/Q60K/S168F/H260L/Q367L, wherein the amino acid positions are relative to the reference sequence corresponding to residues 13 to 468 of SEQ ID NO: 578, or relative to the reference sequence corresponding to SEQ ID NO: 578.

In some embodiments, the amino acid sequence of the engineered RNase inhibitor comprises at least a substitution at an amino acid position provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least one substitution provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or substitution set at the amino acid position(s) set forth in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the amino acid sequence of the engineered RNase inhibitor comprises at least a substitution or substitution set of an RNase inhibitor variant provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 13 to 468 of an engineered RNase inhibitor set forth in Tables 8.1, 9.1, 9.2, 10.1, 11.1, 12.1, 12.2, 13.1, 13.2, 14.1, and 14.2, or to the sequence of an engineered RNase inhibitor set forth in Tables 8.1, 9.1, 9.2, 10.1, 11.1, 12.1, 12.2, 13.1, 13.2, 14.1, and 14.2.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 13 to 468 of SEQ ID NO: 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 768, 770, 772, 774, 776, 778, 780, 782, 784, 786, 788, 790, 792, 794, 796, 798, 800, 802, 804, 806, 808, 810, 812, 814, 816, 818, 820, 822, or 824.

In some embodiments, the engineered RNase inhibitor comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, or 824.

In some embodiments, the engineered RNase inhibitor comprises an amino acid sequence comprising residues 13 to 468 of an even numbered SEQ ID NO. of SEQ ID NOs: 26-824, or an amino acid sequence comprising an even numbered SEQ ID NO. of SEQ ID NOs: 26-824. In some embodiments, the amino acid sequence of the engineered RNase inhibitor optionally includes 1, 2, 3, 4, 5, 6, 7, 8, 9, or up to 10 substitutions, insertions, and/or deletions. In some embodiments, the amino acid sequence of the engineered RNase inhibitor optionally includes 1, 2, 3, 4, 5, 6, 7, 8, 9, or up to 10 substitutions. In some embodiments, the amino acid sequence of the engineered RNase inhibitor optionally includes 1, 2, 3, 4, or 5 substitutions, insertions, and/or deletions. In some embodiments, the amino acid sequence of the engineered RNase inhibitor optionally includes 1, 2, 3, 4, or 5 substitutions.

In some embodiments, the engineered RNase inhibitor comprises an amino acid sequence comprising residues 13 to 468 of SEQ ID NO: 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, or 824. In some embodiments, the amino acid sequence of the engineered RNase inhibitor optionally includes 1, 2, 3, 4, 5, 6, 7, 8, 9, or up to 10 substitutions, insertions, and/or deletions. In some embodiments, the amino acid sequence of the engineered RNase inhibitor optionally includes 1, 2, 3, 4, 5, 6, 7, 8, 9, or up to 10 substitutions. In some embodiments, the amino acid sequence of the engineered RNase inhibitor optionally includes 1, 2, 3, 4, or 5 substitutions, insertions, and/or deletions. In some embodiments, the amino acid sequence of the engineered RNase inhibitor optionally includes 1, 2, 3, 4, or 5 substitutions.

In some embodiments, the engineered RNase inhibitor comprises an amino acid sequence comprising SEQ ID NO: 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, or 824. In some embodiments, the amino acid sequence of the engineered RNase inhibitor optionally includes 1, 2, 3, 4, 5, 6, 7, 8, 9, or up to 10 substitutions, insertions, and/or deletions. In some embodiments, the amino acid sequence of the engineered RNase inhibitor optionally includes 1, 2, 3, 4, 5, 6, 7, 8, 9, or up to 10 substitutions. In some embodiments, the amino acid sequence of the engineered RNase inhibitor optionally includes 1, 2, 3, 4, or 5 substitutions, insertions, and/or deletions. In some embodiments, the amino acid sequence of the engineered RNase inhibitor optionally includes 1, 2, 3, 4, or 5 substitutions.

In some embodiments, the engineered RNase inhibitor comprises an amino acid sequence comprising residues 13 to 468 of SEQ ID NO: 36, 238, 320, 360, 442, or 578, or an amino acid sequence comprising SEQ ID NO: 36, 238, 320, 360, 442, or 578. In some embodiments, the amino acid sequence of the engineered RNase inhibitor optionally includes 1, 2, 3, 4, 5, 6, 7, 8, 9, or up to 10 substitutions, insertions, and/or deletions. In some embodiments, the amino acid sequence of the engineered RNase inhibitor optionally includes 1, 2, 3, 4, 5, 6, 7, 8, 9, or up to 10 substitutions. In some embodiments, the amino acid sequence of the engineered RNase inhibitor optionally includes 1, 2, 3, 4, or 5 substitutions, insertions, and/or deletions. In some embodiments, the amino acid sequence of the engineered RNase inhibitor optionally includes 1, 2, 3, 4, or 5 substitutions.

In some of the foregoing embodiments, the engineered RNase inhibitor polypeptide has 1, 2, 3, 4, or up to 5 substitutions in the amino acid sequence. In some embodiments, the engineered RNase inhibitor polypeptide has 1, 2, 3, or 4 substitutions in the amino acid sequence. In some embodiments, the substitutions comprises non-conservative or conservative substitutions. In some embodiments, the substitutions comprises conservative substitutions. In some embodiments, the substitutions comprises non-conservative substitutions. In some embodiments, guidance on non-conservative and conservative substitutions are provided by the variants disclosed herein.

In some embodiments, the engineered RNase inhibitor of the present disclosure has RNase inhibitory activity, particularly with one or more of an improved or enhanced property described herein. In some embodiments, the engineered RNase inhibitor has at least one improved or enhanced properties as compared to a reference RNase inhibitor.

In some embodiments, the engineered RNase inhibitor displays increased RNase inhibitory activity, particularly inhibitory activity against RNase A, as compared to a reference RNase inhibitor. In some embodiments, the engineered RNase inhibitor displays at least 1.05 fold, 1.1 fold, 1.15 fold, 1.2 fold, 1.3 fold, 1.4 fold, 1.5 fold, 1.6 fold, 1.8 fold, 2 fold, 3 fold, 4 fold, 5 fold, 6 fold, 7 fold, 8 fold, 9 fold, 10 fold, or more increase in inhibitory activity as compared to the reference RNase inhibitor.

In some embodiments, the engineered RNase inhibitor displays increased stability as compared to a reference RNasse inhibitor. In some embodiments, the engineered RNase inhibitor displays increased pH stability as compared to a reference RNase inhibitor.

In some embodiments, the engineered RNase inhibitor displays increased thermostability as compared to a reference RNasse inhibitor. In some embodiments, the engineered RNase inhibitor shows increased thermostabiltiy at temperature 40° C. or greater, 45° C. or greater, 50° C. or greater, 55° C. or greater, 60° C. or greater, 65° C. or greater, or 70° C. or greater, up to 75° C., as compared to a reference RNase inhibitor. In some embodiments, the engineered RNase inhibitor displays increased thermostability at a temperature range of 50° C. to 75° C. as compared to a reference RNase inhibitor.

In some embodiments, the engineered RNase inhibitor displays increased resistance to oxidation compared to a reference RNase inhibitor. In some embodiments, the resistance to oxidation is in presence of hydrogen peroxide. In some embodiments, the engineered RNase inhibitor maintains activity in the absence of a reducing agent, such as DTT.

In some embodiments, the engineered RNase inhibitor displays increased expression as soluble protein as compared to a reference RNase inhibitor.

In some embodiments, the reference RNase inhibitor has the sequence corresponding to residues 13 to 468 of SEQ ID NO: 2, 36, 238, 320, 360, 442, or 578, or the sequence corresponding to SEQ ID NO: 2, 36, 238, 320, 360, 442, or 578. In some embodiments, the reference RNase inhibitor has the sequence corresponding to residues 13 to 468 of SEQ ID NO: 2, or the sequence corresponding to SEQ ID NO: 2.

In some embodiments, the engineered RNase inhibitor has one or more improved property selected from i) increased inhibitory activity against RNase A, ii) increased stability, iii) increased thermostability, iv) increased resistance to oxidation, and v) increased expression as soluble protein, or any combination of i), ii), iii), iv) and v), as compared to a reference RNase inhibitor. In some embodiments, the reference RNase inhibitor has the sequence corresponding to residues 13 to 468 of SEQ ID NO: 2, 36, 238, 320, 360, 442, or 578, or the sequence corresponding to SEQ ID NO: 2, 36, 238, 320, 360, 442, or 578. In some embodiments, the reference RNase inhibitor has the sequence corresponding to residues 13 to 468 of SEQ ID NO: 2, or the sequence corresponding to SEQ ID NO: 2.

In some embodiments, the present disclosure further provides an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to
(a) residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) SEQ ID NO: 2, 4, 6, 8, 10, 12, 14, 16, 18, 20, 22, or 24.

In some embodiments, the engineered RNase inhibitor comprises an amino acid sequence comprising:
(a) residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) SEQ ID NO: 2, 4, 6, 8, 10, 12, 14, 16, 18, 20, 22, or 24.

In some embodiments, the engineered RNase inhibitor is expressed as a fusion protein. In some embodiments, the engineered RNase inhibitor described herein can be fused to a variety of polypeptide sequences, such as, by way of example and not limitation, polypeptide tags that can be used for detection and/or purification. In some embodiments, the fusion protein of the engineered RNase inhibitor comprises a glycine-histidine or histidine-tag (His-tag). In some embodiments, the fusion protein of the engineered RNase inhibitor comprises an epitope tag, such as c-myc, FLAG, V5, or hemagglutinin (HA). In some embodiments, the fusion protein of the engineered RNase inhibitor comprises a GST, SUMO, Strep, MBP, or GFP tag. In some embodiments, the fusion is to the amino (N—) terminus of engineered RNase inhibitor polypeptide. In some embodiments, the fusion is to the carboxy (C—) terminus of the engineered RNase inhibitor polypeptide.

In some embodiments, the engineered RNase inhibitor polypeptide described herein is an isolated composition. In some embodiments, the engineered RNase inhibitor polypeptide is purified, as further discussed herein.

In some embodiments, the present disclosure further provides functional fragments or biologically active fragments of engineered RNase inhibitor polypeptides described herein. Thus, for each and every embodiment herein of an engineered RNase inhibitor, a functional fragment or biologically active fragment of the engineered RNase inhibitor is provided herewith. In some embodiments, a functional fragment or biologically active fragments of an engineered RNase inhibitor comprises at least about 90%, at least about 95%, at least about 96%, at least about 97%, at least about 98%, or at least about 99% of the activity of the RNase inhibitor polypeptide from which it was derived (i.e., the parent RNase inhibitor). In some embodiments, functional fragments or biologically active fragments comprise at least 90%, 91%, 92%, 93%, 94%, 95%, 96%, 97%, 98%, or 99% of the parent sequence of the RNase inhibitor. In some embodiments, the functional fragment will be truncated by less than 5, less than 10, less than 15, less than 10, less than 25, less than 30, less than 35, less than 40, less than 45, less than 50 amino acids, less than 55 amino acids, less than 60 amino acids, less than 65 amino acids, or less than 70 amino acids.

In some embodiments, a functional fragment of an engineered RNase inhibitor herein comprises at least 70%, 75%, 80%, 81%, 82%, 83%, 84%, 85%, 90%, 91%, 92%, 93%, 94%, 95%, 96%, 97%, 98%, or 99% of the parent sequence of the engineered RNase inhibitor. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered RNase inhibitor polypeptide described herein include at least a mutation or mutation set in the amino acid sequence of the engineered RNase inhibitor described herein. Accordingly, in some embodiments, the functional fragments or biologically active fragments of the engineered RNase inhibitor displays the enhanced or improved property associated with the mutation or mutation set in the parent RNase inhibitor.

### Polynucleotides Encoding Engineered Polypeptides, Expression Vectors and Host Cells

In another aspect, the present disclosure provides recombinant polynucleotides encoding the engineered RNase inhibitor described herein. In some embodiments, the recombinant polynucleotides are operably linked to one or more heterologous regulatory sequences that control gene expression to create a recombinant polynucleotide construct capable of expressing the engineered RNase inhibitor. In some embodiments, an expression construct containing at least one heterologous polynucleotide encoding the engineered RNase inhibitor polypeptide(s) is introduced into appropriate host cells to express the corresponding RNase inhibitor polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered RNase inhibitor of the present disclosure. Thus, the present disclosure provides methods and compositions for the production of each and every possible variation of polynucleotides that could be made that encode the engineered RNase inhibitor polypeptides described herein by selecting combinations based on the possible codon choices, and all such polynucleotide sequence variations are to be considered specifically disclosed for any polypeptide described herein, including the amino acid sequences presented in the Examples (e.g., in Tables 8.1, 9.1, 9.2, 10.1, 11.1, 12.1, 12.2, 13.1, 13.2, 14.1, 14.2, and the Appendix) and in the Sequence Listing.

In some embodiments, the codons are preferably optimized for utilization by the chosen host cell for protein production. In some embodiments, preferred codons in bacteria are used for expression in bacteria. In some embodiments, preferred codons in fungal cells are used for expression in fungal cells. In some embodiments, preferred codons in insect cells are used for expression in insect cells. In some embodiments, preferred codons in mammalian cells are used for expression in mammalian cells. In some embodiments, codon optimized polynucleotides encoding an engineered RNase inhibitor polypeptide described herein contain preferred codons at about 40%, 50%, 60%, 70%, 80%, 90%, or greater than 90% of the codon positions in the full-length coding region.

Accordingly, in some embodiments, a recombinant polynucleotide of the present disclosure comprises a polynucleotide sequence encoding an engineered RNase inhibitor polypeptides described herein. In some embodiments, the polynucleotide sequence of the recombinant polynucleotide is codon optimized.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of an even numbered SEQ ID NO. of SEQ ID NOs: 2 and 26-824, or to a reference sequence corresponding to an even numbered SEQ ID NO. of SEQ ID NOs: 2 and 26-824, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of SEQ ID NO: 2, 36, 238, 320, 360, 442, or 578, or to a reference sequence corresponding to SEQ ID NO: 2, 36, 238, 320, 360, 442, or 578, wherein the amino acid sequence comprises one or more substitutions relative to the reference sequence corresponding to residues 13 to 468 of SEQ ID NO: 2, 36, 238, 320, 360, 442, or 578, or to the reference sequence corresponding to SEQ ID NO: 2, 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 2, or to the reference sequence corresponding to SEQ ID NO: 2, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-142, or to a reference sequence corresponding to an even numbered SEQ ID NO. of SEQ ID NOs: 26-142, wherein the amino acid sequence comprises one or more substitutions relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence comprising at least a substitution at amino acid position 2, 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence comprising at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence comprising at least a mutation at position 95, 296, or 459, or combinations thereof, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence comprising at least a substitution or substitution set at amino acid position(s) 257/459, 345, 257, 296, 113, 95/296/459, 95/257/296, 453, 107/267, 417/422/459, 395, 390, 257/296, 243, 156/257, 351, 348, 363, 95/257/296/422, 83/107/230/267/429, 107/429, 107/323/429, 94/121/265/390, 400, 429, or 81, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an RNase inhibitor comprising an amino acid sequence comprising at least a substitution at an amino acid position provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an RNase inhibitor comprising an amino acid sequence comprising at least one substitution provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an RNase inhibitor comprising an amino acid sequence comprising at least a substitution or substitution set at the amino acid position(s) provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an RNase inhibitor comprising an amino acid sequence comprising at least a substitution or substitution set of an RNase inhibitor variant provided in Tables 8.1, 9.1, 9.2, 10.1, 11.1, 12.1, 12.2, 13.1, 13.2, 14.1, 14.2, and the Appendix, wherein the amino acid positions are relative to the reference sequence corresponding to residues 13 to 468 of SEQ ID NO: 2, or relative to the reference sequence corresponding to SEQ ID NO: 2.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, 238, 320, 360, 442, or 578, or to the reference sequence corresponding to SEQ ID NO: 36, 238, 320, 360, 442, or 578, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least a substitution at amino acid position 50, 64, 81, 90, 95, 107, 113, 124, 142, 145, 147, 148, 176, 203, 205, 228, 243, 257, 272, 285, 291, 296, 319, 323, 342, 348, 390, 395, 452, 453, or 459, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least a substitution at amino acid position 95, 296, or 459, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 36, or to the reference sequence corresponding to SEQ ID NO: 36, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 144-316, or to the reference sequence corresponding to an even-numbered SEQ ID NO. of SEQ ID NOs: 144-316, wherein the amino acid sequence comprises one or more substitutions relative to the reference sequence corresponding to residues 13 to 468 of SEQ ID NO: 36, or relative to the reference sequence corresponding to SEQ ID NO: 36.

In some embodiments, the recombinant polynucleotide comprises a polynucleotide sequence encoding an engineered RNase inhibitor comprising an amino acid sequence comprising at least a substitution or substitution set at amino acid position(s) 323/390/429, 257/323/429, 257/390, 257/323/390, 257/323/390/429, 257/429, 257/377/390, 135/257/323/377/390, 323/377/390/429, 257/323/377/390, 257/377/429, 158/257/323/377/390, 257/323/377/429, 167/257/323/390/429, 377/390/429, 377/390, 257/323/377/390/429, 257/377, 390/429, 257/377/390/429, 257/390/429, 390, 243/363/390, 113/257/351/363/429, 113/257/351/390/430, 113/257/272/390, 113/243/351/390/429, 257/272/345/348, 243/257/351/390, 113/243/257/272/390, 257/272/363/390/430, 257/351/390, 113/243/390/430, 113/257/267/351/363/400/430, 113/243/257/351/430, 243/267/341/390/395, 113/243/257/345/348, 113/243/257/348/390, 243/345/348/390, 113/243/257/345/430, 257/345/348/351/390/429/430, 272/345/390/429/430, 243/267/351, 243/272/345/390, 113/257/363/390, 243/390/429, 113/243/257/351/390, 113/243/272/345/348/430, 243/267/272/348/390, 113/257/272/345/348/363/430, 113/243/390/429/430, 113/243/257/272/351/363, 243/272/345/348/363/390/395, 113/243/272/351/363/390/395, 113/243/257/272/345/390/430, 113/243/267/272/363/390/430, 113/243/267/348/363/430, 113/243/257/351/390/430, 113/243/257/272/351/390, 113/243/267/390/430, 243/257/348/395/430, 243/345/348/429/430, 243/257/345/348/395, 243/257/272/351/400/429, 113/243/267, 113/390/430, 243/267/351/400, or 429/430, wherein the amino acid positions are relative to the reference sequence corresponding to residues 13 to 468 of SEQ ID NO: 36, or relative to the reference sequence corresponding to SEQ ID NO: 36.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 238, or to the reference sequence corresponding to SEQ ID NO: 238, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 318-352, or to the reference sequence corresponding to an even-numbered SEQ ID NO. of SEQ ID NOs: 318-352, wherein the amino acid sequence comprises one or more substitutions relative to the reference sequence corresponding to residues 13 to 468 of SEQ ID NO: 238, or relative to the reference sequence corresponding to SEQ ID NO: 238.

In some embodiments, the recombinant polynucleotide comprises a polynucleotide sequence encoding an engineered RNase inhibitor comprising an amino acid sequence comprising at least a substitution or substitution set at amino acid position(s) 323/453, 323/348/452, 452/453, 323/351/430/452/453, 291, 145, 228, 323/429, 38, 150, 12, 173, 38/237, or 40, wherein the amino acid positions are relative to the reference sequence corresponding to residues 13 to 468 of SEQ ID NO: 238, or relative to the reference sequence corresponding to SEQ ID NO: 238.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 320, or to the reference sequence corresponding to SEQ ID NO: 320, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 354-390, or to the reference sequence corresponding to an even-numbered SEQ ID NO. of SEQ ID NOs: 354-390, wherein the amino acid sequence comprises one or more substitutions relative to the reference sequence corresponding to residues 13 to 468 of SEQ ID NO: 320, or relative to the reference sequence corresponding to SEQ ID NO: 320.

In some embodiments, the recombinant polynucleotide comprises a polynucleotide sequence encoding an engineered RNase inhibitor comprising an amino acid sequence comprising at least a substitution or substitution set at amino acid position(s) 13/145/228/291/348/351, 453, 12/13/453, 145/228/291/453, 13/145/453, 145/291/453, 38/145/453, 12/145/453, 38/145/228/453, 15/28/81/124/221/285/317, 15/28/64/203/221/285/317, 15/57/81/124/148/203, 12/453, 56/57/64/81/91/124/203/285/317, 66/90/142/176/205/319, 228/453, or 145/228/453, wherein the amino acid positions are relative to the reference sequence corresponding to residues 13 to 468 of SEQ ID NO: 320, or relative to the reference sequence corresponding to SEQ ID NO: 320.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 360, or to the reference sequence corresponding to SEQ ID NO: 360, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 392-554, or to the reference sequence corresponding to an even-numbered SEQ ID NO. of SEQ ID NOs: 392-554, wherein the amino acid sequence comprises one or more substitutions relative to the reference sequence corresponding to residues 13 to 468 of SEQ ID NO: 360, or relative to the reference sequence corresponding to SEQ ID NO: 360.

In some embodiments, the recombinant polynucleotide comprises a polynucleotide sequence encoding an engineered RNase inhibitor comprising an amino acid sequence comprising at least a substitution or substitution set at amino acid position(s) 319, 37, 60, 342, 81, 50, 395, 64, 177, 107, 373, 205, 35, 78, 228, 178, 285, 37/142/148/203/205/285/319, 50/81/90/124/147/148/203/205/285, 66/90/148/285/317/319, 28/37/91/142/205/285, 50/91/124/142/147/148/203/205/317/319, 50/66/90/91/124/142/285/317, 147/148/203/285/317/319, 50/66/81/90/205/319, 50/66/124/203/205/317/319, 28/66/84/90/91/142/205/319, 37/91/148/205/319, 28/66/81/124/147/285/319, 28/50/66/84/90/147/203/205/317, 28/50/90/91/124/142/319, 37/81/147/148/203/205, 28/66/81/90/91/124/203/285/317/319, 28/50/66/81/90/91/138/147/148/205/285/319, 28/37/50/124/319, 81/90/91/124/285/317, 50/90/142/203/285, 50/90/124/142/205/285/319, 28/66/90/91/147/148/205/319/326, 66/81/319, 28/66/90/203/205/319, 50/66/90/91/205, 50/66/147/205, 37/81/285, 50/66/90/91/319, 50/147/203/285, 37/50/81/148, 28/66/142/319, 15/28/66/81/90/91/147/148/319, 15/28/81/90/91/142/285/317/319, 15/28/37/50/66/124/147/148/205/285, 15/66/81/147/148, 66/91/124/142/147/148, 15/37/66/81/147/148/317/319, 15/50/90/91/124/142/147/285/317/319, 15/50/90/91/142/147/148/203/205, 15/28/81/142/147/148/285/317/319, 15/28/50/66/90/91/203/285/319, 15/66/90/91/147/148/205/285, 15/28/90/91/142/147/148/285/317/319, 15/37/91/148/285/319, 81/147/148/203/205, 147/148/285/319, 15/50/90/147/148, 15/66/81/90/285/319, 50/81/147/148/285, 37/147/148/285, 15/37/66/84/90/91/203/285/317, 15/84/90/91/203/285, 37/90/91/147, 90/91/205/317/319, 50/148/203/317, 15/28/50/66/81/84/90/285/317, 15/81/147, or 15/285, wherein the amino acid positions are relative to the reference sequence corresponding to residues 13 to 468 of SEQ ID NO: 360, or relative to the reference sequence corresponding to SEQ ID NO: 360.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 442, or to the reference sequence corresponding to SEQ ID NO: 442, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 556-724, or to the reference sequence corresponding to an even-numbered SEQ ID NO. of SEQ ID NOs: 556-724, wherein the amino acid sequence comprises one or more substitutions relative to the reference sequence corresponding to residues 13 to 468 of SEQ ID NO: 442, or relative to the reference sequence corresponding to SEQ ID NO: 442.

In some embodiments, the recombinant polynucleotide comprises a polynucleotide sequence encoding an engineered RNase inhibitor comprising an amino acid sequence comprising at least a substitution or substitution set at amino acid position(s) 78/260,396, 345, 37, 342, 395, 367, 38, 107, 228, 260, 57, 50/51, 338, 285/291/385, 175, 60, 145/147/148, 177, 28, 118, 64, 37/60/142/176/228/291/317, 60/64/317/319/342, 66/228/319/342/395, 37/142/291/317, 64/107/142/291/319/342/395, 37/60/317/319/342/395, 37/60/107/142/176/317/319/342, 142/317/319/342/395, 176/228/319/395, 228/291/342/395, 64/395, 64/107/142/176/228/319/342/395, 60/64/107/142/291/319/342, 37/66/107/291/317, 37/66/107/317/395, 37/228/317/395, 342/395, 66/142/319/342/395, 66/142/228/395, 363, 142/395, 60/64/177/291/395, 66/319, 64/107/177/228/291/317/395, 64/142/177/228/317, 60/228/317/395, 60/64/176/317/342, 177/228/342/395, 64/142/177/317/319, 60/64/107/342, 60/107/142/291/317/342/395, 66/228/319, 64/228/317/319, 168, 319/395, 37/107/228, 66/142, 37/342, 60/64/176/228/395, 60/64/342, 35, 60/66/395, 319, 291/317/319/342/395, 37/177,107, 37/60/64/142/291, 37/60/142/228/291, 60/319/342, 66/176/317/319/395, or 37/177/228, wherein the amino acid positions are relative to the reference sequence corresponding to residues 13 to 468 of SEQ ID NO: 442, or relative to the reference sequence corresponding to SEQ ID NO: 442.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of SEQ ID NO: 578, or to the reference sequence corresponding to SEQ ID NO: 578, wherein the amino acid sequence comprises one or more substitutions relative to the reference sequence corresponding to residues 13 to 468 of SEQ ID NO: 578, or relative to the reference sequence corresponding to SEQ ID NO: 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 13 to 468 of an even-numbered SEQ ID NO. of SEQ ID NOs: 726-824, or to the reference sequence corresponding to an even-numbered SEQ ID NO. of SEQ ID NOs: 726-824, wherein the amino acid sequence comprises one or more substitutions relative to the reference sequence corresponding to residues 13 to 468 of SEQ ID NO: 578, or relative to the reference sequence corresponding to SEQ ID NO: 578.

In some embodiments, the recombinant polynucleotide comprises a polynucleotide sequence encoding an engineered RNase inhibitor comprising an amino acid sequence comprising at least a substitution set at amino acid position(s) 28/118/175/260/338/367, 118/168/260/396, 38/60/168/260/338/367, 28/38/57/60/260/363/367, 57/118/260/338/367, 57/118/168/177/260/338/367, 260/396, 28/118/168/260/367/396, 28/260/338, 28/60/168/260, 57/60/118/175/177/260/338, 28/57/260/338/367, 35/260/338/367, 118/175/260/338, 168/260, 57/60/168/177/260/338/367, 28/168/260/338, 260/367, 28/57/137/260, 38/60/175/260/338, 38/60/260/367/396, 57/60/260/363/367, 60/260/367, 28/38/60/260/367, 60/168,260/363/367, 177/260/363/367, 38/260, 28/60/118/177/260, 28/35/60/118/175/260, 60/367, 28/60/118, 57/118,118/260/338/396, 28/38/57/60/118/177/260/338/363/367/396, 28/38/60/260/363/367, 28/35/38/57/118/260/363/367, 28/57/60/168/260/367, 168/175/260/338/367, 57/60/168/260/338/363/367/396, 60/168/363/367/396, 28/38/57/60/118/168/260/367, 28/175/260/367, 60/363/367/396, 260/338/367, 28/57/260/338/363/367, 168/260/367, 367/396, 28/35/175/260/367/396, or 57/60/168/260/367, wherein the amino acid positions are relative to the reference sequence corresponding to residues 13 to 468 of SEQ ID NO: 578, or relative to the reference sequence corresponding to SEQ ID NO: 578.

In some embodiments, the recombinant polynucleotide comprises a polynucleotide sequence encoding an engineered RNase inhibitor comprising an amino acid sequence comprising at least a substitution at an amino acid position provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least one substitution provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least a substitution or substitution set at the amino acid position(s) of an RNase inhibitor variant provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least a substitution or substitution set of an RNase inhibitor variant provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence comprising at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 8.1, 9.1, 9.2, 10.1, 11.1, 12.1, 12.2, 13.1, 13.2, 14.1, and 14.2, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 13 to 468 of an engineered RNase inhibitor set forth in Tables 8.1, 9.1, 9.2, 10.1, 11.1, 12.1, 12.2, 13.1, 13.2, 14.1, and 14.2, or to the sequence of an engineered RNase inhibitor set forth in Tables 8.1, 9.1, 9.2, 10.1, 11.1, 12.1, 12.2, 13.1, 13.2, 14.1, and 14.2.

In some embodiments, the recombinant polynucleotide comprises a polynucleotide sequence encoding an engineered RNase inhibitor comprising an amino acid sequence comprising residues 13 to 468 of an even numbered SEQ ID NO. of SEQ ID NOs: 26-824, or an amino acid sequence comprising an even numbered SEQ ID NO. of SEQ ID NOs: 26-824, optionally wherein the amino acid sequence has 1, 2, 3, 4, 5, 6, 7, 8, 9, or up to 10 substitutions.

In some embodiments, the recombinant polynucleotide comprises a polynucleotide sequence encoding the engineered RNase inhibitor comprising an amino acid sequence comprising residues 13 to 468 of SEQ ID NO: 36, 238, 320, 360, 442, or 578, or an amino acid sequence comprising SEQ ID NO: 36, 238, 320, 360, 442, or 578, optionally wherein the amino acid sequence has 1, 2, 3, 4, 5, 6, 7, 8, 9, or up to 10 substitutions in the amino acid sequence.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 37 to 1404 of SEQ ID NO: 35, 237, 319, 359, 441, or 577, or to a reference polynucleotide sequence corresponding to SEQ ID NO: 35, 237, 319, 359, 441, or 577, wherein the recombinant polynucleotide encodes an RNase inhibitor.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 37 to 1404 of an odd numbered SEQ ID NO. of SEQ ID NOs: 25-823, or to a reference polynucleotide sequence corresponding to an odd numbered SEQ ID NO. of SEQ ID NOs: 25-823, wherein the recombinant polynucleotide encodes an RNase inhibitor.

In some embodiments, the recombinant polynucleotide comprises a polynucleotide sequence having at least 70%, 75%, 80%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 37 to 1404 of SEQ ID NO: 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, or 823, wherein the recombinant polynucleotide encodes an RNase inhibitor.

In some embodiments, the recombinant polynucleotide comprises a polynucleotide sequence having at least 70%, 75%, 80%, 85%, 86%, 87%, 88%, 89%, 90%, 91%, 92%, 93%, 94%, 95%, 96%, 97%, 98%, 99% or more sequence identity to the reference polynucleotide sequence corresponding to SEQ ID NO: 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, or 823, wherein the recombinant polynucleotide encodes an RNase inhibitor.

In some embodiments, the recombinant polynucleotide comprises a polynucleotide sequence comprising nucleotide residues 37 to 1404 of SEQ ID NO: 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, or 823.

In some embodiments, the recombinant polynucleotide comprises a polynucleotide sequence comprising SEQ ID NO: 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, or 823.

In some embodiments, the recombinant polynucleotide comprises a polynucleotide sequence comprising nucleotide residues 37 to 1404 of SEQ ID NO. 35, 237, 319, 359, 441, or 577, or a polynucleotide sequence comprising SEQ ID NOs: 35, 237, 319, 359, 441, or 577.

In some embodiments, the present disclosure provides a recombinant polynucleotide capable of hybridizing under highly stringent conditions to a reference polynucleotide encoding an engineered RNase inhibitor polypeptide described herein, e.g., a recombinant polynucleotide provided in Tables 8.1, 9.1, 9.2, 10.1, 11.1, 12.1, 12.2, 13.1, 13.2, 14.1, and 14.2, or a reverse complement thereof. In some embodiments, the recombinant polynucleotide hybridizes under highly stringent conditions to a reference polynucleotide corresponding to nucleotide residues 37 to 1404 of SEQ ID NO. 35, 237, 319, 359, 441, or 577, or a polynucleotide sequence comprising SEQ ID NOs: 35, 237, 319, 359, 441, or 577, or a reverse complement thereof. In some embodiments, the recombinant polynucleotide hybridizes under highly stringent conditions to a polynucleotide corresponding to nucleotide residues 37 to 1404 of an odd numbered SEQ ID NO. of SEQ ID NOs: 25-823, or a polynucleotide corresponding to an odd numbered SEQ ID NO. of SEQ ID NOs: 25-823, or a reverse complement thereof.

In some embodiments, the present disclosure provides a recombinant polynucleotide capable of hybridizing under highly stringent conditions to a reverse complement of a reference polynucleotide encoding an engineered RNase inhibitor polypeptide described herein, wherein the recombinant polynucleotide hybridizing under stringent conditions encodes an RNase inhibitor polypeptide comprising an amino acid sequence having one or more amino acid differences as compared to SEQ ID NO: 2, 36, 238, 320, 360, 442, or 578, at residue positions selected from any positions as set forth in Tables 8.1, 9.1, 9.2, 10.1, 11.1, 12.1, 12.2, 13.1, 13.2, 14.1, and 14.2. In some embodiments, the recombinant polynucleotide that hybridizes under highly stringent conditions to a reverse complement of a reference polynucleotide encoding an engineered RNase inhibitor polypeptide described herein encodes an RNase inhibitor polypeptide having one or more amino acid differences present in an engineered RNase inhibitor having an amino acid sequence corresponding to residues 13 to 468 of an even numbered SEQ ID NO. of SEQ ID NOs: 24-582, or an amino acid sequence comprising an even numbered SEQ ID NO. of SEQ ID NOs: 24-582, wherein the amino acid differences are relative to SEQ ID NO: 14, 42, or 204.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 37 to 1404 of SEQ ID NO: 35, 237, 319, 359, 441, or 577, or to a reference polynucleotide sequence corresponding to SEQ ID NO: 35, 237, 319, 359, 441, or 577, or a reverse complement thereof. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 37 to 1404 of an odd-numbered SEQ ID NO. of SEQ ID NOs: 25-823, or an odd-numbered SEQ ID NO. of SEQ ID NOs: 25-823, or a reverse complement thereof.

In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 37 to 1404 of SEQ ID NO: 35, 237, 319, 359, 441, or 577, or to a reference polynucleotide sequence corresponding to SEQ ID NO: 35, 237, 319, 359, 441, or 577 encodes an engineered RNase inhibitor polypeptide. In some embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 37 to 1404 of an odd-numbered SEQ ID NO. of SEQ ID NOs: 25-823, or an odd-numbered SEQ ID NO. of SEQ ID NOs: 25-823 encodes an engineered RNase inhibitor polypeptide.

In some embodiments, the recombinant polynucleotide comprises a polynucleotide sequence encoding an engineered RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to
(a) residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) SEQ ID NO: 2, 4, 6, 8, 10, 12, 14, 16, 18, 20, 22, or 24.

In some embodiments, the recombinant polynucleotide comprises a polynucleotide sequence encoding an engineered RNase inhibitor comprising an amino acid sequence comprising
(a) residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) SEQ ID NO: 2, 4, 6, 8, 10, 12, 14, 16, 18, 20, 22, or 24.

In some embodiments, the recombinant polynucleotide comprises a polynucleotide sequence comprising at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to
(a) nucleotide residues 34 to 1401 of SEQ ID NO: 3; nucleotide residues 34 to 1401 of SEQ ID NO: 5; nucleotide residues 34 to 1416 of SEQ ID NO: 7; nucleotide residues 34 to 1401 of SEQ ID NO: 9; nucleotide residues 34 to 1416 of SEQ ID NO: 11; nucleotide residues 34 to 1401 of SEQ ID NO: 13; nucleotide residues 34 to 1401 of SEQ ID NO: 15; nucleotide residues 34 to 1413 of SEQ ID NO: 17; nucleotide residues 34 to 1401 of SEQ ID NO: 19; nucleotide residues 34 to 1401 of SEQ ID NO: 21; or nucleotide residues 34 to 1401 of SEQ ID NO: 23; or
(b) SEQ ID NO: 1, 3, 5, 7, 9, 11, 13, 15, 17, 19, 21, or 23, wherein the recombinant polynucleotide encodes an RNase inhibitor.

In some embodiments, the recombinant polynucleotide comprises a polynucleotide sequence comprising
(a) nucleotide residues 34 to 1401 of SEQ ID NO: 3; nucleotide residues 34 to 1401 of SEQ ID NO: 5; nucleotide residues 34 to 1416 of SEQ ID NO: 7; nucleotide residues 34 to 1401 of SEQ ID NO: 9; nucleotide residues 34 to 1416 of SEQ ID NO: 11; nucleotide residues 34 to 1401 of SEQ ID NO: 13; nucleotide residues 34 to 1401 of SEQ ID NO: 15; nucleotide residues 34 to 1413 of SEQ ID NO: 17; nucleotide residues 34 to 1401 of SEQ ID NO: 19; nucleotide residues 34 to 1401 of SEQ ID NO: 21; or nucleotide residues 34 to 1401 of SEQ ID NO: 23; or
(b) SEQ ID NO: 1, 3, 5, 7, 9, 11, 13, 15, 17, 19, 21, or 23.

In some embodiments, a recombinant polynucleotide encoding any of the RNase inhibitors herein is manipulated in a variety of ways to facilitate expression of the RNase inhibitor polypeptide. In some embodiments, the recombinant polynucleotide encoding the RNase inhibitor comprises expression vectors where one or more control sequences, particularly heterologous control sequences, is present to regulate the expression of the RNase inhibitor polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators.

In some embodiments, suitable promoters are selected based on the host cells selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in insect cells include, but are not limited to, polyhedrin, p10, ELT, OpIE2, and hr5/iel promoters. Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-la or glyceraldehyde-3-phosphate dehydrogenase, and from Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the RNase inhibitor polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412 (5): 793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., supra). Exemplary terminators for mammalian cells include, but are not limited to those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, a non-translated region of an mRNA that is important for translation by the host cell. In some embodiments, the leader sequence is operably linked to the 5′ terminus of the nucleic acid sequence encoding the RNase inhibitor polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence is a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (see, e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for insect and mammalian host cells include but are not limited to, those from the genes for immunoglobulin gamma (IgG) and the signal peptide in a human secreted protein, such as human beta-galactosidase polypeptide.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present disclosure provides an expression vector comprising a recombinant polynucleotide encoding an engineered RNase inhibitor polypeptide, and one or more expression regulating regions such as a promoter and a terminator, a replication origin, etc., depending on the type of hosts into which they are to be introduced. In some embodiments, the various nucleic acid and control sequences described herein are joined together (i.e., operably linked) to produce recombinant expression vectors which include one or more convenient restriction sites to allow for insertion or substitution of the nucleic acid sequence encoding the RNase inhibitor polypeptide at such sites. Alternatively, in some embodiments, the nucleic acid sequence of the present disclosure is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the encoded RNase inhibitor polypeptide. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising a recombinant polynucleotide encoding at least one engineered RNase inhibitor polypeptide described herein, the polynucleotide(s) being operably linked to one or more control sequences for expression of the engineered RNase inhibitor polypeptide(s) in the host cell. In some embodiments, the host cell comprises an expression vector comprising a recombinant polynucleotide encoding an engineered RNase inhibitor polypeptide described herein, where the polynucleotide is operably linked to one or more control sequences. Host cells suitable for use in expressing the polypeptides encoded by the expression vectors of the present disclosure are known in the art and include but are not limited to, bacterial cells, such as E. coli, B. subtilis, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (AfhuA) and BL21).

In another aspect, the present disclosure provides a method of producing the engineered RNase inhibitor polypeptides, where the method comprises culturing a host cell capable of expressing a polynucleotide encoding the engineered RNase inhibitor polypeptide under conditions suitable for expression of the polypeptide such that the engineered RNase inhibitor is produced. In some embodiments, the method further comprises the step(s) of isolating the RNase inhibitor polypeptides from the culture and/or host cells. In some embodiments, the method further comprises purifying the expressed RNase inhibitor polypeptide, as described herein.

Appropriate culture media and growth conditions for host cells are known in the art. It is contemplated that any suitable method for introducing polynucleotides for expression of the RNase inhibitor polypeptides into cells will find use in the present invention. Suitable techniques include, but are not limited to electroporation, biolistic particle bombardment, liposome mediated transfection, calcium chloride transfection, and protoplast fusion.

In some embodiments, recombinant polypeptides (e.g., RNase inhibitor polypeptide variants) can be produced using any suitable methods known the art. For example, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (region-specific), or random mutagenesis over the entire gene (e.g., saturation mutagenesis). Numerous suitable methods are known to those in the art to generate polypeptide variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased inhibitory activity, or low or reduced activity, increased thermal activity, increased stability, increased substrate range, increased inhibitor resistance or tolerance, increased salt tolerance, and/or pH stability, etc.).

In some embodiments, the engineered RNase inhibitor polypeptides with the properties disclosed herein can be obtained by subjecting the polynucleotide encoding the naturally-occurring or engineered RNase inhibitor polypeptide to a suitable mutagenesis and/or directed evolution methods known in the art, for example, as described herein. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3: S136-S140), and cassette mutagenesis (see, e.g., Black et al., Proc. Natl. Acad. Sci. USA, 1996, 93:3525-3529).

Mutagenesis and directed evolution methods can be applied to RNase inhibitor-encoding polynucleotides to generate variant libraries that can be expressed, screened, and assayed. Any suitable mutagenesis and directed evolution methods find use in the present disclosure and are known in the art (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254 (2): 157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the clones obtained following mutagenesis treatment are screened by subjecting the polypeptide preparations to a defined treatment conditions or assay conditions (e.g., buffer, temperature, pH condition, RNA substrate, etc.) and measuring polypeptide activity after the treatments or other suitable assay conditions. Clones containing a polynucleotide encoding the polypeptide of interest are then isolated from the gene, sequenced to identify the nucleotide sequence changes (if any), and used to express the polypeptide in a host cell. Measuring polypeptide activity from the expression libraries can be performed using any suitable method known in the art and as described in the Examples.

For engineered polypeptides of known sequence, the polynucleotides encoding the polypeptide can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence (see, e.g., Hughes et al., Cold Spring Harb Perspect Biol. 2017 January; 9 (1): a023812). For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer), purified, annealed, ligated and cloned in appropriate vectors.

In some embodiments, a method for preparing the engineered RNase inhibitor polypeptide can comprise: (a) synthesizing a polynucleotide encoding a polypeptide comprising an amino acid sequence of any RNase inhibitor as described herein, and (b) expressing the RNase inhibitor polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

In some embodiments, any of the engineered RNase inhibitor polypeptides expressed in a host cell are recovered and/or purified from the cells and/or the culture medium using any one or more of the known techniques for protein purification, including, among others, lysozyme treatment, sonication, filtration, salting-out, ultra-centrifugation, and chromatography.

Chromatographic techniques for isolation and purification of the RNase inhibitor polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular polypeptide may depend, in part, on factors such as net charge, hydrophobicity, hydrophilicity, molecular weight, molecular shape, etc., and will be apparent to those having skill in the art. In some embodiments, affinity techniques may be used to isolate the improved RNase inhibitor polypeptides. For affinity chromatography purification, any antibody that specifically binds an RNase inhibitor polypeptide of interest can be used. For the production of antibodies, various host animals, including but not limited to rabbits, mice, rats, etc., are immunized by injection with an RNase inhibitor polypeptide, or a fragment thereof. In some embodiments, the RNase inhibitor polypeptide or fragment is attached to a suitable carrier, such as BSA, by means of a side chain functional group or linkers attached to a side chain functional group. Where the engineered RNase inhibitor includes a fusion polypeptide that allows for affinity purification, such as a His-tag, standard affinity methods for the particular fusion protein can be used.

### Composition of RNase Inhibitors

In a further aspect, the present disclosure provides compositions of the RNase inhibitors disclosed herein. In some embodiments, the composition comprises at least one engineered RNase inhibitor polypeptide described herein. In some embodiments, the engineered RNase inhibitor polypeptide in the composition is isolated or purified. In some embodiments, the RNase inhibitor is combined with other components and compounds to provide compositions and formulations comprising the engineered RNase inhibitor polypeptide as appropriate for different applications and uses.

In some embodiments, the composition comprises at least one engineered RNase inhibitor described herein. For example, a composition comprises at least one engineered RNase inhibitor provided in Tables 8.1, 9.1, 9.2, 10.1, 11.1, 12.1, 12.2, 13.1, 13.2, 14.1, and 14.2.

In some embodiments, the composition comprises an RNase inhibitor provided in Table 3.3. In some embodiments, the composition comprises an RNase inhibitor with an amino acid sequence comprising: (a) residues 13 to 468 of SEQ ID NO: 2; residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) SEQ ID NO: 2, 4, 6, 8, 10, 12, 14, 16, 18, 20, 22, or 24.

In some embodiments, the composition further comprises one or more of a buffer and an RNA substrate. In some embodiments, the buffer includes a reducing agent, such as dithiothreitol. In some embodiments, the composition further comprises a cryoprotecting agent, including, among others, glycerol, polyethylene glycol, Ficoll, or dextran. In some embodiments, dextran can also act as an inhibitor of RNase in the composition. In some embodiments, the composition further comprises a molecular crowding agent, including, among others, bovine serum albumin (BSA), polyethylene glycol, dextran, and/or Ficoll. In some embodiments, the composition comprising an engineered RNase inhibitor is a lyophilizate.

In some embodiments, the composition comprises an engineered RNase inhibitor disclosed herein, and a nucleic acid modifying enzyme. In some embodiments, the nucleic acid modifying enzyme comprises a polymerase. In some embodiments, the polymerase comprises an RNA polymerase. In some embodiments, the polymerase comprises a reverse transcriptase. In some embodiments, the polymerase comprises a DNA dependent DNA polymerase, in particular a thermal DNA polymerase. In some embodiments, the polymerase comprises a terminal transferase or poly-A polymerase, or other template-independent polymerases. In some embodiments, the nucleic acid modifying enzyme comprises an RNA ligase. In some embodiments, the nucleic acid modifying enzyme comprises a polynucleotide kinase or phosphatase.

In some embodiments, an engineered RNase inhibitor described herein is provided in solution, as a lyophilizate, or immobilized on a substrate. In some embodiments, the RNase inhibitor is provided on a substrate, such as a solid substrate, porous substrate, membrane, or particles. The polypeptide can be entrapped in matrixes or membranes. In some embodiments, matrices include polymeric materials such as calcium-alginate, agar, k-carrageenin, polyacrylamide, and collagen, or solid matrices, such as activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the RNase inhibitor is immobilized on the surface of a support material. In some embodiments, the polypeptide is adsorbed on the support material. In some embodiments, the polypeptide is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

### Uses of Engineered RNase Inhibitor Polypeptides and Kits

In another aspect, the present disclosure provides uses of the engineered RNase inhibitors in applications involving RNA and/or where RNase may be present. In some embodiments, the engineered RNase inhibitor is used to inhibit RNase activity, the method comprising contacting an RNase with an engineered RNase inhibitor described herein under conditions suitable for inhibiting RNase activity.

In some embodiments, the suitable conditions comprises a temperature of about 4° C. to about 75° C. In some embodiments, the suitable conditions comprises a temperature of about 25° C. to about 75° C. In some embodiments, the suitable conditions comprises a temperature of about 50° C. to about 75° C. In some embodiments, the suitable conditions comprises a temperature of about 4° C. to about 50° C. In some embodiments, the suitable conditions comprises a temperature of about 4° C. to about 25° C.

In some embodiments, the engineered RNase inhibitor is used when isolating RNA. In some embodiments, the engineered RNase inhibitor is added to a sample containing or suspected of containing RNA. In some embodiments, the sample is a biological sample, such as cells, tissues, including biopsy and autopsy samples, frozen sections taken for histological purposes, blood, plasma, serum, sputum, stool, tears, mucus, hair, skin, etc. In some embodiments, the biological sample are cells or viruses, such as from a bacterial culture, virus culture, or cell culture. In some embodiments, the sample is an environmental sample, including, among others, water, including samples from ocean, river, refuse/sewer, etc., soil, air, vents, or surfaces, such as floors, machinery, counters, etc.

In some embodiments, the engineered RNase inhibitor is used in combination with a reverse transcriptase in preparing a cDNA copy of an RNA template, such as for preparation of cDNA libraries or diagnostics for a target RNA, such as bacterial, fungal or viral RNA.

In some embodiments, the engineered RNase inhibitor is used for in vitro transcription reactions, for example with an RNA polymerase. By way of example and not limitation, the in vitro transcription can use a T7 RNA polymerase or engineered T7 RNA polymerases or other bacterial or viral RNA polymerases, and a DNA template of interest. In some embodiments, the in vitro transcription can be a coupled in vitro transcription and translation system for in vitro synthesis of proteins.

In some embodiments, the engineered RNase inhibitor is used in RT-PCR, RT-qPCR, and RT-LAMP reactions. In particular, the increased thermostability of the engineered RNase inhibitor makes it suitable in RT-PCR applications.

In some embodiments, the engineered RNase inhibitor is used in RNA microarray or RNA sequencing applications, such as for transcriptome-wide analysis of differential gene expression and differential splicing of mRNAs (see, e.g., Stark et al., Nature Reviews Genetics, 2019, 20:631-656).

In some embodiments, the engineered RNase inhibitor is used in combination with an RNA ligase in ligating RNA, such as reactions using RNA ligase 1 and/or RNA ligase 2 to ligate RNA fragments. In some embodiments, the engineered RNase inhibitor is used in reactions for ligation of modified RNA fragments, where the modifications include, among others, 2′-O-alkyl, 2′-halo, and/or phosphorothioate internucleotide linkages.

In some embodiments, the engineered RNase inhibitor is used to enhance CRISPR mediated engineering of genomes in cells (see, e.g., Laoharawee et al., Int J Mol Sci., 2022, 23 (17): 9749). Introduction on the RNase inhibitor into the cell subject to CRISPR mediated engineering can enhance CRISPR mediated genome editing.

In a further aspect, the present disclosure provides a kit comprising an RNase inhibitor or a composition thereof described herein. In some embodiments, the kit further comprises at least a buffer. In some embodiments, the buffer includes a reducing agent, e.g., dithiothreitol. In some embodiments, the composition further comprises a cryoprotecting agent, such as glycerol, polyethylene glycol (e.g., PEG 6000 and PEG 8000), or dextran. In some embodiments, the composition further comprises a molecular crowding agent, including, among others, bovine serum albumin (BSA), polyethylene glycol, dextran, and/or Ficoll. In some embodiments, the composition comprises an RNA substrate. In some embodiments, the RNase inhibitor is provided as a lyophilizate.

In some embodiments, the kit further comprises a nucleic acid modifying enzyme other than the RNase inhibitor. In some embodiments, the nucleic acid modifying enzyme comprises a polymerase. In some embodiments, the polymerase comprises an RNA polymerase. In some embodiments, the polymerase comprises a reverse transcriptase. In some embodiments, the polymerase comprises a DNA dependent DNA polymerase, in particular a thermal DNA polymerase. In some embodiments, the polymerase comprises a terminal transferase or poly-A polymerase, or other template-independent polymerase acting on an RNA substrate. In some embodiments, the nucleic acid modifying enzyme comprises an RNA ligase, a polynucleotide kinase, or a phosphatase.

### EXAMPLES

The following Examples, including experiments and results achieved, are provided for illustrative purposes only and are not to be construed as limiting the present invention.

In the experimental disclosure below, the following abbreviations where relevant apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); ul, uL, μl, and μL (microliter); cm (centimeters); mm (millimeters); um and μm (micrometers); sec. (seconds); min(s) (minute(s)); h(s) and hr(s) (hour(s)); U (units); OD (optical density); MW (molecular weight); rpm (rotations per minute); rcf (relative centrifugal force); psi and PSI (pounds per square inch); ° C. (degrees Celsius); RT and rt (room temperature); ds (double stranded); ss (single stranded); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FPLC (fast protein liquid chromatography); ddH2O (double distilled water); PBS (phosphate buffered saline); BSA (bovine serum albumin); DTT (dithiothreitol); CAM (chloramphenicol); CAT (chloramphenicol acetyltransferase); IPTG (isopropyl β-D-1-thiogalactopyranoside); FIOPC or FIOP (fold improvements over positive control or parent); LB (Luria-Bertani); TB (Terrific-Broth).

### Example 1

E. coli Expression Hosts Containing Recombinant Ribonuclease Inhibitor (RNH1) Genes

The initial ribonuclease inhibitor (RNase inhibitor or RI) used to produce the variants of the present disclosure was SEQ ID NO: 2 cloned into the expression vector pCK110900 (See, FIG. 3 of US Pat. Appln. Publn. No. 2006/0195947) operatively linked to the lac promoter under control of the lacl repressor. The expression vector also contains the P15a origin of replication and the chloramphenicol resistance gene. E. coli W3110 were transformed with the resulting plasmids, using standard methods known in the art. The transformants were isolated by subjecting the cells to chloramphenicol selection, as known in the art (See e.g., U.S. Pat. No. 8,383,346 and WO2010/144103).

### Example 2

### Preparation of Shake-Flask Purified RNase Inhibitor

### Shake-Flask Expression

Selected cultures grown as described above were plated onto Luria Broth (LB) agar plates with 1% glucose and 30 μg/ml chloramphenicol and grown overnight at 37° C. A single colony from each culture was transferred to 5 ml of LB broth with 1% glucose and 30 μg/ml chloramphenicol. The cultures were grown for 20 h at 30° C., 250 rpm, and sub-cultured at a dilution of approximately 1:50 into 250 ml of Terrific Broth with 30 μg/ml of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4000 rpm×10 min. The culture supernatant liquid was discarded, and the pellets were resuspended in 30 mL of 50 mM Tris-HCl, pH 8.0. This cell suspension was chilled in an ice bath and lysed using a Microfluidizer cell disruptor (Microfluidics M-110L). The crude lysate was pelleted by centrifugation (10,000 rpm for 60 min at 4° C.), and the supernatant liquid was then filtered through a 0.2 μm PES membrane to further clarify the lysate.

Purification of RNase Inhibitor from Shake-Flask Lysates

RNase inhibitor lysates were purified using an AKTA Pure purification system and a 5-mL HisTrap FF column (GE Healthcare); the run parameters are provided in Table 2.1. The shake-flask wash buffer comprised 50 mM Tris-HCl pH 8.0, 500 mM NaCl, 20 mM imidazole, 0.02% v/v Triton X-100 reagent, the elution buffer comprised 50 mM Tris-HCl pH 8.0, 500 mM NaCl, 250 mM imidazole, 0.02% v/v Triton X-100 reagent, and the storage buffer comprised 40 mM Tris-HCl pH 8.0, 100 mM KCl, 0.1 mM EDTA, and 50% (v/v) glycerol.

TABLE 2.1
Purification Parameters

| Parameter | Volume | |
|---|---|---|
| Column volume | 5 | ml |
| Pressure limit | 0.3 | MPa |
| Sample volume | 40 | mL |
| Wash Unbound volume | 20 CV = 100 mL | |
| Elution | Isocratic (step) | |
| Elution volume | 5 CV = 25 mL | |
| Fraction volume | 1.5 | mL |

| | |
|---|---|
| Re-equilibration volume | 5 CV = 25 mL |

The three or four most concentrated 1.5 mL fractions based on UV absorption (A280) were pooled and a fraction thereof was dialyzed overnight in storage buffer in a 3.5K Slide-A-Lyzer™ dialysis cassette (ThermoFisher) for buffer exchange. RNase inhibitor concentrations in the preparations were measured by absorption at 280 nm, normalized to 2 mg mL-1 with storage buffer and stored at −20° C. until further use.

### Example 3

### Gene Acquisition-Activity of RNase Inhibitor Homologs

Genes encoding RNase inhibitors, as provided in Table 3.1, were used to prepare RNA inhibitor polypeptides for initial screening. The sequence identity between each of the RNase inhibitors is shown in Table 3.2. A solution of RNase A was prepared by diluting RNase A (ThermoFisher, cat #EN0531) to a concentration of 10 μg mL-1 in isothermal buffer (New England Biolabs, cat #B0537S, 20 mM Tris-HCl, 10 mM (NH₄)₂SO₄, 50 mM KCl, 2 mM MgSO₄, 0.1% Tween® 20, pH 8.8). Diluted RNase A solution (10 μL) was added to each well of a skirted 384-well PCR plate. Purified RNase inhibitor (as prepared in Example 2, 10 μL) was added to each well of the skirted 384-well PCR plate containing RNase A. The plate was briefly vortexed and centrifuged to mix, then incubated at 37° C. for 15 minutes. RNaseAlert substrate was prepared by resuspending in 1 mL TE buffer and mixing 1:1 with 10× RNaseAlert buffer. Following incubation, the RNaseAlert assay was initiated by adding the mixture of purified RNase inhibitor and RNase A solution (8 μL per well) to a skirted 384-well PCR plate containing RNaseAlert substrate and buffer mixture (2 μL per well). The plate was briefly vortexed and centrifuged to mix, then inserted into a CFX Touch 384-well Real-Time PCR Detection System (Bio-Rad). The plate was incubated at 37° C. and increase of fluorescence from RNaseAlert substrate cleavage was monitored in the FAM channel. Wells with less RNase inhibition result in a higher RNaseAlert substrate cleavage and thus higher relative fluorescence units (RFU). RNase inhibitor activity was calculated as the ratio of RFU of the no inhibitor negative control to the last data point collected for a given sample and is shown in Table 3.3.

TABLE 3.1
RNH1 genes assayed, with source
organism and UniProt ID for each.

| SEQ ID NO: | Source organism | UniProtKB_ID |
|---|---|---|
| 1/2 | Ailuropoda melanoleuca | GILVD8 |
| 3/4 | Odobenus rosmarus divergens | A0A2U3ZJ48 |
| 5/6 | Heterocephalus glaber | G5C479 |
| 7/8 | Erinaceus europaeus | A0A1S3WTP7 |
| 9/10 | Mus musculus | Q91VI7 |
| 11/12 | Loxodonta africana | G3SPV7 |
| 13/14 | Pteropus alecto | L5KQY7 |
| 15/16 | Desmodus rotundus | K9IKE3 |
| 17/18 | Callithrix jacchus | F7HNE6 |
| 19/20 | Trichechus manatus latirostris | A0A2Y9E8W7 |
| 21/22 | Canis lupus familiaris | F1PK29 |
| 23/24 | Alligator sinensis | A0A1U7RRY1 |

TABLE 3.2

| | SEQ ID | SEQ ID | SEQ ID | SEQ ID | SEQ ID | SEQ ID | SEO ID | SEQ ID | SEQ ID | SEQ ID | SEO ID | SEQ ID |
|---|---|---|---|---|---|---|---|---|---|---|---|---|
| | NO: 2 | NO: 4 | NO: 6 | NO: 8 | NO: 10 | NO: 12 | NO: 14 | NO: 16 | NO: 18 | NO: 20 | NO: 22 | NO: 24 |
| SEQ ID | 100 | 88.9 | 68.3 | 63.5 | 74.9 | 70.3 | 74.3 | 68.5 | 67.6 | 67.4 | 79.6 | 51.5 |
| NO: 2 | | | | | | | | | | | | |
| SEQ ID | 88.9 | 100 | 68.7 | 64.6 | 73.8 | 69.8 | 72.7 | 67.8 | 66 | 68.4 | 81.2 | 51.4 |
| NO: 4 | | | | | | | | | | | | |
| SEQ ID | 68.9 | 68.7 | 100 | 61.2 | 70.6 | 68.1 | 67.8 | 65.9 | 63.6 | 65.9 | 65.5 | 48.8 |
| NO: 6 | | | | | | | | | | | | |
| SEQ ID | 65.5 | 64.6 | 61.2 | 100 | 67.1 | 64.5 | 64.3 | 62.7 | 60.6 | 65.2 | 61.4 | 48.5 |
| NO: 8 | | | | | | | | | | | | |
| SEQ ID | 74.9 | 73.8 | 70.6 | 67.1 | 100 | 69.4 | 70.6 | 66.3 | 68.1 | 68 | 69.9 | 51 |
| NO: 10 | | | | | | | | | | | | |
| SEQ ID | 70.3 | 69.8 | 68.1 | 64.5 | 69.4 | 100 | 68.6 | 64.6 | 643 | 85.2 | 63.9 | 50 |
| NO: 12 | | | | | | | | | | | | |
| SEQ ID | 74.3 | 72.7 | 67.8 | 64.3 | 70.6 | 68.6 | 100 | 72.5 | 65.6 | 68.2 | 69.1 | 48.4 |
| NO: 14 | | | | | | | | | | | | |
| SEQ ID | 68.5 | 67.8 | 633 | 62.7 | 66.3 | 64.6 | 72.5 | 108 | 63.5 | 64.8 | 64 | 48.8 |
| NO: 16 | | | | | | | | | | | | |
| SEQ ID | 67.6 | 66 | 65.6 | 60.6 | 68.1 | 64.3 | 65.6 | 63.5 | 100 | 64.3 | 63.7 | 48.9 |
| NO: 18 | | | | | | | | | | | | |
| SEQ ID | 67.4 | 68.4 | 65.9 | 65.2 | 68 | 85.2 | 68.2 | 64.8 | 64.3 | 100 | 61.6 | 49 |
| NO: 20 | | | | | | | | | | | | |
| SEQ ID | 79.6 | 81.2 | 63.5 | 61.4 | 69.9 | 63.9 | 69.1 | 64 | 63.7 | 61.8 | 100 | 48.6 |
| NO: 22 | | | | | | | | | | | | |
| SEQ ID | 51.5 | 51.4 | 48.8 | 48.3 | 51 | 50 | 48.4 | 48.8 | 48.9 | 49 | 48.6 | 100 |
| NO: 24 | | | | | | | | | | | | |

TABLE 3.3
RNase Inhibitor Activity of Variants
Relative to No Inhibitor Control

| SEQ ID NO: | Activity Ratio |
|---|---|
| (nt/aa) | (Relative to No Inhibitor Control) |
| 1/2 | +++ |
| 3/4 | +++ |
| 5/6 | ++ |
| 7/8 | ++ |
| 9/10 | ++ |
| 11/12 | ++ |
| 13/14 | + |
| 15/16 | + |
| 17/18 | + |
| 19/20 | + |
| 21/22 | + |
| 23/24 | + |

¹ Levels of increased activity were determined relative to the No Inhibitor Control and defined as follows: “+” 1.03 to 1.40-fold increased activity; “++” > 1.40-fold increased activity; “+++” > 1.70-fold increased activity.

### Example 4

### Preparation of HTP Ribonuclease Inhibitor (RNH1)-Containing Wet Cell Pellets

E. coli cells containing recombinant RNH1-encoding genes from monoclonal colonies were inoculated into 180 μL LB containing 1% glucose and 30 μg/mL chloramphenicol (CAM) in the wells of 96-well, shallow-well microtiter plates. The plates were sealed with O₂-permeable seals, and cultures were grown overnight at 30° C., 200 rpm, and 85% humidity. Then, 10 μL of each of the cell cultures were transferred into the wells of 96-well, deep-well plates containing 390 mL TB and 30 μg/mL CAM. The deep-well plates were sealed with O₂-permeable seals and incubated at 30° C., 250 rpm, and 85% humidity until OD₆₀₀ 0.6-0.8 was reached. The cell cultures were then induced by IPTG to a final concentration of 1 mM and incubated overnight under the same conditions as originally used. The cells were then pelleted using centrifugation at 4,000 rpm for 10 min. The supernatants were discarded, and the pellets were frozen at −80° C. prior to lysis.

### Example 5

### Preparation of HTP RNH1-Containing Lysozyme-Lysed Cell Lysates

First, 400 μL buffer containing 50 mM Triethanolamine-HCl pH 7.5 were added to the cell paste in each well, produced as described in Example 2. The cells were shaken on a bench-top shaker to resuspend. Resuspended cells (50 μL) were transferred to a 96-well hard-shell PCR plate containing lysozyme in buffer (50 μL of 0.1 g L⁻¹ lysozyme in 50 mM Triethanolamine-HCl pH 7.5) and pipetted to mix. The cell-lysozyme mixture was lysed for 60 minutes in a thermocycler at a set lysis temperature ranging from 48° C. to 56.5° C., specified in the specific example. The plate was then centrifuged for 15 min at 4,000 rpm and 4° C. The clarified supernatant liquids were optionally diluted and used in reactions to determine their ribonuclease inhibitor activity and thermostability.

### Example 6

### Preparation of HTP RNH1-Containing Heat-Treated Cell Lysates

First, 400 μL buffer containing 50 mM Triethanolamine-HCl pH 7.5 were added to the cell paste in each well, produced as described in Example 2. The cells were shaken on a bench-top shaker to resuspend. Resuspended cells (50 μL) were transferred to a 96-well hard-shell PCR plate containing buffer (50 μL of 50 mM Triethanolamine-HCl pH 7.5) and pipetted to mix. The cells were lysed for 60 minutes in a thermocycler at a set lysis temperature ranging from 56° C. to 71° C., specified in the specific example. The plate was then centrifuged for 15 min at 4,000 rpm and 4° C. The clarified supernatant liquids were optionally diluted and used in reactions to determine their ribonuclease inhibitor activity and thermostability.

### Example 7

### Use of Heat-Treated Lysate for Ribonuclease Inhibition Assays

A solution of RNase A was prepared by diluting RNase A (ThermoFisher, cat #EN0531) to a concentration of 1 μg mL-1 in isothermal buffer (New England Biolabs, cat #B0537S; 20 mM Tris-HCl, 10 mM (NH₄)₂SO₄, 50 mM KCl, 2 mM MgSO₄, 0.1% Tween® 20, pH 8.8). Diluted RNase A solution (8 μL) was added to each well of a skirted 384-well PCR plate. RNH1-containing heat-treated cell lysate (as prepared in either Example 5 or Example 6, 8 μL) was added to each well of the skirted 384-well PCR plate containing RNase A. The plate was briefly vortexed and centrifuged to mix, then incubated at 37° C. for 10 minutes. RNaseAlert substrate was prepared by resuspending in 1 mL TE buffer and mixing 1:1 with 10× RNaseAlert buffer. Following incubation, the RNaseAlert assay was initiated by adding the mixture of RNH1-containing heat-treated cell lysate and RNase A solution (4 μL per well) to a skirted 384-well PCR plate containing RNaseAlert substrate and buffer mixture (1 μL per well). The plate was briefly vortexed and centrifuged to mix, then inserted into a CFX Touch 384-well Real-Time PCR Detection System (Bio-Rad). The plate was incubated at 37° C. and increase of fluorescence from RNaseAlert substrate cleavage was monitored in the FAM channel.

### Example 8

Variants with Improvements Over SEQ ID NO: 2

SEQ ID NO: 2 was selected as the parent protein after screening wild-type proteins for both thermostability and ribonuclease inhibitor activity in lysozyme-lysed heat-treated lysates. Libraries of engineered genes were produced using established techniques (e.g., saturation mutagenesis, recombination of previously identified beneficial mutations). The polypeptides encoded by each gene were produced in HTP as described in Example 4, and the soluble lysozyme-lysed cell lysate was generated as described in Example 5, with a lysis temperature of 48° C. The clarified lysate was used to detect ribonuclease inhibitor activity as described in Example 7.

Each sample was evaluated by its residual RNase inhibition activity after heat treatment relative to SEQ ID NO: 2 (activity fold improvement over positive control, FIOP). In this example, activity FIOP is the ratio of SEQ ID NO:2's RFU to the given sample's RFU, both measured after the first qPCR incubation cycle. Activity FIOPs are shown in Table 8.1.

TABLE 8.1
RNase Inhibitor Activity of Variants Relative to SEQ ID NO: 2

| SEQ ID | | Activity FIOP after 48° C. |
|---|---|---|
| NO: | Amino Acid Differences | heat treatment |
| (nt/aa) | (Relative to SEQ ID NO: 2) | (Relative to SEQ ID NO: 2) |
| 25/26 | R257F/G459E | +++ |
| 27/28 | T345A | +++ |
| 29/30 | R257F | +++ |
| 31/32 | M296L | +++ |
| 33/34 | P113G | +++ |
| 35/36 | R95E/M296L/G459E | +++ |
| 37/38 | R257V | +++ |
| 39/40 | R95Q/R257F/M296I | ++ |
| 41/42 | S453M | ++ |
| 43/44 | T107I/V267K | ++ |
| 45/46 | M417L/I422V/G459E | ++ |
| 47/48 | A395L | ++ |
| 49/50 | A390S | ++ |
| 51/52 | R257G/M296I | ++ |
| 53/54 | D243C | ++ |
| 55/56 | V156M/R257S | ++ |
| 57/58 | V351R | ++ |
| 59/60 | T348K | ++ |
| 61/62 | C363E | ++ |
| 63/64 | S453R | ++ |
| 65/66 | R95E/R257F/M296I/I422V | ++ |
| 67/68 | A395C | ++ |
| 69/70 | I94L/T121S/F332L/A390T/V466I | ++ |
| 71/72 | R257G/M417L/E419D/G459E | ++ |
| 73/74 | N452R | ++ |
| 75/76 | R95E/R257F/M296I | ++ |
| 77/78 | A377S/A390T | ++ |
| 79/80 | S453F | + |
| 81/82 | N452K | + |
| 83/84 | D272N | + |
| 85/86 | D243S | + |
| 87/88 | Y126V/D386G | + |
| 89/90 | V267H | + |
| 91/92 | A390G | + |
| 93/94 | E430L | + |
| 95/96 | S453L | + |
| 97/98 | S453A | + |
| 99/100 | V230L/V429L | + |
| 101/102 | A377S/A390S | + |
| 103/104 | R257T | + |
| 105/106 | G459K | + |
| 107/108 | D272Q | + |
| 109/110 | E430G | + |
| 111/112 | S453K | + |
| 113/114 | V83L/T107I/V230L/V267T/V429L | + |
| 115/116 | V267R | + |
| 117/118 | T107I/V429L | + |
| 119/120 | D243R | + |
| 121/122 | D243M | + |
| 123/124 | T107A/Q323E/V429L | + |
| 125/126 | I94L/T121S/I265L/A390T | + |
| 127/128 | V400L | + |
| 129/130 | S453W | + |
| 131/132 | V351K | + |
| 133/134 | V429L | + |
| 135/136 | R257F/M296I | + |
| 137/138 | A395I | + |
| 139/140 | G459M | + |
| 141/142 | H81F | + |

¹ Levels of increased activity were determined relative to the reference polypeptide of SEQ ID NO: 2 and defined as follows: “+” 1.05 to 1.23-fold increased activity; “++” > 1.23-fold increased activity; “+++” > 1.28-fold increased activity.

### Example 9

Variants with Improvements Over SEQ ID NO: 36

SEQ ID NO: 36 was selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble lysozyme-lysed cell lysate was generated as described in Example 5, with a lysis temperature of 52° C. or 56.5° C. The clarified lysate was used to detect ribonuclease inhibitor activity as described in Example 7.

Each sample was evaluated by its residual RNase inhibition activity after heat treatment relative to SEQ ID NO: 36 (activity fold improvement over positive control, FIOP). In this example, activity FIOP is the ratio of SEQ ID NO:36's RFU to the given sample's RFU, both measured after the first qPCR incubation cycle. Activity FIOPs are shown in Table 9.1.

TABLE 9.1
RNase Inhibitor Activity of Variants Relative to SEQ ID NO: 36

| | | Activity FIOP |
|---|---|---|
| | | after 52° C. |
| SEQ ID | Amino Acid Differences | heat treatment |
| NO: | (Relative to SEQ | (Relative to SEQ |
| (nt/aa) | ID NO: 36) | ID NO: 36) |
| 143/144 | Q323E/A390S/V429L | +++ |
| 145/146 | R257F/Q323E/V429L | +++ |
| 147/148 | R257F/A390S | +++ |
| 149/150 | R257F/Q323E/A390T | +++ |
| 151/152 | R257F/Q323E/A390S/V429L | +++ |
| 153/154 | R257F/V429L | +++ |
| 155/156 | R257F/A377T/A390T | +++ |
| 157/158 | A135V/R257F/Q323E/A377S/A390T | +++ |
| 159/160 | Q323E/A377T/A390S/V429L | +++ |
| 161/162 | R257F/Q323E/A390S | +++ |
| 163/164 | R257F/Q323E/A377S/A390S | ++ |
| 165/166 | R257F/Q323E/A377T/A390T | ++ |
| 167/168 | R257F/A377S/V429L | ++ |
| 169/170 | R257F/A390T | ++ |
| 171/172 | Y158C/R257F/Q323E/A377T/A390S | ++ |
| 173/174 | R257F/A377T/V429L | ++ |
| 175/176 | R257F/A377S/A390S | ++ |
| 177/178 | R257F/Q323E/A377S/A390T | ++ |
| 179/180 | R257F/Q323E/A377T/V429L | ++ |
| 181/182 | E167K/R257F/Q323E/A390S/V429L | ++ |
| 183/184 | Q323E/A390T/V429L | ++ |
| 185/186 | R257F/Q323E/A390T/V429L | ++ |
| 187/188 | A377T/A390S/V429L | ++ |
| 189/190 | R257F/A377T/A390S | + |
| 191/192 | A377T/A390S | + |
| 193/194 | R257F/Q323E/A377S/A390S/V429L | + |
| 195/196 | R257F/Q323E/A377S/A390T/V429L | + |
| 197/198 | R257F/Q323E/A377S/V429L | + |
| 199/200 | R257F/A377S | + |
| 201/202 | A390T/V429L | + |
| 203/204 | R257F/Q323E/A377T/A390S | + |
| 205/206 | R257F/Q323E/A377T/A390S/V429L | + |
| 207/208 | R257F/A377T/A390T/V429L | + |
| 209/210 | R257F/A390T/V429L | + |
| 211/212 | R257F/A377T/A390S/V429L | + |
| 213/214 | R257F/A377S/A390T | + |
| 215/216 | R257F/A377S/A390T/V429L | + |
| 217/218 | R257F/A390S/V429L | + |
| 219/220 | A377S/A390T | + |
| 221/222 | A390T | + |

¹ Levels of increased activity were determined relative to the reference polypeptide of SEQ ID NO: 36 and defined as follows: “+” 1.23 to 1.37-fold increased activity; “++” > 1.37-fold increased activity; “+++” > 1.42-fold increased activity.

TABLE 9.2
RNase Inhibitor Activity of Variants Relative to SEQ ID NO: 36

| SEQ ID | | Activity FIOP after 56.5° C. |
|---|---|---|
| NO: | Amino Acid Differences | heat treatment |
| (nt/aa) | (Relative to SEQ ID NO: 36) | (Relative to SEQ ID NO: 36) |
| 223/224 | D243S/C363E/A390G | +++ |
| 225/226 | P113G/R257F/V351K/C363E/V429L | +++ |
| 227/228 | P113G/R257V/V351K/A390S/E430G | +++ |
| 229/230 | P113G/R257V/D272N/A390S | +++ |
| 231/232 | P113G/D243M/V351K/A390S/V429L | +++ |
| 233/234 | R257V/D272N/T345A/T348K | +++ |
| 235/236 | D243S/R257T/V351R/A390S | +++ |
| 237/238 | P113G/D243M/R257T/D272N/A390G | +++ |
| 239/240 | R257T/D272Q/C363E/A390G/E430L | +++ |
| 241/242 | R257V/V351K/A390G | ++ |
| 243/244 | P113G/D243S/A390G/E430G | ++ |
| 245/246 | P113G/R257V/V267H/V351R/C363E/V400L/E430L | ++ |
| 247/248 | P113G/D243M/R257V/V351R/E430G | ++ |
| 249/250 | D243M/V267R/S341C/A390G/A395L | ++ |
| 251/252 | P113G/D243M/R257T/T345A/T348K | ++ |
| 253/254 | P113G/D243S/R257F/T348K/A390G | ++ |
| 255/256 | D243S/T345A/T348K/A390G | ++ |
| 257/258 | P113G/D243M/R257V/T345A/E430L | ++ |
| 259/260 | R257T/T345A/T348K/V351R/A390S/V429L/E430G | ++ |
| 261/262 | D272Q/T345A/A390G/V429L/E430L | ++ |
| 263/264 | D243M/V267R/V351K | ++ |
| 265/266 | D243R/D272N/T345A/A390G | ++ |
| 267/268 | P113G/R257T/C363E/A390G | ++ |
| 269/270 | D243S/A390G/V429L | ++ |
| 271/272 | P113G/D243M/R257F/V351K/A390G | ++ |
| 273/274 | P113G/D243M/D272Q/T345A/T348K/E430G | ++ |
| 275/276 | D243M/V267R/D272Q/T348K/A390G | ++ |
| 277/278 | P113G/R257T/D272Q/T345A/T348K/C363E/E430L | + |
| 279/280 | P113G/D243R/A390G/V429L/E430L | + |
| 281/282 | P113G/D243M/R257F/D272N/V351R/C363E | + |
| 283/284 | D243M/D272Q/T345A/T348K/C363E/A390S/A395L | + |
| 285/286 | P113G/D243M/D272Q/V351R/C363E/A390S/A395L | + |
| 287/288 | P113G/D243R/R257V/D272Q/T345A/A390S/E430L | + |
| 289/290 | P113G/D243S/V267R/D272Q/C363E/A390S/E430G | + |
| 291/292 | P113G/D243M/V267R/T348K/C363E/E430L | + |
| 293/294 | P113G/D243S/R257T/V351R/A390G/E430L | + |
| 295/296 | P113G/D243S/R257T/D272N/V351K/A390S | + |
| 297/298 | P113G/D243R/V267R/A390G/E430G | + |
| 299/300 | D243R/R257F/T348K/A395L/E430G | + |
| 301/302 | D243R/T345A/T348K/V429L/E430L | + |
| 303/304 | D243M/R257F/T345A/T348K/A395L | + |
| 305/306 | D243S/R257T/D272N/V351K/V400L/V429L | + |
| 307/308 | P113G/D243M/V267R | + |
| 309/310 | D243M/V267R/V351R | + |
| 311/312 | P113G/A390S/E430G | + |
| 313/314 | D243S/V267R/V351K/V400L | + |
| 315/316 | V429L/E430G | + |

¹ Levels of increased activity were determined relative to the reference polypeptide of SEQ ID NO: 36 and defined as follows: “+” 1.07 to 1.53-fold increased activity; “++” > 1.53-fold increased activity; “+++” > 1.64-fold increased activity.

### Example 10

Variants with Improvements Over SEQ ID NO: 238

SEQ ID NO: 238 as selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble heat-treated cell lysate was generated as described in Example 6, with a lysis temperature of 57° C. The clarified lysate was used to detect ribonuclease inhibitor activity as described in Example 7.

Each sample was evaluated by its residual RNase inhibition activity after heat treatment relative to SEQ ID NO: 238 (activity fold improvement over positive control, FIOP). In this example, activity FIOP is the ratio of SEQ ID NO:238's RFU to the given sample's RFU, both measured after the first qPCR incubation cycle. Activity FIOPs are shown in Table 10.1.

TABLE 10.1
RNase Inhibitor Activity of Variants Relative to SEQ ID NO: 238

| | | Activity FIOP |
|---|---|---|
| | | after 57° C. |
| SEQ ID | Amino Acid Differences | heat treatment |
| NO: | (Relative to SEQ | (Relative to SEQ |
| (nt/aa) | ID NO: 238) | ID NO: 238) |
| 317/318 | Q323E/S453K | +++ |
| 319/320 | Q323E/T348K/N452R | +++ |
| 321/322 | N452R/S453A | +++ |
| 323/324 | Q323E/V351R/E430L/N452R/S453A | +++ |
| 325/326 | E291P | +++ |
| 327/328 | L145E | ++ |
| 329/330 | G228A | ++ |
| 331/332 | Q323E/V429L | ++ |
| 333/334 | L145G | ++ |
| 335/336 | L145M | ++ |
| 337/338 | E38T | + |
| 339/340 | H150Q | + |
| 341/342 | G228R | + |
| 343/344 | T12K | + |
| 345/346 | L173I | + |
| 347/348 | E38C/Q237R | + |
| 349/350 | V40I | + |
| 351/352 | E38R | + |

¹ Levels of increased activity were determined relative to the reference polypeptide of SEQ ID NO: 2 and defined as follows: “+” 1.04 to 1.50-fold increased activity; “++” > 1.50-fold increased activity; “+++” > 1.65-fold increased activity.

### Example 11

Variants with Improvements Over SEQ ID NO: 320

SEQ ID NO: 320 as selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble heat-treated cell lysate was generated as described in Example 6, with a lysis temperature of 61° C. The clarified lysate was used to detect ribonuclease inhibitor activity as described in Example 7.

Each sample was evaluated by its residual RNase inhibition activity after heat treatment relative to SEQ ID NO: 320 (activity fold improvement over positive control, FIOP). In this example, activity FIOP is the ratio of SEQ ID NO:320's RFU to the given sample's RFU, both measured after the first qPCR incubation cycle. Activity FIOPs are shown in Table 11.1.

TABLE 11.1
RNase Inhibitor Activity of Variants Relative to SEQ ID NO: 320

| | | Activity FIOP |
|---|---|---|
| SEQ ID | | after 61° C. heat |
| NO: | Amino Acid Differences | treatment (Relative |
| (nt/aa) | (Relative to SEQ ID NO: 320) | to SEQ ID NO: 320) |
| 353/354 | M13T/L145M/G228A/E291P/K348T/V351R | +++ |
| 355/356 | S453R | +++ |
| 357/358 | T12K/M13T/S453R | +++ |
| 359/360 | L145V/G228R/E291P/S453R | +++ |
| 361/362 | M13T/L145V/S453R | +++ |
| 363/364 | L145M/G228R/E291P/S453R | ++ |
| 365/366 | L145V/E291P/S453R | ++ |
| 367/368 | E38T/L145V/S453R | ++ |
| 369/370 | T12K/L145M/S453R | ++ |
| 371/372 | S453K | ++ |
| 373/374 | E38T/L145I/G228A/S453R | ++ |
| 375/376 | L15R/T28H/H81T/E124A/A221S/R285Q/E317S | + |
| 377/378 | L15R/T28H/S64V/D203E/A221S/R285Q/E317S | + |
| 379/380 | L15R/S57M/H81T/E124A/Q148N/D203E | + |
| 381/382 | T12K/S453K | + |
| 383/384 | S56A/S57T/S64V/H81T/T91K/E124A/D203E/R285Q/E317S | + |
| 385/386 | T66I/P90G/S142H/K176S/A205P/A319G | + |
| 387/388 | G228R/S453K | + |
| 389/390 | L145I/G228A/S453A | + |

¹ Levels of increased activity were determined relative to the reference polypeptide of SEQ ID NO: 320 and defined as follows: “+” 1.19 to 1.50-fold increased activity; “++” >1.50-fold increased activity; “+++” >1.62-fold increased activity.

### Example 12

Variants with Improvements Over SEQ ID NO: 360

SEQ ID NO: 360 as selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble heat-treated cell lysate was generated as described in Example 6, with a lysis temperature of 63.5° C. or 65° C. The clarified lysate was used to detect ribonuclease inhibitor activity as described in Example 7.

Each sample was evaluated by its residual RNase inhibition activity after heat treatment relative to SEQ ID NO: 360 (activity fold improvement over positive control, FIOP). In this example, activity FIOP is the ratio of SEQ ID NO:360's RFU to the given sample's RFU, both measured after the first qPCR incubation cycle. Activity FIOPs are shown in Tables 12.1 and 12.2.

TABLE 12.1 RNase Inhibitor Activity of
Variants Relative to SEQ ID NO: 360

| | | Activity FIOP after |
|---|---|---|
| SEQ ID | | 63.5° C. heat treatment |
| NO: | Amino Acid Differences | (Relative to SEQ ID NO: |
| (nt/aa) | (Relative to SEQ ID NO: 360) | 360) |
| 391/392 | A319T | +++ |
| 393/394 | S37C | +++ |
| 395/396 | Q60K | +++ |
| 397/398 | S342A | +++ |
| 399/400 | S342M | +++ |
| 401/402 | H81V | ++ |
| 403/404 | H81M | ++ |
| 405/406 | M50S | ++ |
| 407/408 | A395L | ++ |
| 409/410 | S64R | ++ |
| 411/412 | R177T | ++ |
| 413/414 | T107D | ++ |
| 415/416 | M50Q | + |
| 417/418 | S37A | + |
| 419/420 | S373A | + |
| 421/422 | A319G | + |
| 423/424 | A205L | + |
| 425/426 | A205I | + |
| 427/428 | A205V | + |
| 429/430 | Q35R | + |
| 431/432 | A78K | + |
| 433/434 | R228L | + |
| 435/436 | H178D | + |
| 437/438 | R285K | + |

¹ Levels of increased activity were determined relative to the reference polypeptide of SEQ ID NO: 360 and defined as follows: “+” 1.14 to 1.22-fold increased activity; “++” >1.22-fold increased activity; “+++” >1.28-fold increased activity.

TABLE 12.2 RNase Inhibitor Activity of Variants Relative to SEQ ID NO: 360

| | | Activity FIOP after 65° C. |
|---|---|---|
| SEQ ID | Amino Acid Differences | heat treatment (Relative |
| NO: (nt/aa) | (Relative to SEQ ID NO: 360) | to SEQ ID NO: 360) |
| 439/440 | S37A/S142Q/Q148N/D203E/A205P/R285Q/A319G | +++ |
| 441/442 | M50S/H81T/P90G/E124A/P147E/Q148N/D203E/A205P/R285Q | +++ |
| 443/444 | T66I/P90G/Q148N/R285Q/E317S/A319G | +++ |
| 445/446 | T28H/S37A/T91K/S142Q/A205P/R285Q | +++ |
| 447/448 | M50S/T91K/E124A/S142Q/P147E/Q148N/D203E/A205P/E317S/A319G | +++ |
| 449/450 | M50S/T66I/P90G/T91K/E124A/S142Q/R285Q/E317S | +++ |
| 451/452 | P147E/Q148N/D203E/R285Q/E317S/A319G | +++ |
| 453/454 | M50S/T66I/H81T/P90G/A205P/A319G | +++ |
| 455/456 | M50S/T66I/E124A/D203E/A205P/E317S/A319G | +++ |
| 457/458 | T28H/T66I/L84I/P90G/T91K/S142Q/A205P/A319G | +++ |
| 459/460 | S37A/T91K/Q148N/A205P/A319G | +++ |
| 461/462 | T28H/T66I/H81T/E124A/P147E/R285Q/A319G | ++ |
| 463/464 | T28H/M50S/T66I/L84I/P90G/P147E/D203E/A205P/E317S | ++ |
| 465/466 | T28H/M50S/P90G/T91K/E124A/S142Q/A319G | ++ |
| 467/468 | S37A/H81T/P147E/Q148N/D203E/A205P | ++ |
| 469/470 | T28H/T66I/H81T/P90G/T91K/E124A/D203E/R285Q/E317S/A319G | ++ |
| 471/472 | T28H/M50S/T66I/H81T/P90G/T91K/Q138L/P147E/Q148N/A205P/R285Q/A319G | ++ |
| 473/474 | T28H/S37A/M50S/E124A/A319G | ++ |
| 475/476 | H81T/P90G/T91K/E124A/R285Q/E317S | ++ |
| 477/478 | M50S/P90G/S142Q/D203E/R285Q | ++ |
| 479/480 | M50S/P90G/E124A/S142Q/A205P/R285Q/A319G | ++ |
| 481/482 | T28H/T66I/P90G/T91K/P147E/Q148N/A205P/A319G/W326L | ++ |
| 483/484 | T66I/H81T/A319G | ++ |
| 485/486 | T28H/T66I/P90G/D203E/A205P/A319G | ++ |
| 487/488 | M50S/T66I/P90G/T91K/A205P | ++ |
| 489/490 | M50S/T66I/P147E/A205P | ++ |
| 491/492 | S37A/H81T/R285Q | ++ |
| 493/494 | M50S/T66I/P90G/T91K/A319G | ++ |
| 495/496 | M50S/P147E/D203E/R285Q | + |
| 497/498 | S37A/M50S/H81T/Q148N | + |
| 499/500 | T28H/T66I/S142Q/A319G | + |
| 501/502 | L15R/T28H/T66I/H81T/P90G/T91K/P147E/Q148N/A319G | + |
| 503/504 | L15R/T28H/H81T/P90G/T91K/S142Q/R285Q/E317S/A319G | + |
| 505/506 | L15R/T28H/S37A/M50S/T66I/E124A/P147E/Q148N/A205P/R285Q | + |
| 507/508 | L15R/T66I/H81T/P147E/Q148N | + |
| 509/510 | T66I/T91K/E124A/S142Q/P147E/Q148N | + |
| 511/512 | L15R/S37A/T66I/H81T/P147E/Q148N/E317S/A319G | + |
| 513/514 | L15R/M50S/P90G/T91K/E124A/S142Q/P147E/R285Q/E317S/A319G | + |
| 515/516 | L15R/M50S/P90G/T91K/S142Q/P147E/Q148N/D203E/A205P | + |
| 517/518 | L15R/T28H/H81T/S142Q/P147E/Q148N/R285Q/E317S/A319G | + |
| 519/520 | L15R/T28H/M50S/T66I/P90G/T91K/D203E/R285Q/A319G | + |
| 521/522 | L15R/T66I/P90G/T91K/P147E/Q148N/A205P/R285Q | + |
| 523/524 | L15R/T28H/P90G/T91K/S142Q/P147E/Q148N/R285Q/E317S/A319G | + |
| 525/526 | L15R/S37A/T91K/Q148N/R285Q/A319G | + |
| 527/528 | H81T/P147E/Q148N/D203E/A205P | + |
| 529/530 | P147E/Q148N/R285Q/A319G | + |
| 531/532 | L15R/M50S/P90G/P147E/Q148N | + |
| 533/534 | L15R/T66I/H81T/P90G/R285Q/A319G | + |
| 535/536 | M50S/H81T/P147E/Q148N/R285Q | + |
| 537/538 | S37A/P147E/Q148N/R285Q | + |
| 539/540 | L15R/S37A/T66I/L84V/P90G/T91K/D203E/R285Q/E317S | + |
| 541/542 | L15R/L84I/P90G/T91K/D203E/R285Q | + |
| 543/544 | S37A/P90G/T91K/P147E | + |
| 545/546 | P90G/T91K/A205P/E317S/A319G | + |
| 547/548 | M50S/Q148N/D203E/E317S | + |
| 549/550 | L15R/T28H/M50S/T66I/H81T/L84I/P90G/R285Q/E317S | + |
| 551/552 | L15R/H81T/P147E | + |
| 553/554 | L15R/R285Q | + |

¹ Levels of increased activity were determined relative to the reference polypeptide of SEQ ID NO: 360 and defined as follows: “+” 1.12 to 1.50-fold increased activity; “++” >1.50-fold increased activity; “+++” >1.67-fold increased activity.

### Example 13

Variants with Improvements Over SEQ ID NO: 442

SEQ ID NO: 442 as selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble heat-treated cell lysate was generated as described in Example 6, with a lysis temperature of 66.7° C. or 67° C. The clarified lysate was used to detect ribonuclease inhibitor activity as described in Example 7.

Each sample was evaluated by its residual RNase inhibition activity after heat treatment relative to SEQ ID NO: 442 (activity fold improvement over positive control, FIOP). In this example, activity FIOP is the ratio of SEQ ID NO:442's RFU to the given sample's RFU, both measured after the indicated qPCR incubation cycle. Activity FIOPs are shown in Tables 13.1 and 13.2.

TABLE 13.1
RNase Inhibitor Activity of Variants Relative to SEQ ID NO: 442

| | | Activity FIOP after |
|---|---|---|
| SEQ ID | | 66.7° C. heat treatment, |
| NO: | Amino Acid Differences | second qPCR cycle (Relative |
| (nt/aa) | (Relative to SEQ ID NO: 442) | to SEQ ID NO: 442) |
| 667/668 | A78T/H260Y | +++ |
| 669/670 | S396A | +++ |
| 671/672 | T345S | +++ |
| 673/674 | S37C | +++ |
| 675/676 | S342H | +++ |
| 677/678 | A395V | +++ |
| 679/680 | Q367V | ++ |
| 681/682 | A395L | ++ |
| 683/684 | Q367L | ++ |
| 685/686 | E38A | ++ |
| 687/688 | T107V | ++ |
| 689/690 | R228K | ++ |
| 691/692 | H260L | ++ |
| 693/694 | S57V | ++ |
| 695/696 | S50M/R51H | ++ |
| 697/698 | Q338V | + |
| 699/700 | Q285R/P291E/G385A | + |
| 701/702 | A175F | + |
| 703/704 | Q60K | + |
| 705/706 | R228Q | + |
| 707/708 | V145S/E147P/N148Q | + |
| 709/710 | A395M | + |
| 711/712 | Q60S | + |
| 713/714 | R177V | + |
| 715/716 | Q367A | + |
| 717/718 | H260S | + |
| 719/720 | T28A | + |
| 721/722 | S118R | + |
| 723/724 | S64V | + |

¹ Levels of increased activity were determined relative to the reference polypeptide of SEQ ID NO: 442 and defined as follows: “+” 1.15 to 1.35-fold increased activity; “++” >1.35-fold increased activity; “+++” >1.50-fold increased activity.

TABLE 13.2
RNase Inhibitor Activity of Variants Relative to SEQ ID NO: 442

| | | Activity FIOP after 67° C. |
|---|---|---|
| SEQ ID | | heat treatment, third |
| NO: | Amino Acid Differences | qPCR cycle (Relative to |
| (nt/aa) | (Relative to SEQ ID NO: 442) | SEQ ID NO: 442) |
| 555/556 | S37A/Q60K/S142Q/K176S/R228L/P291S/E317S | +++ |
| 557/558 | Q60K/S64R/E317S/A319G/S342A | +++ |
| 559/560 | T66I/R228L/A319G/S342M/A395L | +++ |
| 561/562 | S37A/S142Q/P291S/E317S | +++ |
| 563/564 | S64R/T107D/S142Q/P291S/A319G/S342A/A395L | +++ |
| 565/566 | S37A/Q60K/E317S/A319G/S342A/A395L | +++ |
| 567/568 | S37A/Q60K/T107D/S142Q/K176S/E317S/A319G/S342M | +++ |
| 569/570 | S142Q/E317S/A319G/S342M/A395L | +++ |
| 571/572 | K176S/R228L/A319G/A395L | +++ |
| 573/574 | R228L/P291S/S342M/A395L | +++ |
| 575/576 | S64R/A395L | ++ |
| 577/578 | S64R/T107D/S142Q/K176S/R228L/A319G/S342M/A395L | ++ |
| 579/580 | A395E | ++ |
| 581/582 | Q60K/S64R/T107D/S142Q/P291S/A319G/S342M | ++ |
| 583/584 | S37A/T66I/T107D/P291S/E317S | ++ |
| 585/586 | S37A/T66I/T107D/E317S/A395L | ++ |
| 587/588 | S37A/R228L/E317S/A395L | ++ |
| 589/590 | S342A/A395L | ++ |
| 591/592 | T66I/S142Q/A319G/S342M/A395L | ++ |
| 593/594 | T66I/S142Q/R228L/A395L | ++ |
| 595/596 | C363M | ++ |
| 597/598 | S142Q/A395L | ++ |
| 599/600 | Q60K/S64R/R177T/P291S/A395L | ++ |
| 601/602 | T66I/A319G | ++ |
| 603/604 | S64R/T107D/R177T/R228L/P291S/E317S/A395L | ++ |
| 605/606 | S342M/A395L | + |
| 607/608 | S64R/S142Q/R177T/R228L/E317S | + |
| 609/610 | S37C | + |
| 611/612 | H260L | + |
| 613/614 | Q60K/R228L/E317S/A395L | + |
| 615/616 | Q60K/S64R/K176S/E317S/S342M | + |
| 617/618 | R177T/R228L/S342M/A395L | + |
| 619/620 | S64R/S142Q/R177T/E317S/A319G | + |
| 621/622 | Q60K/S64R/T107D/S342M | + |
| 623/624 | Q60K/T107D/S142Q/P291S/E317S/S342M/A395L | + |
| 625/626 | T66I/R228L/A319G | + |
| 627/628 | S64R/R228L/E317S/A319G | + |
| 629/630 | S168F | + |
| 631/632 | A319G/A395L | + |
| 633/634 | S37A/T107D/R228L | + |
| 635/636 | T66I/S142Q | + |
| 637/638 | S37A/S342M | + |
| 639/640 | Q60K/S64R/K176S/R228L/A395L | + |
| 641/642 | Q60K/S64R/S342M | + |
| 643/644 | Q35R | + |
| 645/646 | Q60K/T66I/A395L | + |
| 647/648 | A319G | + |
| 649/650 | P291S/E317S/A319G/S342M/A395L | + |
| 651/652 | S37A/R177T | + |
| 653/654 | T107V | + |
| 655/656 | S37A | + |
| 657/658 | S37A/Q60K/S64R/S142Q/P291S | + |
| 659/660 | S37A/Q60K/S142Q/R228L/P291S | + |
| 661/662 | Q60K/A319G/S342A | + |
| 663/664 | T66I/K176S/E317S/A319G/A395L | + |
| 665/666 | S37A/R177T/R228L | + |

¹ Levels of increased activity were determined relative to the reference polypeptide of SEQ ID NO: 442 and defined as follows: “+” 1.08 to 1.45-fold increased activity; “++” >1.45-fold increased activity; “+++” >1.60-fold increased activity.

### Example 14

Variants with Improvements Over SEQ ID NO: 578

SEQ ID NO: 578 as selected as the parent protein for this round of directed evolution. Libraries of engineered genes were produced using established techniques (e.g., saturation mutagenesis, recombination of previously identified beneficial mutations). The polypeptides encoded by each gene were produced in HTP as described in Example 4, and the soluble heat-treated cell lysate was generated as described in Example 6, with a lysis temperature of 69° C. or 71° C. The clarified lysate was used to detect ribonuclease inhibitor activity as described in Example 7.

Each sample was evaluated by its residual RNase inhibition activity after heat treatment relative to SEQ ID NO: 578 (activity fold improvement over positive control, FIOP). In this example, activity FIOP is the ratio of SEQ ID NO:578's RFU to the given sample's RFU, both measured after the third qPCR incubation cycle. Activity FIOPs are shown in Tables 14.1 and 14.2.

TABLE 14.1
RNase Inhibitor Activity of Variants Relative to SEQ ID NO: 578

| | | Activity FIOP |
|---|---|---|
| | | after 69° C. |
| SEQ ID | | heat treatment |
| NO: | Amino Acid Differences | (Relative to |
| (nt/aa) | (Relative to SEQ ID NO: 578) | SEQ ID NO: 578) |
| 725/726 | T28A/S118R/A175F/H260S/Q338V/Q367L | +++ |
| 727/728 | S118R/S168F/H260Y/S396A | +++ |
| 729/730 | E38A/Q60S/S168F/H260L/Q338V/Q367L | +++ |
| 731/732 | T28A/E38A/S57V/Q60S/H260L/C363M/Q367L | +++ |
| 733/734 | S57V/S118R/H260L/Q338V/Q367L | +++ |
| 735/736 | S57V/S118R/S168F/R177V/H260L/Q338V/Q367L | +++ |
| 737/738 | H260Y/S396A | +++ |
| 739/740 | T28A/S118R/S168F/H260L/Q367L/S396A | ++ |
| 741/742 | T28A/H260L/Q338V | ++ |
| 743/744 | T28A/Q60K/S168I/H260L | ++ |
| 745/746 | S57V/Q60K/S118R/A175F/R177V/H260L/Q338V | ++ |
| 747/748 | T28A/S57V/H260Y/Q338V/Q367L | ++ |
| 749/750 | Q35R/H260S/Q338V/Q367L | ++ |
| 751/752 | S118R/A175F/H260L/Q338V | ++ |
| 753/754 | S168F/H260L | ++ |
| 755/756 | S57V/Q60S/S168F/R177V/H260S/Q338V/Q367L | ++ |
| 757/758 | T28A/S168F/H260S/Q338V | ++ |
| 759/760 | H260Y/Q367L | + |
| 761/762 | T28A/S57V/L137V/H260L | + |
| 763/764 | E38A/Q60K/A175F/H260L/Q338V | + |
| 765/766 | E38A/Q60K/H260S/Q367A/S396A | + |
| 767/768 | S57V/Q60K/H260S/C363M/Q367V | + |
| 769/770 | Q60K/H260L/Q367A | + |
| 771/772 | T28A/E38A/Q60K/H260S/Q367L | + |
| 773/774 | Q60S/S168F | + |
| 775/776 | H260L/C363M/Q367A | + |
| 777/778 | R177V/H260Y/C363M/Q367V | + |
| 779/780 | E38A/H260L | + |
| 781/782 | T28A/Q60K/S118R/R177V/H260Y | + |
| 783/784 | T28A/Q35R/Q60S/S118R/A175F/H260Y | + |
| 785/786 | Q60S/Q367L | + |
| 787/788 | T28A/Q60S/S118R | + |
| 789/790 | S57V/S118R | + |

¹ Levels of increased activity were determined relative to the reference polypeptide of SEQ ID NO: 578 and defined as follows: “+” 1.14 to 1.65-fold increased activity; “++” >1.65-fold increased activity; “+++” >1.78-fold increased activity.

TABLE 14.2
RNase Inhibitor Activity of Variants Relative to SEQ ID NO: 578

| SEQ ID | | Activity FIOP after 71° C. |
|---|---|---|
| NO: | Amino Acid Differences | heat treatment (Relative |
| (nt/aa) | (Relative to SEQ ID NO: 578) | to SEQ ID NO: 578) |
| 791/792 | S118R/H260L/Q338V/S396A | ++ |
| 739/740 | T28A/S118R/S168F/H260L/Q367L/S396A | ++ |
| 793/794 | T28A/E38A/S57V/Q60K/S118R/R177V/H260Y/Q338V/ | ++ |
| | C363M/Q367L/S396A | ++ |
| 795/796 | T28A/E38A/Q60S/H260L/C363M/Q367L | ++ |
| 797/798 | T28A/Q35R/E38A/S57V/S118R/H260L/C363M/Q367L | ++ |
| 799/800 | T28A/S57V/Q60S/S168F/H260L/Q367A | ++ |
| 801/802 | S168F/A175F/H260L/Q338V/Q367L | ++ |
| 803/804 | S57V/Q60S/S168F/H260Y/Q338V/C363M/Q367V/S396A | ++ |
| 805/806 | Q60S/S168F/C363M/Q367L/S396A | ++ |
| 807/808 | T28A/E38A/S57V/Q60K/S118R/S168F/H260L/Q367L | ++ |
| 809/810 | T28A/A175F/H260L/Q367L | + |
| 731/732 | T28A/E38A/S57V/Q60S/H260L/C363M/Q367L | + |
| 811/812 | Q60S/C363M/Q367L/S396A | + |
| 813/814 | H260S/Q338V/Q367L | + |
| 815/816 | T28A/S57V/H260Y/Q338V/C363M/Q367L | + |
| 817/818 | S168F/H260L/Q367L | + |
| 819/820 | Q367L/S396A | + |
| 821/822 | T28A/Q35R/A175F/H260Y/Q367L/S396A | + |
| 823/824 | S57V/Q60K/S168F/H260L/Q367L | + |

¹ Levels of increased activity were determined relative to the reference polypeptide of SEQ ID NO: 578 and defined as follows: “+” 1.19 to 1.54-fold increased activity; “++” >1.54-fold increased activity

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

APPENDIX

| SEQ ID NO: | Amino Acid Differences |
|---|---|
| (nt/aa) | (Relative to SEQ ID NO: 2) |
| 25/26 | R257F/G459E |
| 27/28 | T345A |
| 29/30 | R257F |
| 31/32 | M296L |
| 33/34 | P113G |
| 35/36 | R95E/M296L/G459E |
| 37/38 | R257V |
| 39/40 | R95Q/R257F/M296I |
| 41/42 | S453M |
| 43/44 | T107I/V267K |
| 45/46 | M417L/I422V/G459E |
| 47/48 | A395L |
| 49/50 | A390S |
| 51/52 | R257G/M296I |
| 53/54 | D243C |
| 55/56 | V156M/R257S |
| 57/58 | V351R |
| 59/60 | T348K |
| 61/62 | C363E |
| 63/64 | S453R |
| 65/66 | R95E/R257F/M296I/I422V |
| 67/68 | A395C |
| 69/70 | I94L/T121S/F332L/A390T/V466I |
| 71/72 | R257G/M417L/E419D/G459E |
| 73/74 | N452R |
| 75/76 | R95E/R257F/M296I |
| 77/78 | A377S/A390T |
| 79/80 | S453F |
| 81/82 | N452K |
| 83/84 | D272N |
| 85/86 | D243S |
| 87/88 | Y126V/D386G |
| 89/90 | V267H |
| 91/92 | A390G |
| 93/94 | E430L |
| 95/96 | S453L |
| 97/98 | S453A |
| 99/100 | V230L/V429L |
| 101/102 | A377S/A390S |
| 103/104 | R257T |
| 105/106 | G459K |
| 107/108 | D272Q |
| 109/110 | E430G |
| 111/112 | S453K |
| 113/114 | V83L/T107I/V230L/V267T/V429L |
| 115/116 | V267R |
| 117/118 | T107I/V429L |
| 119/120 | D243R |
| 121/122 | D243M |
| 123/124 | T107A/Q323E/V429L |
| 125/126 | I94L/T121S/I265L/A390T |
| 127/128 | V400L |
| 129/130 | S453W |
| 131/132 | V351K |
| 133/134 | V429L |
| 135/136 | R257F/M296I |
| 137/138 | A395I |
| 139/140 | G459M |
| 141/142 | H81F |
| 143/144 | R95E/M296L/Q323E/A390S/V429L/G459E |
| 145/146 | R95E/R257F/M296L/Q323E/V429L/G459E |
| 147/148 | R95E/R257F/M296L/A390S/G459E |
| 149/150 | R95E/R257F/M296L/Q323E/A390T/G459E |
| 151/152 | R95E/R257F/M296L/Q323E/A390S/V429L/G459E |
| 153/154 | R95E/R257F/M296L/V429L/G459E |
| 155/156 | R95E/R257F/M296L/A377T/A390T/G459E |
| 157/158 | R95E/A135V/R257F/M296L/Q323E/A377S/A390T/G459E |
| 159/160 | R95E/M296L/Q323E/A377T/A390S/V429L/G459E |
| 161/162 | R95E/R257F/M296L/Q323E/A390S/G459E |
| 163/164 | R95E/R257F/M296L/Q323E/A377S/A390S/G459E |
| 165/166 | R95E/R257F/M296L/Q323E/A377T/A390T/G459E |
| 167/168 | R95E/R257F/M296L/A377S/V429L/G459E |
| 169/170 | R95E/R257F/M296L/A390T/G459E |
| 171/172 | R95E/Y158C/R257F/M296L/Q323E/A377T/A390S/G459E |
| 173/174 | R95E/R257F/M296L/A377T/V429L/G459E |
| 175/176 | R95E/R257F/M296L/A377S/A390S/G459E |
| 177/178 | R95E/R257F/M296L/Q323E/A377S/A390T/G459E |
| 179/180 | R95E/R257F/M296L/Q323E/A377T/V429L/G459E |
| 181/182 | R95E/E167K/R257F/M296L/Q323E/A390S/V429L/G459E |
| 183/184 | R95E/M296L/Q323E/A390T/V429L/G459E |
| 185/186 | R95E/R257F/M296L/Q323E/A390T/V429L/G459E |
| 187/188 | R95E/M296L/A377T/A390S/V429L/G459E |
| 189/190 | R95E/R257F/M296L/A377T/A390S/G459E |
| 191/192 | R95E/M296L/A377T/A390S/G459E |
| 193/194 | R95E/R257F/M296L/Q323E/A377S/A390S/V429L/G459E |
| 195/196 | R95E/R257F/M296L/Q323E/A377S/A390T/V429L/G459E |
| 197/198 | R95E/R257F/M296L/Q323E/A377S/V429L/G459E |
| 199/200 | R95E/R257F/M296L/A377S/G459E |
| 201/202 | R95E/M296L/A390T/V429L/G459E |
| 203/204 | R95E/R257F/M296L/Q323E/A377T/A390S/G459E |
| 205/206 | R95E/R257F/M296L/Q323E/A377T/A390S/V429L/G459E |
| 207/208 | R95E/R257F/M296L/A377T/A390T/V429L/G459E |
| 209/210 | R95E/R257F/M296L/A390T/V429L/G459E |
| 211/212 | R95E/R257F/M296L/A377T/A390S/V429L/G459E |
| 213/214 | R95E/R257F/M296L/A377S/A390T/G459E |
| 215/216 | R95E/R257F/M296L/A377S/A390T/V429L/G459E |
| 217/218 | R95E/R257F/M296L/A390S/V429L/G459E |
| 219/220 | R95E/M296L/A377S/A390T/G459E |
| 221/222 | R95E/M296L/A390T/G459E |
| 223/224 | R95E/D243S/M296L/C363E/A390G/G459E |
| 225/226 | R95E/P113G/R257F/M296L/V351K/C363E/V429L/G459E |
| 227/228 | R95E/P113G/R257V/M296L/V351K/A390S/E430G/G459E |
| 229/230 | R95E/P113G/R257V/D272N/M296L/A390S/G459E |
| 231/232 | R95E/P113G/D243M/M296L/V351K/A390S/V429L/G459E |
| 233/234 | R95E/R257V/D272N/M296L/T345A/T348K/G459E |
| 235/236 | R95E/D243S/R257T/M296L/V351R/A390S/G459E |
| 237/238 | R95E/P113G/D243M/R257T/D272N/M296L/A390G/G459E |
| 239/240 | R95E/R257T/D272Q/M296L/C363E/A390G/E430L/G459E |
| 241/242 | R95E/R257V/M296L/V351K/A390G/G459E |
| 243/244 | R95E/P113G/D243S/M296L/A390G/E430G/G459E |
| 245/246 | R95E/P113G/R257V/V267H/M296L/V351R/C363E/V400L/E430L/G459E |
| 247/248 | R95E/P113G/D243M/R257V/M296L/V351R/E430G/G459E |
| 249/250 | R95E/D243M/V267R/M296L/S341C/A390G/A395L/G459E |
| 251/252 | R95E/P113G/D243M/R257T/M296L/T345A/T348K/G459E |
| 253/254 | R95E/P113G/D243S/R257F/M296L/T348K/A390G/G459E |
| 255/256 | R95E/D243S/M296L/T345A/T348K/A390G/G459E |
| 257/258 | R95E/P113G/D243M/R257V/M296L/T345A/E430L/G459E |
| 259/260 | R95E/R257T/M296L/T345A/T348K/V351R/A390S/V429L/E430G/G459E |
| 261/262 | R95E/D272Q/M296L/T345A/A390G/V429L/E430L/G459E |
| 263/264 | R95E/D243M/V267R/M296L/V351K/G459E |
| 265/266 | R95E/D243R/D272N/M296L/T345A/A390G/G459E |
| 267/268 | R95E/P113G/R257T/M296L/C363E/A390G/G459E |
| 269/270 | R95E/D243S/M296L/A390G/V429L/G459E |
| 271/272 | R95E/P113G/D243M/R257F/M296L/V351K/A390G/G459E |
| 273/274 | R95E/P113G/D243M/D272Q/M296L/T345A/T348K/E430G/G459E |
| 275/276 | R95E/D243M/V267R/D272Q/M296L/T348K/A390G/G459E |
| 277/278 | R95E/P113G/R257T/D272Q/M296L/T345A/T348K/C363E/E430L/G459E |
| 279/280 | R95E/P113G/D243R/M296L/A390G/V429L/E430L/G459E |
| 281/282 | R95E/P113G/D243M/R257F/D272N/M296L/V351R/C363E/G459E |
| 283/284 | R95E/D243M/D272Q/M296L/T345A/T348K/C363E/A390S/A395L/G459E |
| 285/286 | R95E/P113G/D243M/D272Q/M296L/V351R/C363E/A390S/A395L/G459E |
| 287/288 | R95E/P113G/D243R/R257V/D272Q/M296L/T345A/A390S/E430L/G459E |
| 289/290 | R95E/P113G/D243S/V267R/D272Q/M296L/C363E/A390S/E430G/G459E |
| 291/292 | R95E/P113G/D243M/V267R/M296L/T348K/C363E/E430L/G459E |
| 293/294 | R95E/P113G/D243S/R257T/M296L/V351R/A390G/E430L/G459E |
| 295/296 | R95E/P113G/D243S/R257T/D272N/M296L/V351K/A390S/G459E |
| 297/298 | R95E/P113G/D243R/V267R/M296L/A390G/E430G/G459E |
| 299/300 | R95E/D243R/R257F/M296L/T348K/A395L/E430G/G459E |
| 301/302 | R95E/D243R/M296L/T345A/T348K/V429L/E430L/G459E |
| 303/304 | R95E/D243M/R257F/M296L/T345A/T348K/A395L/G459E |
| 305/306 | R95E/D243S/R257T/D272N/M296L/V351K/V400L/V429L/G459E |
| 307/308 | R95E/P113G/D243M/V267R/M296L/G459E |
| 309/310 | R95E/D243M/V267R/M296L/V351R/G459E |
| 311/312 | R95E/P113G/M296L/A390S/E430G/G459E |
| 313/314 | R95E/D243S/V267R/M296L/V351K/V400L/G459E |
| 315/316 | R95E/M296L/V429L/E430G/G459E |
| 317/318 | R95E/P113G/D243M/R257T/D272N/M296L/Q323E/A390G/S453K/G459E |
| 319/320 | R95E/P113G/D243M/R257T/D272N/M296L/Q323E/T348K/A390G/N452R/G459E |
| 321/322 | R95E/P113G/D243M/R257T/D272N/M296L/A390G/N452R/S453A/G459E |
| 323/324 | R95E/P113G/D243M/R257T/D272N/M296L/Q323E/V351R/A390G/E430L/N452R/ |
| | S453A/G459E |
| 325/326 | R95E/P113G/D243M/R257T/D272N/E291P/M296L/A390G/G459E |
| 327/328 | R95E/P113G/L145E/D243M/R257T/D272N/M296L/A390G/G459E |
| 329/330 | R95E/P113G/G228A/D243M/R257T/D272N/M296L/A390G/G459E |
| 331/332 | R95E/P113G/D243M/R257T/D272N/M296L/Q323E/A390G/V429L/G459E |
| 333/334 | R95E/P113G/L145G/D243M/R257T/D272N/M296L/A390G/G459E |
| 335/336 | R95E/P113G/L145M/D243M/R257T/D272N/M296L/A390G/G459E |
| 337/338 | E38T/R95E/P113G/D243M/R257T/D272N/M296L/A390G/G459E |
| 339/340 | R95E/P113G/H150Q/D243M/R257T/D272N/M296L/A390G/G459E |
| 341/342 | R95E/P113G/G228R/D243M/R257T/D272N/M296L/A390G/G459E |
| 343/344 | T12K/R95E/P113G/D243M/R257T/D272N/M296L/A390G/G459E |
| 345/346 | R95E/P113G/L173I/D243M/R257T/D272N/M296L/A390G/G459E |
| 347/348 | E38C/R95E/P113G/Q237R/D243M/R257T/D272N/M296L/A390G/G459E |
| 349/350 | V40I/R95E/P113G/D243M/R257T/D272N/M296L/A390G/G459E |
| 351/352 | E38R/R95E/P113G/D243M/R257T/D272N/M296L/A390G/G459E |
| 353/354 | M13T/R95E/P113G/L145M/G228A/D243M/R257T/D272N/E291P/M296L/Q323E/ |
| | V351R/A390G/N452R/G459E |
| 355/356 | R95E/P113G/D243M/R257T/D272N/M296L/Q323E/T348K/A390G/N452R/S453R/ |
| | G459E |
| 357/358 | T12K/M13T/R95E/P113G/D243M/R257T/D272N/M296L/Q323E/T348K/A390G/ |
| | N452R/S453R/G459E |
| 359/360 | R95E/P113G/L145V/G228R/D243M/R257T/D272N/E291P/M296L/Q323E/T348K/ |
| | A390G/N452R/S453R/G459E |
| 361/362 | M13T/R95E/P113G/L145V/D243M/R257T/D272N/M296L/Q323E/T348K/A390G/ |
| | N452R/S453R/G459E |
| 363/364 | R95E/P113G/L145M/G228R/D243M/R257T/D272N/E291P/M296L/Q323E/T348K/ |
| | A390G/N452R/S453R/G459E |
| 365/366 | R95E/P113G/L145V/D243M/R257T/D272N/E291P/M296L/Q323E/T348K/A390G/ |
| | N452R/S453R/G459E |
| 367/368 | E38T/R95E/P113G/L145V/D243M/R257T/D272N/M296L/Q323E/T348K/A390G/ |
| | N452R/S453R/G459E |
| 369/370 | T12K/R95E/P113G/L145M/D243M/R257T/D272N/M296L/Q323E/T348K/A390G/ |
| | N452R/S453R/G459E |
| 371/372 | R95E/P113G/D243M/R257T/D272N/M296L/Q323E/T348K/A390G/N452R/S453K/ |
| | G459E |
| 373/374 | E38T/R95E/P113G/L145I/G228A/D243M/R257T/D272N/M296L/Q323E/T348K/ |
| | A390G/N452R/S453R/G459E |
| 375/376 | L15R/T28H/H81T/R95E/P113G/E124A/A221S/D243M/R257T/D272N/R285Q/M296L/ |
| | E317S/Q323E/T348K/A390G/N452R/G459E |
| 377/378 | L15R/T28H/S64V/R95E/P113G/D203E/A221S/D243M/R257T/D272N/R285Q/M296L/ |
| | E317S/Q323E/T348K/A390G/N452R/G459E |
| 379/380 | L15R/S57M/H81T/R95E/P113G/E124A/Q148N/D203E/D243M/R257T/D272N/M296L/ |
| | Q323E/T348K/A390G/N452R/G459E |
| 381/382 | T12K/R95E/P113G/D243M/R257T/D272N/M296L/Q323E/T348K/A390G/N452R/ |
| | S453K/G459E |
| 383/384 | S56A/S57T/S64V/H81T/T91K/R95E/P113G/E124A/D203E/D243M/R257T/D272N/ |
| | R285Q/M296L/E317S/Q323E/T348K/A390G/N452R/G459E |
| 385/386 | T66I/P90G/R95E/P113G/S142H/K176S/A205P/D243M/R257T/D272N/M296L/A319G/ |
| | Q323E/T348K/A390G/N452R/G459E |
| 387/388 | R95E/P113G/G228R/D243M/R257T/D272N/M296L/Q323E/T348K/A390G/N452R/ |
| | S453K/G459E |
| 389/390 | R95E/P113G/L145I/G228A/D243M/R257T/D272N/M296L/Q323E/T348K/A390G/ |
| | N452R/S453A/G459E |
| 391/392 | R95E/P113G/L145V/G228R/D243M/R257T/D272N/E291P/M296L/A319T/Q323E/ |
| | T348K/A390G/N452R/S453R/G459E |
| 393/394 | S37C/R95E/P113G/L145V/G228R/D243M/R257T/D272N/E291P/M296L/Q323E/ |
| | T348K/A390G/N452R/S453R/G459E |
| 395/396 | Q60K/R95E/P113G/L145V/G228R/D243M/R257T/D272N/E291P/M296L/Q323E/ |
| | T348K/A390G/N452R/S453R/G459E |
| 397/398 | R95E/P113G/L145V/G228R/D243M/R257T/D272N/E291P/M296L/Q323E/S342A/ |
| | T348K/A390G/N452R/S453R/G459E |
| 399/400 | R95E/P113G/L145V/G228R/D243M/R257T/D272N/E291P/M296L/Q323E/S342M/ |
| | T348K/A390G/N452R/S453R/G459E |
| 401/402 | H81V/R95E/P113G/L145V/G228R/D243M/R257T/D272N/E291P/M296L/Q323E/ |
| | T348K/A390G/N452R/S453R/G459E |
| 403/404 | H81M/R95E/P113G/L145V/G228R/D243M/R257T/D272N/E291P/M296L/Q323E/ |
| | T348K/A390G/N452R/S453R/G459E |
| 405/406 | M50S/R95E/P113G/L145V/G228R/D243M/R257T/D272N/E291P/M296L/Q323E/ |
| | T348K/A390G/N452R/S453R/G459E |
| 407/408 | R95E/P113G/L145V/G228R/D243M/R257T/D272N/E291P/M296L/Q323E/T348K/ |
| | A390G/A395L/N452R/S453R/G459E |
| 409/410 | S64R/R95E/P113G/L145V/G228R/D243M/R257T/D272N/E291P/M296L/Q323E/ |
| | T348K/A390G/N452R/S453R/G459E |
| 411/412 | R95E/P113G/L145V/R177T/G228R/D243M/R257T/D272N/E291P/M296L/Q323E/ |
| | T348K/A390G/N452R/S453R/G459E |
| 413/414 | R95E/T107D/P113G/L145V/G228R/D243M/R257T/D272N/E291P/M296L/Q323E/ |
| | T348K/A390G/N452R/S453R/G459E |
| 415/416 | M50Q/R95E/P113G/L145V/G228R/D243M/R257T/D272N/E291P/M296L/Q323E/ |
| | T348K/A390G/N452R/S453R/G459E |
| 417/418 | S37A/R95E/P113G/L145V/G228R/D243M/R257T/D272N/E291P/M296L/Q323E/ |
| | T348K/A390G/N452R/S453R/G459E |
| 419/420 | R95E/P113G/L145V/G228R/D243M/R257T/D272N/E291P/M296L/Q323E/T348K/ |
| | S373A/A390G/N452R/S453R/G459E |
| 421/422 | R95E/P113G/L145V/G228R/D243M/R257T/D272N/E291P/M296L/A319G/Q323E/ |
| | T348K/A390G/N452R/S453R/G459E |
| 423/424 | R95E/P113G/L145V/A205L/G228R/D243M/R257T/D272N/E291P/M296L/Q323E/ |
| | T348K/A390G/N452R/S453R/G459E |
| 425/426 | R95E/P113G/L145V/A205I/G228R/D243M/R257T/D272N/E291P/M296L/Q323E/ |
| | T348K/A390G/N452R/S453R/G459E |
| 427/428 | R95E/P113G/L145V/A205V/G228R/D243M/R257T/D272N/E291P/M296L/Q323E/ |
| | T348K/A390G/N452R/S453R/G459E |
| 429/430 | Q35R/R95E/P113G/L145V/G228R/D243M/R257T/D272N/E291P/M296L/Q323E/ |
| | T348K/A390G/N452R/S453R/G459E |
| 431/432 | A78K/R95E/P113G/L145V/G228R/D243M/R257T/D272N/E291P/M296L/Q323E/ |
| | T348K/A390G/N452R/S453R/G459E |
| 433/434 | R95E/P113G/L145V/G228L/D243M/R257T/D272N/E291P/M296L/Q323E/T348K/ |
| | A390G/N452R/S453R/G459E |
| 435/436 | R95E/P113G/L145V/H178D/G228R/D243M/R257T/D272N/E291P/M296L/Q323E/ |
| | T348K/A390G/N452R/S453R/G459E |
| 437/438 | R95E/P113G/L145V/G228R/D243M/R257T/D272N/R285K/E291P/M296L/Q323E/ |
| | T348K/A390G/N452R/S453R/G459E |
| 439/440 | S37A/R95E/P113G/S142Q/L145V/Q148N/D203E/A205P/G228R/D243M/R257T/ |
| | D272N/R285Q/E291P/M296L/A319G/Q323E/T348K/A390G/N452R/S453R/G459E |
| 441/442 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/N452R/S453R/ |
| | G459E |
| 443/444 | T66I/P90G/R95E/P113G/L145V/Q148N/G228R/D243M/R257T/D272N/R285Q/E291P/ |
| | M296L/E317S/A319G/Q323E/T348K/A390G/N452R/S453R/G459E |
| 445/446 | T28H/S37A/T91K/R95E/P113G/S142Q/L145V/A205P/G228R/D243M/R257T/D272N/ |
| | R285Q/E291P/M296L/Q323E/T348K/A390G/N452R/S453R/G459E |
| 447/448 | M50S/T91K/R95E/P113G/E124A/S142Q/L145V/P147E/Q148N/D203E/A205P/G228R/ |
| | D243M/R257T/D272N/E291P/M296L/E317S/A319G/Q323E/T348K/A390G/N452R/ |
| | S453R/G459E |
| 449/450 | M50S/T66I/P90G/T91K/R95E/P113G/E124A/S142Q/L145V/G228R/D243M/R257T/ |
| | D272N/R285Q/E291P/M296L/E317S/Q323E/T348K/A390G/N452R/S453R/G459E |
| 451/452 | R95E/P113G/L145V/P147E/Q148N/D203E/G228R/D243M/R257T/D272N/R285Q/ |
| | E291P/M296L/E317S/A319G/Q323E/T348K/A390G/N452R/S453R/G459E |
| 453/454 | M50S/T66I/H81T/P90G/R95E/P113G/L145V/A205P/G228R/D243M/R257T/D272N/ |
| | E291P/M296L/A319G/Q323E/T348K/A390G/N452R/S453R/G459E |
| 455/456 | M50S/T66I/R95E/P113G/E124A/L145V/D203E/A205P/G228R/D243M/R257T/D272N/ |
| | E291P/M296L/E317S/A319G/Q323E/T348K/A390G/N452R/S453R/G459E |
| 457/458 | T28H/T66I/L84I/P90G/T91K/R95E/P113G/S142Q/L145V/A205P/G228R/D243M/ |
| | R257T/D272N/E291P/M296L/A319G/Q323E/T348K/A390G/N452R/S453R/G459E |
| 459/460 | S37A/T91K/R95E/P113G/L145V/Q148N/A205P/G228R/D243M/R257T/D272N/E291P/ |
| | M296L/A319G/Q323E/T348K/A390G/N452R/S453R/G459E |
| 461/462 | T28H/T66I/H81T/R95E/P113G/E124A/L145V/P147E/G228R/D243M/R257T/D272N/ |
| | R285Q/E291P/M296L/A319G/Q323E/T348K/A390G/N452R/S453R/G459E |
| 463/464 | T28H/M50S/T66I/L84I/P90G/R95E/P113G/L145V/P147E/D203E/A205P/G228R/D243M/ |
| | R257T/D272N/E291P/M296L/E317S/Q323E/T348K/A390G/N452R/S453R/G459E |
| 465/466 | T28H/M50S/P90G/T91K/R95E/P113G/E124A/S142Q/L145V/G228R/D243M/R257T/ |
| | D272N/E291P/M296L/A319G/Q323E/T348K/A390G/N452R/S453R/G459E |
| 467/468 | S37A/H81T/R95E/P113G/L145V/P147E/Q148N/D203E/A205P/G228R/D243M/R257T/ |
| | D272N/E291P/M296L/Q323E/T348K/A390G/N452R/S453R/G459E |
| 469/470 | T28H/T66I/H81T/P90G/T91K/R95E/P113G/E124A/L145V/D203E/G228R/D243M/ |
| | R257T/D272N/R285Q/E291P/M296L/E317S/A319G/Q323E/T348K/A390G/N452R/S453R/ |
| | G459E |
| 471/472 | T28H/M50S/T66I/H81T/P90G/T91K/R95E/P113G/Q138L/L145V/P147E/Q148N/A205P/ |
| | G228R/D243M/R257T/D272N/R285Q/E291P/M296L/A319G/Q323E/T348K/A390G/ |
| | N452R/S453R/G459E |
| 473/474 | T28H/S37A/M50S/R95E/P113G/E124A/L145V/G228R/D243M/R257T/D272N/E291P/ |
| | M296L/A319G/Q323E/T348K/A390G/N452R/S453R/G459E |
| 475/476 | H81T/P90G/T91K/R95E/P113G/E124A/L145V/G228R/D243M/R257T/D272N/R285Q/ |
| | E291P/M296L/E317S/Q323E/T348K/A390G/N452R/S453R/G459E |
| 477/478 | M50S/P90G/R95E/P113G/S142Q/L145V/D203E/G228R/D243M/R257T/D272N/R285Q/ |
| | E291P/M296L/Q323E/T348K/A390G/N452R/S453R/G459E |
| 479/480 | M50S/P90G/R95E/P113G/E124A/S142Q/L145V/A205P/G228R/D243M/R257T/D272N/ |
| | R285Q/E291P/M296L/A319G/Q323E/T348K/A390G/N452R/S453R/G459E |
| 481/482 | T28H/T66I/P90G/T91K/R95E/P113G/L145V/P147E/Q148N/A205P/G228R/D243M/ |
| | R257T/D272N/E291P/M296L/A319G/Q323E/W326L/T348K/A390G/N452R/S453R/G459E |
| 483/484 | T66I/H81T/R95E/P113G/L145V/G228R/D243M/R257T/D272N/E291P/M296L/A319G/ |
| | Q323E/T348K/A390G/N452R/S453R/G459E |
| 485/486 | T28H/T66I/P90G/R95E/P113G/L145V/D203E/A205P/G228R/D243M/R257T/D272N/ |
| | E291P/M296L/A319G/Q323E/T348K/A390G/N452R/S453R/G459E |
| 487/488 | M50S/T66I/P90G/T91K/R95E/P113G/L145V/A205P/G228R/D243M/R257T/D272N/ |
| | E291P/M296L/Q323E/T348K/A390G/N452R/S453R/G459E |
| 489/490 | M50S/T66I/R95E/P113G/L145V/P147E/A205P/G228R/D243M/R257T/D272N/E291P/ |
| | M296L/Q323E/T348K/A390G/N452R/S453R/G459E |
| 491/492 | S37A/H81T/R95E/P113G/L145V/G228R/D243M/R257T/D272N/R285Q/E291P/M296L/ |
| | Q323E/T348K/A390G/N452R/S453R/G459E |
| 493/494 | M50S/T66I/P90G/T91K/R95E/P113G/L145V/G228R/D243M/R257T/D272N/E291P/ |
| | M296L/A319G/Q323E/T348K/A390G/N452R/S453R/G459E |
| 495/496 | M50S/R95E/P113G/L145V/P147E/D203E/G228R/D243M/R257T/D272N/R285Q/E291P/ |
| | M296L/Q323E/T348K/A390G/N452R/S453R/G459E |
| 497/498 | S37A/M50S/H81T/R95E/P113G/L145V/Q148N/G228R/D243M/R257T/D272N/E291P/ |
| | M296L/Q323E/T348K/A390G/N452R/S453R/G459E |
| 499/500 | T28H/T66I/R95E/P113G/S142Q/L145V/G228R/D243M/R257T/D272N/E291P/M296L/ |
| | A319G/Q323E/T348K/A390G/N452R/S453R/G459E |
| 501/502 | L15R/T28H/T66I/H81T/P90G/T91K/R95E/P113G/L145V/P147E/Q148N/G228R/D243M/ |
| | R257T/D272N/E291P/M296L/A319G/Q323E/T348K/A390G/N452R/S453R/G459E |
| 503/504 | L15R/T28H/H81T/P90G/T91K/R95E/P113G/S142Q/L145V/G228R/D243M/R257T/ |
| | D272N/R285Q/E291P/M296L/E317S/A319G/Q323E/T348K/A390G/N452R/S453R/G459E |
| 505/506 | L15R/T28H/S37A/M50S/T66I/R95E/P113G/E124A/L145V/P147E/Q148N/A205P/G228R/ |
| | D243M/R257T/D272N/R285Q/E291P/M296L/Q323E/T348K/A390G/N452R/S453R/ |
| | G459E |
| 507/508 | L15R/T66I/H81T/R95E/P113G/L145V/P147E/Q148N/G228R/D243M/R257T/D272N/ |
| | E291P/M296L/Q323E/T348K/A390G/N452R/S453R/G459E |
| 509/510 | T66I/T91K/R95E/P113G/E124A/S142Q/L145V/P147E/Q148N/G228R/D243M/R257T/ |
| | D272N/E291P/M296L/Q323E/T348K/A390G/N452R/S453R/G459E |
| 511/512 | L15R/S37A/T66I/H81T/R95E/P113G/L145V/P147E/Q148N/G228R/D243M/R257T/ |
| | D272N/E291P/M296L/E317S/A319G/Q323E/T348K/A390G/N452R/S453R/G459E |
| 513/514 | L15R/M50S/P90G/T91K/R95E/P113G/E124A/S142Q/L145V/P147E/G228R/D243M/ |
| | R257T/D272N/R285Q/E291P/M296L/E317S/A319G/Q323E/T348K/A390G/N452R/ |
| | S453R/G459E |
| 515/516 | L15R/M50S/P90G/T91K/R95E/P113G/S142Q/L145V/P147E/Q148N/D203E/A205P/ |
| | G228R/D243M/R257T/D272N/E291P/M296L/Q323E/T348K/A390G/N452R/S453R/G459E |
| 517/518 | L15R/T28H/H81T/R95E/P113G/S142Q/L145V/P147E/Q148N/G228R/D243M/R257T/ |
| | D272N/R285Q/E291P/M296L/E317S/A319G/Q323E/T348K/A390G/N452R/S453R/G459E |
| 519/520 | L15R/T28H/M50S/T66I/P90G/T91K/R95E/P113G/L145V/D203E/G228R/D243M/R257T/ |
| | D272N/R285Q/E291P/M296L/A319G/Q323E/T348K/A390G/N452R/S453R/G459E |
| 521/522 | L15R/T66I/P90G/T91K/R95E/P113G/L145V/P147E/Q148N/A205P/G228R/D243M/ |
| | R257T/D272N/R285Q/E291P/M296L/Q323E/T348K/A390G/N452R/S453R/G459E |
| 523/524 | L15R/T28H/P90G/T91K/R95E/P113G/S142Q/L145V/P147E/Q148N/G228R/D243M/ |
| | R257T/D272N/R285Q/E291P/M296L/E317S/A319G/Q323E/T348K/A390G/N452R/ |
| | S453R/G459E |
| 525/526 | L15R/S37A/T91K/R95E/P113G/L145V/Q148N/G228R/D243M/R257T/D272N/R285Q/ |
| | E291P/M296L/A319G/Q323E/T348K/A390G/N452R/S453R/G459E |
| 527/528 | H81T/R95E/P113G/L145V/P147E/Q148N/D203E/A205P/G228R/D243M/R257T/ |
| | D272N/E291P/M296L/Q323E/T348K/A390G/N452R/S453R/G459E |
| 529/530 | R95E/P113G/L145V/P147E/Q148N/G228R/D243M/R257T/D272N/R285Q/E291P/ |
| | M296L/A319G/Q323E/T348K/A390G/N452R/S453R/G459E |
| 531/532 | L15R/M50S/P90G/R95E/P113G/L145V/P147E/Q148N/G228R/D243M/R257T/D272N/ |
| | E291P/M296L/Q323E/T348K/A390G/N452R/S453R/G459E |
| 533/534 | L15R/T66I/H81T/P90G/R95E/P113G/L145V/G228R/D243M/R257T/D272N/R285Q/ |
| | E291P/M296L/A319G/Q323E/T348K/A390G/N452R/S453R/G459E |
| 535/536 | M50S/H81T/R95E/P113G/L145V/P147E/Q148N/G228R/D243M/R257T/D272N/R285Q/ |
| | E291P/M296L/Q323E/T348K/A390G/N452R/S453R/G459E |
| 537/538 | S37A/R95E/P113G/L145V/P147E/Q148N/G228R/D243M/R257T/D272N/R285Q/E291P/ |
| | M296L/Q323E/T348K/A390G/N452R/S453R/G459E |
| 539/540 | L15R/S37A/T66I/L84V/P90G/T91K/R95E/P113G/L145V/D203E/G228R/D243M/R257T/ |
| | D272N/R285Q/E291P/M296L/E317S/Q323E/T348K/A390G/N452R/S453R/G459E |
| 541/542 | L15R/L84I/P90G/T91K/R95E/P113G/L145V/D203E/G228R/D243M/R257T/D272N/ |
| | R285Q/E291P/M296L/Q323E/T348K/A390G/N452R/S453R/G459E |
| 543/544 | S37A/P90G/T91K/R95E/P113G/L145V/P147E/G228R/D243M/R257T/D272N/E291P/ |
| | M296L/Q323E/T348K/A390G/N452R/S453R/G459E |
| 545/546 | P90G/T91K/R95E/P113G/L145V/A205P/G228R/D243M/R257T/D272N/E291P/M296L/ |
| | E317S/A319G/Q323E/T348K/A390G/N452R/S453R/G459E |
| 547/548 | M50S/R95E/P113G/L145V/Q148N/D203E/G228R/D243M/R257T/D272N/E291P/ |
| | M296L/E317S/Q323E/T348K/A390G/N452R/S453R/G459E |
| 549/550 | L15R/T28H/M50S/T66I/H81T/L84I/P90G/R95E/P113G/L145V/G228R/D243M/R257T/ |
| | D272N/R285Q/E291P/M296L/E317S/Q323E/T348K/A390G/N452R/S453R/G459E |
| 551/552 | L15R/H81T/R95E/P113G/L145V/P147E/G228R/D243M/R257T/D272N/E291P/M296L/ |
| | Q323E/T348K/A390G/N452R/S453R/G459E |
| 553/554 | L15R/R95E/P113G/L145V/G228R/D243M/R257T/D272N/R285Q/E291P/M296L/ |
| | Q323E/T348K/A390G/N452R/S453R/G459E |
| 555/556 | S37A/M50S/Q60K/H81T/P90G/R95E/P113G/E124A/S142Q/L145V/P147E/Q148N/K176S/ |
| | D203E/A205P/G228L/D243M/R257T/D272N/R285Q/E291S/M296L/E317S/Q323E/ |
| | T348K/A390G/N452R/S453R/G459E |
| 557/558 | M50S/Q60K/S64R/H81T/P90G/R95E/P113G/E124A/L145V/P147E/Q148N/D203E/A205P/ |
| | G228R/D243M/R257T/D272N/R285Q/E291P/M296L/E317S/A319G/Q323E/S342A/ |
| | T348K/A390G/N452R/S453R/G459E |
| 559/560 | M50S/T66I/H81T/P90G/R95E/P113G/E124A/L145V/P147E/Q148N/D203E/A205P/ |
| | G228L/D243M/R257T/D272N/R285Q/E291P/M296L/A319G/Q323E/S342M/T348K/A390G/ |
| | A395L/N452R/S453R/G459E |
| 561/562 | S37A/M50S/H81T/P90G/R95E/P113G/E124A/S142Q/L145V/P147E/Q148N/D203E/ |
| | A205P/G228R/D243M/R257T/D272N/R285Q/E291S/M296L/E317S/Q323E/T348K/A390G/ |
| | N452R/S453R/G459E |
| 563/564 | M50S/S64R/H81T/P90G/R95E/T107D/P113G/E124A/S142Q/L145V/P147E/Q148N/ |
| | D203E/A205P/G228R/D243M/R257T/D272N/R285Q/E291S/M296L/A319G/Q323E/ |
| | S342A/T348K/A390G/A395L/N452R/S453R/G459E |
| 565/566 | S37A/M50S/Q60K/H81T/P90G/R95E/P113G/E124A/L145V/P147E/Q148N/D203E/A205P/ |
| | G228R/D243M/R257T/D272N/R285Q/E291P/M296L/E317S/A319G/Q323E/S342A/ |
| | T348K/A390G/A395L/N452R/S453R/G459E |
| 567/568 | S37A/M50S/Q60K/H81T/P90G/R95E/T107D/P113G/E124A/S142Q/L145V/P147E/Q148N/ |
| | K176S/D203E/A205P/G228R/D243M/R257T/D272N/R285Q/E291P/M296L/E317S/ |
| | A319G/Q323E/S342M/T348K/A390G/N452R/S453R/G459E |
| 569/570 | M50S/H81T/P90G/R95E/P113G/E124A/S142Q/L145V/P147E/Q148N/D203E/A205P/ |
| | G228R/D243M/R257T/D272N/R285Q/E291P/M296L/E317S/A319G/Q323E/S342M/ |
| | T348K/A390G/A395L/N452R/S453R/G459E |
| 571/572 | M50S/H81T/P90G/R95E/P113G/E124A/L145V/P147E/Q148N/K176S/D203E/A205P/ |
| | G228L/D243M/R257T/D272N/R285Q/E291P/M296L/A319G/Q323E/T348K/A390G/ |
| | A395L/N452R/S453R/G459E |
| 573/574 | M50S/H81T/P90G/R95E/P113G/E124A/L145V/P147E/Q148N/D203E/A205P/G228L/ |
| | D243M/R257T/D272N/R285Q/E291S/M296L/Q323E/S342M/T348K/A390G/A395L/ |
| | N452R/S453R/G459E |
| 575/576 | M50S/S64R/H81T/P90G/R95E/P113G/E124A/L145V/P147E/Q148N/D203E/A205P/ |
| | G228R/D243M/R257T/D272N/R285Q/E291P/M296L/Q323E/T348K/A390G/A395L/ |
| | N452R/S453R/G459E |
| 577/578 | M50S/S64R/H81T/P90G/R95E/T107D/P113G/E124A/S142Q/L145V/P147E/Q148N/ |
| | K176S/D203E/A205P/G228L/D243M/R257T/D272N/R285Q/E291P/M296L/A319G/ |
| | Q323E/S342M/T348K/A390G/A395L/N452R/S453R/G459E |
| 579/580 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/A395E/N452R/ |
| | S453R/G459E |
| 581/582 | M50S/Q60K/S64R/H81T/P90G/R95E/T107D/P113G/E124A/S142Q/L145V/P147E/ |
| | Q148N/D203E/A205P/G228R/D243M/R257T/D272N/R285Q/E291S/M296L/A319G/Q323E/ |
| | S342M/T348K/A390G/N452R/S453R/G459E |
| 583/584 | S37A/M50S/T66I/H81T/P90G/R95E/T107D/P113G/E124A/L145V/P147E/Q148N/ |
| | D203E/A205P/G228R/D243M/R257T/D272N/R285Q/E291S/M296L/E317S/Q323E/T348K/ |
| | A390G/N452R/S453R/G459E |
| 585/586 | S37A/M50S/T66I/H81T/P90G/R95E/T107D/P113G/E124A/L145V/P147E/Q148N/D203E/ |
| | A205P/G228R/D243M/R257T/D272N/R285Q/E291P/M296L/E317S/Q323E/T348K/ |
| | A390G/A395L/N452R/S453R/G459E |
| 587/588 | S37A/M50S/H81T/P90G/R95E/P113G/E124A/L145V/P147E/Q148N/D203E/A205P/ |
| | G228L/D243M/R257T/D272N/R285Q/E291P/M296L/E317S/Q323E/T348K/A390G/A395L/ |
| | N452R/S453R/G459E |
| 589/590 | M50S/H81T/P90G/R95E/P113G/E124A/L145V/P147E/Q148N/D203E/A205P/G228R/ |
| | D243M/R257T/D272N/R285Q/E291P/M296L/Q323E/S342A/T348K/A390G/A395L/N452R/ |
| | S453R/G459E |
| 591/592 | M50S/T66I/H81T/P90G/R95E/P113G/E124A/S142Q/L145V/P147E/Q148N/D203E/ |
| | A205P/G228R/D243M/R257T/D272N/R285Q/E291P/M296L/A319G/Q323E/S342M/ |
| | T348K/A390G/A395L/N452R/S453R/G459E |
| 593/594 | M50S/T66I/H81T/P90G/R95E/P113G/E124A/S142Q/L145V/P147E/Q148N/D203E/ |
| | A205P/G228L/D243M/R257T/D272N/R285Q/E291P/M296L/Q323E/T348K/A390G/A395L/ |
| | N452R/S453R/G459E |
| 595/596 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/C363M/A390G/N452R/ |
| | S453R/G459E |
| 597/598 | M50S/H81T/P90G/R95E/P113G/E124A/S142Q/L145V/P147E/Q148N/D203E/A205P/ |
| | G228R/D243M/R257T/D272N/R285Q/E291P/M296L/Q323E/T348K/A390G/A395L/ |
| | N452R/S453R/G459E |
| 599/600 | M50S/Q60K/S64R/H81T/P90G/R95E/P113G/E124A/L145V/P147E/Q148N/R177T/D203E/ |
| | A205P/G228R/D243M/R257T/D272N/R285Q/E291S/M296L/Q323E/T348K/A390G/ |
| | A395L/N452R/S453R/G459E |
| 601/602 | M50S/T66I/H81T/P90G/R95E/P113G/E124A/L145V/P147E/Q148N/D203E/A205P/ |
| | G228R/D243M/R257T/D272N/R285Q/E291P/M296L/A319G/Q323E/T348K/A390G/N452R/ |
| | S453R/G459E |
| 603/604 | M50S/S64R/H81T/P90G/R95E/T107D/P113G/E124A/L145V/P147E/Q148N/R177T/ |
| | D203E/A205P/G228L/D243M/R257T/D272N/R285Q/E291S/M296L/E317S/Q323E/T348K/ |
| | A390G/A395L/N452R/S453R/G459E |
| 605/606 | M50S/H81T/P90G/R95E/P113G/E124A/L145V/P147E/Q148N/D203E/A205P/G228R/ |
| | D243M/R257T/D272N/R285Q/E291P/M296L/Q323E/S342M/T348K/A390G/A395L/ |
| | N452R/S453R/G459E |
| 607/608 | M50S/S64R/H81T/P90G/R95E/P113G/E124A/S142Q/L145V/P147E/Q148N/R177T/ |
| | D203E/A205P/G228L/D243M/R257T/D272N/R285Q/E291P/M296L/E317S/Q323E/T348K/ |
| | A390G/N452R/S453R/G459E |
| 609/610 | S37C/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 611/612 | M50S/H81T/P90G/R95E/P113G/E124A/L145V/P147E/Q148N/D203E/A205P/G228R/ |
| | D243M/R257T/H260L/D272N/R285Q/E291P/M296L/Q323E/T348K/A390G/N452R/ |
| | S453R/G459E |
| 613/614 | M50S/Q60K/H81T/P90G/R95E/P113G/E124A/L145V/P147E/Q148N/D203E/A205P/ |
| | G228L/D243M/R257T/D272N/R285Q/E291P/M296L/E317S/Q323E/T348K/A390G/A395L/ |
| | N452R/S453R/G459E |
| 615/616 | M50S/Q60K/S64R/H81T/P90G/R95E/P113G/E124A/L145V/P147E/Q148N/K176S/D203E/ |
| | A205P/G228R/D243M/R257T/D272N/R285Q/E291P/M296L/E317S/Q323E/S342M/ |
| | T348K/A390G/N452R/S453R/G459E |
| 617/618 | M50S/H81T/P90G/R95E/P113G/E124A/L145V/P147E/Q148N/R177T/D203E/A205P/ |
| | G228L/D243M/R257T/D272N/R285Q/E291P/M296L/Q323E/S342M/T348K/A390G/ |
| | A395L/N452R/S453R/G459E |
| 619/620 | M50S/S64R/H81T/P90G/R95E/P113G/E124A/S142Q/L145V/P147E/Q148N/R177T/ |
| | D203E/A205P/G228R/D243M/R257T/D272N/R285Q/E291P/M296L/E317S/A319G/Q323E/ |
| | T348K/A390G/N452R/S453R/G459E |
| 621/622 | M50S/Q60K/S64R/H81T/P90G/R95E/T107D/P113G/E124A/L145V/P147E/Q148N/D203E/ |
| | A205P/G228R/D243M/R257T/D272N/R285Q/E291P/M296L/Q323E/S342M/T348K/ |
| | A390G/N452R/S453R/G459E |
| 623/624 | M50S/Q60K/H81T/P90G/R95E/T107D/P113G/E124A/S142Q/L145V/P147E/Q148N/ |
| | D203E/A205P/G228R/D243M/R257T/D272N/R285Q/E291S/M296L/E317S/Q323E/ |
| | S342M/T348K/A390G/A395L/N452R/S453R/G459E |
| 625/626 | M50S/T66I/H81T/P90G/R95E/P113G/E124A/L145V/P147E/Q148N/D203E/A205P/ |
| | G228L/D243M/R257T/D272N/R285Q/E291P/M296L/A319G/Q323E/T348K/A390G/ |
| | N452R/S453R/G459E |
| 627/628 | M50S/S64R/H81T/P90G/R95E/P113G/E124A/L145V/P147E/Q148N/D203E/A205P/ |
| | G228L/D243M/R257T/D272N/R285Q/E291P/M296L/E317S/A319G/Q323E/T348K/ |
| | A390G/N452R/S453R/G459E |
| 629/630 | M50S/H81T/P90G/R95E/P113G/E124A/L145V/P147E/Q148N/S168F/D203E/A205P/ |
| | G228R/D243M/R257T/D272N/R285Q/E291P/M296L/Q323E/T348K/A390G/N452R/ |
| | S453R/G459E |
| 631/632 | M50S/H81T/P90G/R95E/P113G/E124A/L145V/P147E/Q148N/D203E/A205P/G228R/ |
| | D243M/R257T/D272N/R285Q/E291P/M296L/A319G/Q323E/T348K/A390G/A395L/ |
| | N452R/S453R/G459E |
| 633/634 | S37A/M50S/H81T/P90G/R95E/T107D/P113G/E124A/L145V/P147E/Q148N/D203E/ |
| | A205P/G228L/D243M/R257T/D272N/R285Q/E291P/M296L/Q323E/T348K/A390G/ |
| | N452R/S453R/G459E |
| 635/636 | M50S/T66I/H81T/P90G/R95E/P113G/E124A/S142Q/L145V/P147E/Q148N/D203E/ |
| | A205P/G228R/D243M/R257T/D272N/R285Q/E291P/M296L/Q323E/T348K/A390G/N452R/ |
| | S453R/G459E |
| 637/638 | S37A/M50S/H81T/P90G/R95E/P113G/E124A/L145V/P147E/Q148N/D203E/A205P/ |
| | G228R/D243M/R257T/D272N/R285Q/E291P/M296L/Q323E/S342M/T348K/A390G/ |
| | N452R/S453R/G459E |
| 639/640 | M50S/Q60K/S64R/H81T/P90G/R95E/P113G/E124A/L145V/P147E/Q148N/K176S/D203E/ |
| | A205P/G228L/D243M/R257T/D272N/R285Q/E291P/M296L/Q323E/T348K/A390G/ |
| | A395L/N452R/S453R/G459E |
| 641/642 | M50S/Q60K/S64R/H81T/P90G/R95E/P113G/E124A/L145V/P147E/Q148N/D203E/A205P/ |
| | G228R/D243M/R257T/D272N/R285Q/E291P/M296L/Q323E/S342M/T348K/A390G/ |
| | N452R/S453R/G459E |
| 643/644 | Q35R/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 645/646 | M50S/Q60K/T66I/H81T/P90G/R95E/P113G/E124A/L145V/P147E/Q148N/D203E/A205P/ |
| | G228R/D243M/R257T/D272N/R285Q/E291P/M296L/Q323E/T348K/A390G/A395L/ |
| | N452R/S453R/G459E |
| 647/648 | M50S/H81T/P90G/R95E/P113G/E124A/L145V/P147E/Q148N/D203E/A205P/G228R/ |
| | D243M/R257T/D272N/R285Q/E291P/M296L/A319G/Q323E/T348K/A390G/N452R/ |
| | S453R/G459E |
| 649/650 | M50S/H81T/P90G/R95E/P113G/E124A/L145V/P147E/Q148N/D203E/A205P/G228R/ |
| | D243M/R257T/D272N/R285Q/E291S/M296L/E317S/A319G/Q323E/S342M/T348K/ |
| | A390G/A395L/N452R/S453R/G459E |
| 651/652 | S37A/M50S/H81T/P90G/R95E/P113G/E124A/L145V/P147E/Q148N/R177T/D203E/ |
| | A205P/G228R/D243M/R257T/D272N/R285Q/E291P/M296L/Q323E/T348K/A390G/ |
| | N452R/S453R/G459E |
| 653/654 | M50S/H81T/P90G/R95E/T107V/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 655/656 | S37A/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 657/658 | S37A/M50S/Q60K/S64R/H81T/P90G/R95E/P113G/E124A/S142Q/L145V/P147E/Q148N/ |
| | D203E/A205P/G228R/D243M/R257T/D272N/R285Q/E291S/M296L/Q323E/T348K/ |
| | A390G/N452R/S453R/G459E |
| 659/660 | S37A/M50S/Q60K/H81T/P90G/R95E/P113G/E124A/S142Q/L145V/P147E/Q148N/D203E/ |
| | A205P/G228L/D243M/R257T/D272N/R285Q/E291S/M296L/Q323E/T348K/A390G/ |
| | N452R/S453R/G459E |
| 661/662 | M50S/Q60K/H81T/P90G/R95E/P113G/E124A/L145V/P147E/Q148N/D203E/A205P/ |
| | G228R/D243M/R257T/D272N/R285Q/E291P/M296L/A319G/Q323E/S342A/T348K/ |
| | A390G/N452R/S453R/G459E |
| 663/664 | M50S/T66I/H81T/P90G/R95E/P113G/E124A/L145V/P147E/Q148N/K176S/D203E/ |
| | A205P/G228R/D243M/R257T/D272N/R285Q/E291P/M296L/E317S/A319G/Q323E/T348K/ |
| | A390G/A395L/N452R/S453R/G459E |
| 665/666 | S37A/M50S/H81T/P90G/R95E/P113G/E124A/L145V/P147E/Q148N/R177T/D203E/ |
| | A205P/G228L/D243M/R257T/D272N/R285Q/E291P/M296L/Q323E/T348K/A390G/ |
| | N452R/S453R/G459E |
| 667/668 | M50S/A78T/H81T/P90G/R95E/P113G/E124A/L145V/P147E/Q148N/D203E/A205P/ |
| | G228R/D243M/R257T/H260Y/D272N/R285Q/E291P/M296L/Q323E/T348K/A390G/ |
| | N452R/S453R/G459E |
| 669/670 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/S396A/N452R/ |
| | S453R/G459E |
| 671/672 | M50S/H81T/P90G/R95E/P113G/E124A/L145V/P147E/Q148N/D203E/A205P/G228R/ |
| | D243M/R257T/D272N/R285Q/E291P/M296L/Q323E/T345S/T348K/A390G/N452R/ |
| | S453R/G459E |
| 673/674 | S37C/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 675/676 | M50S/H81T/P90G/R95E/P113G/E124A/L145V/P147E/Q148N/D203E/A205P/G228R/ |
| | D243M/R257T/D272N/R285Q/E291P/M296L/Q323E/S342H/T348K/A390G/N452R/ |
| | S453R/G459E |
| 677/678 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/A395V/N452R/ |
| | S453R/G459E |
| 679/680 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/Q367V/A390G/N452R/ |
| | S453R/G459E |
| 681/682 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/A395L/N452R/ |
| | S453R/G459E |
| 683/684 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/Q367L/A390G/N452R/ |
| | S453R/G459E |
| 685/686 | E38A/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 687/688 | M50S/H81T/P90G/R95E/T107V/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 689/690 | M50S/H81T/P90G/R95E/P113G/E124A/L145V/P147E/Q148N/D203E/A205P/G228K/ |
| | D243M/R257T/D272N/R285Q/E291P/M296L/Q323E/T348K/A390G/N452R/S453R/G459E |
| 691/692 | M50S/H81T/P90G/R95E/P113G/E124A/L145V/P147E/Q148N/D203E/A205P/G228R/ |
| | D243M/R257T/H260L/D272N/R285Q/E291P/M296L/Q323E/T348K/A390G/N452R/ |
| | S453R/G459E |
| 693/694 | M50S/S57V/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 695/696 | R51H/H81T/P90G/R95E/P113G/E124A/L145V/P147E/Q148N/D203E/A205P/G228R/ |
| | D243M/R257T/D272N/R285Q/E291P/M296L/Q323E/T348K/A390G/N452R/S453R/G459E |
| 697/698 | M50S/H81T/P90G/R95E/P113G/E124A/L145V/P147E/Q148N/D203E/A205P/G228R/ |
| | D243M/R257T/D272N/R285Q/E291P/M296L/Q323E/Q338V/T348K/A390G/N452R/ |
| | S453R/G459E |
| 699/700 | M50S/H81T/P90G/R95E/P113G/E124A/L145V/P147E/Q148N/D203E/A205P/G228R/ |
| | D243M/R257T/D272N/M296L/Q323E/T348K/G385A/A390G/N452R/S453R/G459E |
| 701/702 | M50S/H81T/P90G/R95E/P113G/E124A/L145V/P147E/Q148N/A175F/D203E/A205P/ |
| | G228R/D243M/R257T/D272N/R285Q/E291P/M296L/Q323E/T348K/A390G/N452R/ |
| | S453R/G459E |
| 703/704 | M50S/Q60K/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 705/706 | M50S/H81T/P90G/R95E/P113G/E124A/L145V/P147E/Q148N/D203E/A205P/G228Q/ |
| | D243M/R257T/D272N/R285Q/E291P/M296L/Q323E/T348K/A390G/N452R/S453R/G459E |
| 707/708 | M50S/H81T/P90G/R95E/P113G/E124A/L145S/D203E/A205P/G228R/D243M/R257T/ |
| | D272N/R285Q/E291P/M296L/Q323E/T348K/A390G/N452R/S453R/G459E |
| 709/710 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/A390G/A395M/N452R/ |
| | S453R/G459E |
| 711/712 | M50S/Q60S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 713/714 | M50S/H81T/P90G/R95E/P113G/E124A/L145V/P147E/Q148N/R177V/D203E/A205P/ |
| | G228R/D243M/R257T/D272N/R285Q/E291P/M296L/Q323E/T348K/A390G/N452R/ |
| | S453R/G459E |
| 715/716 | M50S/H81T/P90G/R95E/P113G/E124A/L145V/P147E/Q148N/D203E/A205P/G228R/ |
| | D243M/R257T/D272N/R285Q/E291P/M296L/Q323E/T348K/Q367A/A390G/N452R/ |
| | S453R/G459E |
| 717/718 | M50S/H81T/P90G/R95E/P113G/E124A/L145V/P147E/Q148N/D203E/A205P/G228R/ |
| | D243M/R257T/H260S/D272N/R285Q/E291P/M296L/Q323E/T348K/A390G/N452R/ |
| | S453R/G459E |
| 719/720 | T28A/M50S/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 721/722 | M50S/H81T/P90G/R95E/P113G/S118R/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 723/724 | M50S/S64V/H81T/P90G/R95E/P113G/E124A/L145V/P147E/Q148N/D203E/A205P/ |
| | G228R/D243M/R257T/D272N/R285Q/E291P/M296L/Q323E/T348K/A390G/N452R/ |
| | S453R/G459E |
| 725/726 | T28A/M50S/S64R/H81T/P90G/R95E/T107D/P113G/S118R/E124A/S142Q/L145V/ |
| | P147E/Q148N/A175F/K176S/D203E/A205P/G228L/D243M/R257T/H260S/D272N/R285Q/ |
| | E291P/M296L/A319G/Q323E/Q338V/S342M/T348K/Q367L/A390G/A395L/N452R/ |
| | S453R/G459E |
| 727/728 | M50S/S64R/H81T/P90G/R95E/T107D/P113G/S118R/E124A/S142Q/L145V/P147E/ |
| | Q148N/S168F/K176S/D203E/A205P/G228L/D243M/R257T/H260Y/D272N/R285Q/E291P/ |
| | M296L/A319G/Q323E/S342M/T348K/A390G/A395L/S396A/N452R/S453R/G459E |
| 729/730 | E38A/M50S/Q60S/S64R/H81T/P90G/R95E/T107D/P113G/E124A/S142Q/L145V/P147E/ |
| | Q148N/S168F/K176S/D203E/A205P/G228L/D243M/R257T/H260L/D272N/R285Q/ |
| | E291P/M296L/A319G/Q323E/Q338V/S342M/T348K/Q367L/A390G/A395L/N452R/ |
| | S453R/G459E |
| 731/732 | T28A/E38A/M50S/S57V/Q60S/S64R/H81T/P90G/R95E/T107D/P113G/E124A/S142Q/ |
| | L145V/P147E/Q148N/K176S/D203E/A205P/G228L/D243M/R257T/H260L/D272N/R285Q/ |
| | E291P/M296L/A319G/Q323E/S342M/T348K/C363M/Q367L/A390G/A395L/N452R/ |
| | S453R/G459E |
| 733/734 | M50S/S57V/S64R/H81T/P90G/R95E/T107D/P113G/S118R/E124A/S142Q/L145V/P147E/ |
| | Q148N/K176S/D203E/A205P/G228L/D243M/R257T/H260L/D272N/R285Q/E291P/ |
| | M296L/A319G/Q323E/Q338V/S342M/T348K/Q367L/A390G/A395L/N452R/S453R/G459E |
| 735/736 | M50S/S57V/S64R/H81T/P90G/R95E/T107D/P113G/S118R/E124A/S142Q/L145V/P147E/ |
| | Q148N/S168F/K176S/R177V/D203E/A205P/G228L/D243M/R257T/H260L/D272N/ |
| | R285Q/E291P/M296L/A319G/Q323E/Q338V/S342M/T348K/Q367L/A390G/A395L/ |
| | N452R/S453R/G459E |
| 737/738 | M50S/S64R/H81T/P90G/R95E/T107D/P113G/E124A/S142Q/L145V/P147E/Q148N/ |
| | K176S/D203E/A205P/G228L/D243M/R257T/H260Y/D272N/R285Q/E291P/M296L/ |
| | A319G/Q323E/S342M/T348K/A390G/A395L/S396A/N452R/S453R/G459E |
| 739/740 | T28A/M50S/S64R/H81T/P90G/R95E/T107D/P113G/S118R/E124A/S142Q/L145V/ |
| | P147E/Q148N/S168F/K176S/D203E/A205P/G228L/D243M/R257T/H260L/D272N/R285Q/ |
| | E291P/M296L/A319G/Q323E/S342M/T348K/Q367L/A390G/A395L/S396A/N452R/S453R/ |
| | G459E |
| 741/742 | T28A/M50S/S64R/H81T/P90G/R95E/T107D/P113G/E124A/S142Q/L145V/P147E/Q148N/ |
| | K176S/D203E/A205P/G228L/D243M/R257T/H260L/D272N/R285Q/E291P/M296L/ |
| | A319G/Q323E/Q338V/S342M/T348K/A390G/A395L/N452R/S453R/G459E |
| 743/744 | T28A/M50S/Q60K/S64R/H81T/P90G/R95E/T107D/P113G/E124A/S142Q/L145V/P147E/ |
| | Q148N/S168I/K176S/D203E/A205P/G228L/D243M/R257T/H260L/D272N/R285Q/ |
| | E291P/M296L/A319G/Q323E/S342M/T348K/A390G/A395L/N452R/S453R/G459E |
| 745/746 | M50S/S57V/Q60K/S64R/H81T/P90G/R95E/T107D/P113G/S118R/E124A/S142Q/L145V/ |
| | P147E/Q148N/A175F/K176S/R177V/D203E/A205P/G228L/D243M/R257T/H260L/ |
| | D272N/R285Q/E291P/M296L/A319G/Q323E/Q338V/S342M/T348K/A390G/A395L/ |
| | N452R/S453R/G459E |
| 747/748 | T28A/M50S/S57V/S64R/H81T/P90G/R95E/T107D/P113G/E124A/S142Q/L145V/P147E/ |
| | Q148N/K176S/D203E/A205P/G228L/D243M/R257T/H260Y/D272N/R285Q/E291P/ |
| | M296L/A319G/Q323E/Q338V/S342M/T348K/Q367L/A390G/A395L/N452R/S453R/G459E |
| 749/750 | Q35R/M50S/S64R/H81T/P90G/R95E/T107D/P113G/E124A/S142Q/L145V/P147E/Q148N/ |
| | K176S/D203E/A205P/G228L/D243M/R257T/H260S/D272N/R285Q/E291P/M296L/ |
| | A319G/Q323E/Q338V/S342M/T348K/Q367L/A390G/A395L/N452R/S453R/G459E |
| 751/752 | M50S/S64R/H81T/P90G/R95E/T107D/P113G/S118R/E124A/S142Q/L145V/P147E/ |
| | Q148N/A175F/K176S/D203E/A205P/G228L/D243M/R257T/H260L/D272N/R285Q/E291P/ |
| | M296L/A319G/Q323E/Q338V/S342M/T348K/A390G/A395L/N452R/S453R/G459E |
| 753/754 | M50S/S64R/H81T/P90G/R95E/T107D/P113G/E124A/S142Q/L145V/P147E/Q148N/ |
| | S168F/K176S/D203E/A205P/G228L/D243M/R257T/H260L/D272N/R285Q/E291P/M296L/ |
| | A319G/Q323E/S342M/T348K/A390G/A395L/N452R/S453R/G459E |
| 755/756 | M50S/S57V/Q60S/S64R/H81T/P90G/R95E/T107D/P113G/E124A/S142Q/L145V/P147E/ |
| | Q148N/S168F/K176S/R177V/D203E/A205P/G228L/D243M/R257T/H260S/D272N/ |
| | R285Q/E291P/M296L/A319G/Q323E/Q338V/S342M/T348K/Q367L/A390G/A395L/ |
| | N452R/S453R/G459E |
| 757/758 | T28A/M50S/S64R/H81T/P90G/R95E/T107D/P113G/E124A/S142Q/L145V/P147E/Q148N/ |
| | S168F/K176S/D203E/A205P/G228L/D243M/R257T/H260S/D272N/R285Q/E291P/ |
| | M296L/A319G/Q323E/Q338V/S342M/T348K/A390G/A395L/N452R/S453R/G459E |
| 759/760 | M50S/S64R/H81T/P90G/R95E/T107D/P113G/E124A/S142Q/L145V/P147E/Q148N/ |
| | K176S/D203E/A205P/G228L/D243M/R257T/H260Y/D272N/R285Q/E291P/M296L/ |
| | A319G/Q323E/S342M/T348K/Q367L/A390G/A395L/N452R/S453R/G459E |
| 761/762 | T28A/M50S/S57V/S64R/H81T/P90G/R95E/T107D/P113G/E124A/L137V/S142Q/L145V/ |
| | P147E/Q148N/K176S/D203E/A205P/G228L/D243M/R257T/H260L/D272N/R285Q/ |
| | E291P/M296L/A319G/Q323E/S342M/T348K/A390G/A395L/N452R/S453R/G459E |
| 763/764 | E38A/M50S/Q60K/S64R/H81T/P90G/R95E/T107D/P113G/E124A/S142Q/L145V/P147E/ |
| | Q148N/A175F/K176S/D203E/A205P/G228L/D243M/R257T/H260L/D272N/R285Q/ |
| | E291P/M296L/A319G/Q323E/Q338V/S342M/T348K/A390G/A395L/N452R/S453R/G459E |
| 765/766 | E38A/M50S/Q60K/S64R/H81T/P90G/R95E/T107D/P113G/E124A/S142Q/L145V/P147E/ |
| | Q148N/K176S/D203E/A205P/G228L/D243M/R257T/H260S/D272N/R285Q/E291P/ |
| | M296L/A319G/Q323E/S342M/T348K/Q367A/A390G/A395L/S396A/N452R/S453R/G459E |
| 767/768 | M50S/S57V/Q60K/S64R/H81T/P90G/R95E/T107D/P113G/E124A/S142Q/L145V/P147E/ |
| | Q148N/K176S/D203E/A205P/G228L/D243M/R257T/H260S/D272N/R285Q/E291P/ |
| | M296L/A319G/Q323E/S342M/T348K/C363M/Q367V/A390G/A395L/N452R/S453R/G459E |
| 769/770 | M50S/Q60K/S64R/H81T/P90G/R95E/T107D/P113G/E124A/S142Q/L145V/P147E/ |
| | Q148N/K176S/D203E/A205P/G228L/D243M/R257T/H260L/D272N/R285Q/E291P/M296L/ |
| | A319G/Q323E/S342M/T348K/Q367A/A390G/A395L/N452R/S453R/G459E |
| 771/772 | T28A/E38A/M50S/Q60K/S64R/H81T/P90G/R95E/T107D/P113G/E124A/S142Q/L145V/ |
| | P147E/Q148N/K176S/D203E/A205P/G228L/D243M/R257T/H260S/D272N/R285Q/E291P/ |
| | M296L/A319G/Q323E/S342M/T348K/Q367L/A390G/A395L/N452R/S453R/G459E |
| 773/774 | M50S/Q60S/S64R/H81T/P90G/R95E/T107D/P113G/E124A/S142Q/L145V/P147E/Q148N/ |
| | S168F/K176S/D203E/A205P/G228L/D243M/R257T/D272N/R285Q/E291P/M296L/ |
| | A319G/Q323E/S342M/T348K/A390G/A395L/N452R/S453R/G459E |
| 775/776 | M50S/S64R/H81T/P90G/R95E/T107D/P113G/E124A/S142Q/L145V/P147E/Q148N/ |
| | K176S/D203E/A205P/G228L/D243M/R257T/H260L/D272N/R285Q/E291P/M296L/A319G/ |
| | Q323E/S342M/T348K/C363M/Q367A/A390G/A395L/N452R/S453R/G459E |
| 777/778 | M50S/S64R/H81T/P90G/R95E/T107D/P113G/E124A/S142Q/L145V/P147E/Q148N/ |
| | K176S/R177V/D203E/A205P/G228L/D243M/R257T/H260Y/D272N/R285Q/E291P/M296L/ |
| | A319G/Q323E/S342M/T348K/C363M/Q367V/A390G/A395L/N452R/S453R/G459E |
| 779/780 | E38A/M50S/S64R/H81T/P90G/R95E/T107D/P113G/E124A/S142Q/L145V/P147E/Q148N/ |
| | K176S/D203E/A205P/G228L/D243M/R257T/H260L/D272N/R285Q/E291P/M296L/ |
| | A319G/Q323E/S342M/T348K/A390G/A395L/N452R/S453R/G459E |
| 781/782 | T28A/M50S/Q60K/S64R/H81T/P90G/R95E/T107D/P113G/S118R/E124A/S142Q/L145V/ |
| | P147E/Q148N/K176S/R177V/D203E/A205P/G228L/D243M/R257T/H260Y/D272N/ |
| | R285Q/E291P/M296L/A319G/Q323E/S342M/T348K/A390G/A395L/N452R/S453R/G459E |
| 783/784 | T28A/Q35R/M50S/Q60S/S64R/H81T/P90G/R95E/T107D/P113G/S118R/E124A/S142Q/ |
| | L145V/P147E/Q148N/A175F/K176S/D203E/A205P/G228L/D243M/R257T/H260Y/D272N/ |
| | R285Q/E291P/M296L/A319G/Q323E/S342M/T348K/A390G/A395L/N452R/S453R/G459E |
| 785/786 | M50S/Q60S/S64R/H81T/P90G/R95E/T107D/P113G/E124A/S142Q/L145V/P147E/Q148N/ |
| | K176S/D203E/A205P/G228L/D243M/R257T/D272N/R285Q/E291P/M296L/A319G/ |
| | Q323E/S342M/T348K/Q367L/A390G/A395L/N452R/S453R/G459E |
| 787/788 | T28A/M50S/Q60S/S64R/H81T/P90G/R95E/T107D/P113G/S118R/E124A/S142Q/L145V/ |
| | P147E/Q148N/K176S/D203E/A205P/G228L/D243M/R257T/D272N/R285Q/E291P/ |
| | M296L/A319G/Q323E/S342M/T348K/A390G/A395L/N452R/S453R/G459E |
| 789/790 | M50S/S57V/S64R/H81T/P90G/R95E/T107D/P113G/S118R/E124A/S142Q/L145V/P147E/ |
| | Q148N/K176S/D203E/A205P/G228L/D243M/R257T/D272N/R285Q/E291P/M296L/ |
| | A319G/Q323E/S342M/T348K/A390G/A395L/N452R/S453R/G459E |
| 791/792 | M50S/S64R/H81T/P90G/R95E/T107D/P113G/S118R/E124A/S142Q/L145V/P147E/ |
| | Q148N/K176S/D203E/A205P/G228L/D243M/R257T/H260L/D272N/R285Q/E291P/M296L/ |
| | A319G/Q323E/Q338V/S342M/T348K/A390G/A395L/S396A/N452R/S453R/G459E |
| 793/794 | T28A/E38A/M50S/S57V/Q60K/S64R/H81T/P90G/R95E/T107D/P113G/S118R/E124A/ |
| | S142Q/L145V/P147E/Q148N/K176S/R177V/D203E/A205P/G228L/D243M/R257T/H260Y/ |
| | D272N/R285Q/E291P/M296L/A319G/Q323E/Q338V/S342M/T348K/C363M/Q367L/ |
| | A390G/A395L/S396A/N452R/S453R/G459E |
| 795/796 | T28A/E38A/M50S/Q60S/S64R/H81T/P90G/R95E/T107D/P113G/E124A/S142Q/L145V/ |
| | P147E/Q148N/K176S/D203E/A205P/G228L/D243M/R257T/H260L/D272N/R285Q/E291P/ |
| | M296L/A319G/Q323E/S342M/T348K/C363M/Q367L/A390G/A395L/N452R/S453R/G459E |
| 797/798 | T28A/Q35R/E38A/M50S/S57V/S64R/H81T/P90G/R95E/T107D/P113G/S118R/E124A/ |
| | S142Q/L145V/P147E/Q148N/K176S/D203E/A205P/G228L/D243M/R257T/H260L/D272N/ |
| | R285Q/E291P/M296L/A319G/Q323E/S342M/T348K/C363M/Q367L/A390G/A395L/ |
| | N452R/S453R/G459E |
| 799/800 | T28A/M50S/S57V/Q60S/S64R/H81T/P90G/R95E/T107D/P113G/E124A/S142Q/L145V/ |
| | P147E/Q148N/S168F/K176S/D203E/A205P/G228L/D243M/R257T/H260L/D272N/ |
| | R285Q/E291P/M296L/A319G/Q323E/S342M/T348K/Q367A/A390G/A395L/N452R/S453R/ |
| | G459E |
| 801/802 | M50S/S64R/H81T/P90G/R95E/T107D/P113G/E124A/S142Q/L145V/P147E/Q148N/ |
| | S168F/A175F/K176S/D203E/A205P/G228L/D243M/R257T/H260L/D272N/R285Q/E291P/ |
| | M296L/A319G/Q323E/Q338V/S342M/T348K/Q367L/A390G/A395L/N452R/S453R/G459E |
| 803/804 | M50S/S57V/Q60S/S64R/H81T/P90G/R95E/T107D/P113G/E124A/S142Q/L145V/P147E/ |
| | Q148N/S168F/K176S/D203E/A205P/G228L/D243M/R257T/H260Y/D272N/R285Q/E291P/ |
| | M296L/A319G/Q323E/Q338V/S342M/T348K/C363M/Q367V/A390G/A395L/S396A/ |
| | N452R/S453R/G459E |
| 805/806 | M50S/Q60S/S64R/H81T/P90G/R95E/T107D/P113G/E124A/S142Q/L145V/P147E/Q148N/ |
| | S168F/K176S/D203E/A205P/G228L/D243M/R257T/D272N/R285Q/E291P/M296L/A319G/ |
| | Q323E/S342M/T348K/C363M/Q367L/A390G/A395L/S396A/N452R/S453R/G459E |
| 807/808 | T28A/E38A/M50S/S57V/Q60K/S64R/H81T/P90G/R95E/T107D/P113G/S118R/E124A/ |
| | S142Q/L145V/P147E/Q148N/S168F/K176S/D203E/A205P/G228L/D243M/R257T/H260L/ |
| | D272N/R285Q/E291P/M296L/A319G/Q323E/S342M/T348K/Q367L/A390G/A395L/ |
| | N452R/S453R/G459E |
| 809/810 | T28A/M50S/S64R/H81T/P90G/R95E/T107D/P113G/E124A/S142Q/L145V/P147E/Q148N/ |
| | A175F/K176S/D203E/A205P/G228L/D243M/R257T/H260L/D272N/R285Q/E291P/M296L/ |
| | A319G/Q323E/S342M/T348K/Q367L/A390G/A395L/N452R/S453R/G459E |
| 811/812 | M50S/Q60S/S64R/H81T/P90G/R95E/T107D/P113G/E124A/S142Q/L145V/P147E/Q148N/ |
| | K176S/D203E/A205P/G228L/D243M/R257T/D272N/R285Q/E291P/M296L/A319G/ |
| | Q323E/S342M/T348K/C363M/Q367L/A390G/A395L/S396A/N452R/S453R/G459E |
| 813/814 | M50S/S64R/H81T/P90G/R95E/T107D/P113G/E124A/S142Q/L145V/P147E/Q148N/ |
| | K176S/D203E/A205P/G228L/D243M/R257T/H260S/D272N/R285Q/E291P/M296L/A319G/ |
| | Q323E/Q338V/S342M/T348K/Q367L/A390G/A395L/N452R/S453R/G459E |
| 815/816 | T28A/M50S/S57V/S64R/H81T/P90G/R95E/T107D/P113G/E124A/S142Q/L145V/P147E/ |
| | Q148N/K176S/D203E/A205P/G228L/D243M/R257T/H260Y/D272N/R285Q/E291P/ |
| | M296L/A319G/Q323E/Q338V/S342M/T348K/C363M/Q367L/A390G/A395L/N452R/ |
| | S453R/G459E |
| 817/818 | M50S/S64R/H81T/P90G/R95E/T107D/P113G/E124A/S142Q/L145V/P147E/Q148N/ |
| | S168F/K176S/D203E/A205P/G228L/D243M/R257T/H260L/D272N/R285Q/E291P/M296L/ |
| | A319G/Q323E/S342M/T348K/Q367L/A390G/A395L/N452R/S453R/G459E |
| 819/820 | M50S/S64R/H81T/P90G/R95E/T107D/P113G/E124A/S142Q/L145V/P147E/Q148N/ |
| | K176S/D203E/A205P/G228L/D243M/R257T/D272N/R285Q/E291P/M296L/A319G/ |
| | Q323E/S342M/T348K/Q367L/A390G/A395L/S396A/N452R/S453R/G459E |
| 821/822 | T28A/Q35R/M50S/S64R/H81T/P90G/R95E/T107D/P113G/E124A/S142Q/L145V/P147E/ |
| | Q148N/A175F/K176S/D203E/A205P/G228L/D243M/R257T/H260Y/D272N/R285Q/E291P/ |
| | M296L/A319G/Q323E/S342M/T348K/Q367L/A390G/A395L/S396A/N452R/S453R/G459E |
| 823/824 | M50S/S57V/Q60K/S64R/H81T/P90G/R95E/T107D/P113G/E124A/S142Q/L145V/P147E/ |
| | Q148N/S168F/K176S/D203E/A205P/G228L/D243M/R257T/H260L/D272N/R285Q/ |
| | E291P/M296L/A319G/Q323E/S342M/T348K/Q367L/A390G/A395L/N452R/S453R/G459E |

1. An engineered RNase inhibitor, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of an even numbered SEQ ID NO. of SEQ ID NOs: 2 and 26-824, or to a reference sequence corresponding to an even numbered SEQ ID NO. of SEQ ID NOs: 2 and 26-824, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

2. The engineered RNase inhibitor comprising of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 13 to 468 of SEQ ID NO: 2, 36, 238, 320, 360, 442, or 578, or to a reference sequence corresponding to SEQ ID NO: 2, 36, 238, 320, 360, 442, or 578, wherein the amino acid sequence comprises one or more substitutions relative to the reference sequence corresponding to residues 13 to 468 of SEQ ID NO: 2, 36, 238, 320, 360, 442, or 578, or relative to the reference sequence corresponding to SEQ ID NO: 2, 36, 238, 320, 360, 442, or 578.

3-6. (canceled)

7. The engineered RNase inhibitor of claim 1, wherein the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 2, or relative to the reference sequence corresponding to SEQ ID NO: 2.

8. The engineered RNase inhibitor of claim 1, wherein the amino acid sequence of the engineered RNase inhibitor comprises at least a substitution 12K, 13T, 15R, 28A/H, 35R, 37A/C, 38A/C/R/T, 401, 50Q/S, 51H, 56A, 57M/T/V, 60K/S, 64R/V, 66I, 78K/T, 81F/M/T/V, 83L, 84I/V, 90G, 91K, 94L, 95E/Q, 107A/D/I/V, 113G, 118R, 121S, 124A, 126V, 135V, 137V, 138L, 142H/Q, 145E/G/I/M/S/V, 147E, 148N, 150Q, 156M, 158C, 167K, 168F/I, 173I, 175F, 176S, 177T/V, 178D, 203E, 205I/L/P/V, 221S, 228A/L/K/R/Q, 230L, 237R, 243C/M/R/S, 257F/G/S/T/V, 260L/S/Y, 265L, 267H/K/R/T, 272N/Q, 285K/Q, 291E/S, 296I/L, 317S, 319G/T, 323E, 326L, 332L, 338V, 341C, 342A/H/M, 345A/S, 348K, 351K/R, 363E/M, 367A/L/V, 373A, 377S/T, 385A, 386G, 390G/S/T, 395C/E/I/L/M/V, 396A, 400L, 417L, 419D, 422V, 429L, 430G/L, 452K/R, 453A/F/K/L/M/R/W, 459E/K/M, or 466I, or combinations thereof, wherein the amino acid positions are relative to the reference sequence corresponding to residues 13 to 468 of SEQ ID NO: 2, or relative to the reference sequence corresponding to SEQ ID NO: 2.

9-13. (canceled)

14. The engineered RNase inhibitor of claim 1, wherein the amino acid sequence of the engineered RNase inhibitor comprises at least a substitution or substitution set at amino acid position(s) 257/459, 345, 257, 296, 113, 95/296/459, 95/257/296, 453, 107/267, 417/422/459, 395, 390, 257/296, 243, 156/257, 351, 348, 363, 95/257/296/422, 94/121/332/390/466, 257/417/419/459, 452, 377/390, 272, 126/386, 267, 430, 230/429, 459, 83/107/230/267/429, 107/429, 107/323/429, 94/121/265/390, 400, 429, or 81, wherein the amino acid positions are relative to the reference sequence corresponding to residues 13 to 468 of SEQ ID NO: 2, or relative to the reference sequence corresponding to SEQ ID NO: 2.

15-19. (canceled)

20. The engineered RNase inhibitor of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 13 to 468 of an even numbered SEQ ID NO. of SEQ ID NOs: 26-824, or to a reference sequence corresponding to an even numbered SEQ ID NO. of SEQ ID NOs: 26-824, wherein the amino acid sequence comprises one or more substitutions relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

21. The engineered RNase inhibitor of claim 20, wherein the amino acid sequence of the engineered RNase inhibitor comprises at least a substitution at amino acid position 12, 13, 15, 28, 35, 37, 38, 40, 50, 51, 56, 57, 60, 64, 66, 78, 81, 83, 84, 90, 91, 94, 95, 107, 113, 118, 121, 124, 126, 135, 137, 138, 142, 145, 147, 148, 150, 156, 158, 167, 168, 173, 175, 176, 177, 178, 203, 205, 221, 228, 230, 237, 243, 257, 260, 265, 267, 272, 285, 291, 296, 317, 319, 323, 326, 332, 338, 341, 342, 345, 348, 351, 363, 367, 373, 377, 385, 386, 390, 395, 396, 400, 417, 419, 422, 429, 430, 452, 453, 459, or 466, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO: 36, 238, 320, 360, 442, or 578.

22. The engineered RNase inhibitor of claim 21, wherein the amino acid sequence of the engineered RNase inhibitor comprises at least a substitution or an amino acid residue 12K, 13T, 15R, 28A/H, 35R, 37A/C, 38A/C/R/T, 401, 50Q/S/M, 51H, 56A, 57M/T/V, 60K/S, 64R/V, 661, 78K/T, 81F/M/T/V, 83L, 84I/V, 90G, 91K, 94L, 95E/Q, 107A/D/I/V, 113G, 118R, 121S, 124A, 126V, 135V, 137V, 138L, 142H/Q, 145E/G/I/M/S/V, 147E/P, 148Q/N, 150Q, 156M, 158C, 167K, 168F/I, 1731, 175F, 176S, 177T/V, 178D, 203E, 205I/L/P/V, 221S, 228A/L/K/R/Q, 230L, 237R, 243C/M/R/S, 257F/G/S/T/V, 260L/S/Y, 265L, 267H/K/R/T, 272N/Q, 285K/Q, 291E/P/S, 296I/L, 317S, 319G/T, 323E, 326L, 332L, 338V, 341C, 342A/H/M, 345A/S, 348T/K, 351K/R, 363E/M, 367A/L/V, 373A, 377S/T, 385A, 386G, 390G/S/T, 395C/E/I/L/M/V, 396A, 400L, 417L, 419D, 422V, 429L, 430G/L, 452K/R, 453A/F/K/L/M/R/W, 459E/K/M, or 466I, or combinations thereof, wherein the amino acid positions are relative to the reference sequence corresponding to residues 13 to 468 of SEQ ID NO: 36, 238, 320, 360, 442, or 578, or relative to the reference sequence corresponding to SEQ ID NO:
36, 238, 320, 360, 442, or 578.

23-26. (canceled)

27. The engineered RNase inhibitor of claim 20, wherein the amino acid sequence of the engineered RNase inhibitor comprises at least a substitution or substitution set at amino acid positions(s) 323/390/429, 257/323/429, 257/390, 257/323/390, 257/323/390/429, 257/429, 257/377/390, 135/257/323/377/390, 323/377/390/429, 257/323/377/390, 257/377/429, 158/257/323/377/390, 257/323/377/429, 167/257/323/390/429, 377/390/429, 377/390, 257/323/377/390/429, 257/377, 390/429, 257/377/390/429, 257/390/429, 390, 243/363/390, 113/257/351/363/429, 113/257/351/390/430, 113/257/272/390, 113/243/351/390/429, 257/272/345/348, 243/257/351/390, 113/243/257/272/390, 257/272/363/390/430, 257/351/390, 113/243/390/430, 113/257/267/351/363/400/430, 113/243/257/351/430, 243/267/341/390/395, 113/243/257/345/348, 113/243/257/348/390, 243/345/348/390, 113/243/257/345/430, 257/345/348/351/390/429/430, 272/345/390/429/430, 243/267/351, 243/272/345/390, 113/257/363/390, 243/390/429, 113/243/257/351/390, 113/243/272/345/348/430, 243/267/272/348/390, 113/257/272/345/348/363/430, 113/243/390/429/430, 113/243/257/272/351/363, 243/272/345/348/363/390/395, 113/243/272/351/363/390/395, 113/243/257/272/345/390/430, 113/243/267/272/363/390/430, 113/243/267/348/363/430, 113/243/257/351/390/430, 113/243/257/272/351/390, 113/243/267/390/430, 243/257/348/395/430, 243/345/348/429/430, 243/257/345/348/395, 243/257/272/351/400/429, 113/243/267, 113/390/430, 243/267/351/400, or 429/430, wherein the amino acid positions are relative to the reference sequence corresponding to residues 13 to 468 of SEQ ID NO: 36, or relative to the reference sequence corresponding to SEQ ID NO: 36.

28. (canceled)

29. (canceled)

30. The engineered RNase inhibitor of claim 20, wherein the amino acid sequence of the engineered RNase inhibitor comprises at least a substitution or substitution set at amino acid positions(s) 323/453, 323/348/452, 452/453, 323/351/430/452/453, 291, 145, 228, 323/429, 38, 150, 12, 173, 38/237, or 40, wherein the amino acid positions are relative to the reference sequence corresponding to residues 13 to 468 of SEQ ID NO: 238, or relative to the reference sequence corresponding to SEQ ID NO: 238.

31. (canceled)

32. (canceled)

33. The engineered RNase inhibitor of claim 20, wherein the amino acid sequence of the engineered RNase inhibitor comprises at least a substitution or substitution set at amino acid position(s) 13/145/228/291/348/351, 453,12/13/453,145/228/291/453, 13/145/453, 145/291/453, 38/145/453, 12/145/453, 38/145/228/453, 15/28/81/124/221/285/317, 15/28/64/203/221/285/317, 15/57/81/124/148/203, 12/453, 56/57/64/81/91/124/203/285/317, 66/90/142/176/205/319, 228/453, 145/228/45313/145/228/291/348/351, 453, 12/13/453, 145/228/291/453, 13/145/453, 145/291/453, 38/145/453, 12/145/453, 38/145/228/453, 15/28/81/124/221/285/317, 15/28/64/203/221/285/317, 15/57/81/124/148/203, 12/453, 56/57/64/81/91/124/203/285/317, 66/90/142/176/205/319, 228/453, or 145/228/453, wherein the amino acid positions are relative to the reference sequence corresponding to residues 13 to 468 of SEQ ID NO: 320, or relative to the reference sequence corresponding to SEQ ID NO: 320.

34. (canceled)

35. (canceled)

36. The engineered RNase inhibitor of claim 20, wherein the amino acid sequence of the engineered RNase inhibitor comprises at least a substitution or substitution set at amino acid position(s) 319, 37, 60, 342, 81, 50, 395, 64, 177, 107, 373, 205, 35, 78, 228, 178, 285, 37/142/148/203/205/285/319, 50/81/90/124/147/148/203/205/285, 66/90/148/285/317/319, 28/37/91/142/205/285, 50/91/124/142/147/148/203/205/317/319, 50/66/90/91/124/142/285/317, 147/148/203/285/317/319, 50/66/81/90/205/319, 50/66/124/203/205/317/319, 28/66/84/90/91/142/205/319, 37/91/148/205/319, 28/66/81/124/147/285/319, 28/50/66/84/90/147/203/205/317, 28/50/90/91/124/142/319, 37/81/147/148/203/205, 28/66/81/90/91/124/203/285/317/319, 28/50/66/81/90/91/138/147/148/205/285/319, 28/37/50/124/319, 81/90/91/124/285/317, 50/90/142/203/285, 50/90/124/142/205/285/319, 28/66/90/91/147/148/205/319/326, 66/81/319, 28/66/90/203/205/319, 50/66/90/91/205, 50/66/147/205, 37/81/285, 50/66/90/91/319, 50/147/203/285, 37/50/81/148, 28/66/142/319, 15/28/66/81/90/91/147/148/319, 15/28/81/90/91/142/285/317/319, 15/28/37/50/66/124/147/148/205/285, 15/66/81/147/148, 66/91/124/142/147/148, 15/37/66/81/147/148/317/319, 15/50/90/91/124/142/147/285/317/319, 15/50/90/91/142/147/148/203/205, 15/28/81/142/147/148/285/317/319, 15/28/50/66/90/91/203/285/319, 15/66/90/91/147/148/205/285, 15/28/90/91/142/147/148/285/317/319, 15/37/91/148/285/319, 81/147/148/203/205, 147/148/285/319, 15/50/90/147/148, 15/66/81/90/285/319, 50/81/147/148/285, 37/147/148/285, 15/37/66/84/90/91/203/285/317, 15/84/90/91/203/285, 37/90/91/147, 90/91/205/317/319, 50/148/203/317, 15/28/50/66/81/84/90/285/317, 15/81/147, or 15/285, wherein the amino acid positions are relative to the reference sequence corresponding to residues 13 to 468 of SEQ ID NO: 360, or relative to the reference sequence corresponding to SEQ ID NO: 360.

37. (canceled)

38. (canceled)

39. The engineered RNase inhibitor of claim 20, wherein the amino acid sequence of the engineered RNase inhibitor comprises at least a substitution or substitution set at amino acid position(s) 78/260,396, 345, 37, 342, 395, 367, 38, 107, 228, 260, 57, 50/51, 338, 285/291/385, 175, 60, 145/147/148, 177, 28, 118, 64, 37/60/142/176/228/291/317, 60/64/317/319/342, 66/228/319/342/395, 37/142/291/317, 64/107/142/291/319/342/395, 37/60/317/319/342/395, 37/60/107/142/176/317/319/342, 142/317/319/342/395, 176/228/319/395, 228/291/342/395, 64/395, 64/107/142/176/228/319/342/395, 60/64/107/142/291/319/342, 37/66/107/291/317, 37/66/107/317/395, 37/228/317/395, 342/395, 66/142/319/342/395, 66/142/228/395, 363, 142/395, 60/64/177/291/395, 66/319, 64/107/177/228/291/317/395, 64/142/177/228/317, 60/228/317/395, 60/64/176/317/342, 177/228/342/395, 64/142/177/317/319, 60/64/107/342, 60/107/142/291/317/342/395, 66/228/319, 64/228/317/319, 168, 319/395, 37/107/228, 66/142, 37/342, 60/64/176/228/395, 60/64/342, 35, 60/66/395, 319, 291/317/319/342/395, 37/177,107, 37/60/64/142/291, 37/60/142/228/291, 60/319/342, 66/176/317/319/395, or 37/177/228, wherein the amino acid positions are relative to the reference sequence corresponding to residues 13 to 468 of SEQ ID NO: 442, or relative to the reference sequence corresponding to SEQ ID NO: 442.

40. (canceled)

41. (canceled)

42. The engineered RNase inhibitor of claim 20, wherein the amino acid sequence of the engineered RNase inhibitor comprises at least a substitution set at amino acid positions 28/118/175/260/338/367, 118/168/260/396, 38/60/168/260/338/367, 28/38/57/60/260/363/367, 57/118/260/338/367, 57/118/168/177/260/338/367, 260/396, 28/118/168/260/367/396, 28/260/338, 28/60/168/260, 57/60/118/175/177/260/338, 28/57/260/338/367, 35/260/338/367, 118/175/260/338, 168/260, 57/60/168/177/260/338/367, 28/168/260/338, 260/367, 28/57/137/260, 38/60/175/260/338, 38/60/260/367/396, 57/60/260/363/367, 60/260/367, 28/38/60/260/367, 60/168,260/363/367, 177/260/363/367, 38/260, 28/60/118/177/260, 28/35/60/118/175/260, 60/367, 28/60/118, 57/118,118/260/338/396, 28/38/57/60/118/177/260/338/363/367/396, 28/38/60/260/363/367, 28/35/38/57/118/260/363/367, 28/57/60/168/260/367, 168/175/260/338/367, 57/60/168/260/338/363/367/396, 60/168/363/367/396, 28/38/57/60/118/168/260/367, 28/175/260/367, 60/363/367/396, 260/338/367, 28/57/260/338/363/367, 168/260/367, 367/396, 28/35/175/260/367/396, or 57/60/168/260/367, wherein the amino acid positions are relative to the reference sequence corresponding to residues 13 to 468 of SEQ ID NO: 578, or relative to the reference sequence corresponding to SEQ ID NO: 578.

43-46. (canceled)

47. The engineered RNase inhibitor of claim 1, wherein the RNase inhibitor comprises an amino acid sequence comprising residues 13 to 468 of an even numbered SEQ ID NO. of SEQ ID NOs: 26-824, or an amino acid sequence comprising an even numbered SEQ ID NO. of SEQ ID NOs: 26-824.

48. (canceled)

49. The engineered RNase inhibitor of claim 1, wherein the engineered RNase inhibitor has RNase inhibitory activity and has at least one improved property as compared to a reference RNase inhibitor, wherein the improved property is selected from i) increased inhibitory activity against RNase A, ii) increased stability, iii) increased thermostability, iv) increased resistance to oxidation, and v) increased expression as soluble protein, or any combination of i), ii), iii), iv) and v), as compared to a reference RNase inhibitor, and wherein the reference RNase inhibitor has the sequence corresponding to residues 13 to 468 of SEQ ID NO: 2, 36, 238, 320, 360, 442, or 578, or the sequence corresponding to SEQ ID NO: 2, 36, 238, 320, 360, 442, or 578.

50-52. (canceled)

53. An RNase inhibitor comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to
(a) the reference sequence corresponding to residues 12 to 467 of SEQ ID NO: 4; residues 12 to 467 of SEQ ID NO: 6; residues 12 to 472 of SEQ ID NO: 8; residues 12 to 467 of SEQ ID NO: 10;
residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) the reference sequence corresponding to SEQ ID NO: 2, 4, 6, 8, 10, 12, 14, 16, 18, 20, 22, or 24.

54. The RNase inhibitor of claim 53, comprising (a) an amino acid sequence comprising
(a) residues 12 to 467 of SEQ ID NO: 4; residues 12 to 467 of SEQ ID NO: 6; residues 12 to 472 of SEQ ID NO: 8; residues 12 to 467 of SEQ ID NO: 10; residues 12 to 472 of SEQ ID NO: 12; residues 12 to 467 of SEQ ID NO: 14; residues 12 to 467 of SEQ ID NO: 16; residues 12 to 471 of SEQ ID NO: 18; residues 12 to 467 of SEQ ID NO: 20; residues 12 to 467 of SEQ ID NO: 22; or residues 12 to 467 of SEQ ID NO: 24; or
(b) an amino acid sequence comprising SEQ ID NO: 2, 4, 6, 8, 10, 12, 14, 16, 18, 20, 22, or 24.

55. The engineered RNase inhibitor of claim 1, wherein the engineered RNase inhibitor is purified.

56. A recombinant polynucleotide comprising a polynucleotide sequence encoding an engineered RNase inhibitor of claim 1.

57-61. (canceled)

62. An expression vector comprising at least one recombinant polynucleotide of claim 56.

63. (canceled)

64. (canceled)

65. A host cell comprising an expression vector of claim 62.

66. (canceled)

67. (canceled)

68. A method of producing an engineered RNase inhibitor polypeptide in a host cell comprising culturing a host cell of claim 65, under suitable culture conditions such that the encoded engineered RNase inhibitor is produced.

69. (canceled)

70. (canceled)

71. A composition comprising an RNase inhibitor of claim 1.

72-74. (canceled)

75. A method of inhibiting an RNase, comprising contacting an RNase with an engineered RNase inhibitor of claim 1 under conditions suitable for inhibiting the RNase.

76. (canceled)

77. (canceled)

78. A method for preparing a sample containing RNA, comprising contacting a sample with an RNase inhibitor of claim 1.

79. (canceled)

80. (canceled)

81. A kit comprising at least an RNase inhibitor of claim 1.

82. (canceled)
